(19) United States
(12) Patent Application Publication
(10) Pub. No.: US 2022/0108800 A1
(43) Pub. Date: Apr. 7, 2022

(54) TECHNIQUE FOR DETERMINING A STATE OF MULTIPLE SCLEROSIS IN A PATIENT

(71) Applicant: NOVARTIS AG, Basel (CH)

(72) Inventors: Daniela PIANI MEIER, Basel (CH); Davorka Lucia TOMIC, Basel (CH); Chloe TOLLEY, Cheshire (GB); Bryan Martin BENNETT, Middlesex (GB)

(21) Appl. No.: 17/428,221

(22) PCT Filed: Feb. 4, 2020
(86) PCT No.: PCT/EP2020/052745

§ 371 Date: Aug. 3, 2021

Related U.S. Application Data
(60) Provisional application No. 62/801,781, filed on Feb. 6, 2019.

(51) Int. Cl. G16H 50/20; G16H 10/60; G06F 16/903

(52) U.S. Cl. CPC G16H 50/20; G06F 16/90335; G16H 10/60

(57) ABSTRACT

A technique for determining a state of MS (Multiple Sclerosis) in a MS patient, for example Relapsing-Remitting MS (RRMS) or Secondary Progressive MS (SPMS) or an in-transition from RRMS to SPMS state, is presented. In the technique, patient data and age of the patient, and optionally EDSS score and/or T25FW score, is inputted by a user to a processor. The patient data queries comprise items grouped into a first group relating to relapse and recovery, a second group relating to symptoms experienced and a third group relating to impacts experienced by the patient in a predetermined period. Each item has an assigned predetermined weight and comprises a plurality of corresponding predetermined responses. Each response has an assigned predetermined score. Predetermined weights are also assigned for age query and to the EDSS query and/or the T25FW query. The processor, based on the first, second and third group scores and the age score, and the optionally included EDSS score and/or T25FW score generates a total score based on which the state of MS e.g. RRMS or SPMS or in-transition from RRMS to SPMS is determined.

The present invention relates to techniques for determining a state of Multiple Sclerosis (MS) in a patient, and more particularly to techniques for determining progression of MS from relapsing-remitting MS (RRMS) to secondary progressive MS (SPMS).

Multiple sclerosis (MS) is a chronic, inflammatory and neurodegenerative disease of the central nervous system (CNS) characterized by demyelination (i.e. loss of myelin proteins) and variable degrees of axonal loss and gliosis. The predominant opinion in neurology is that MS is an autoimmune disease. MS is the leading cause of neurological disability in young and middle-aged adults, affecting an estimated 2.5 million individuals worldwide. Most patients are diagnosed between the ages of 20 and 40 years (in a 2:1 female to male ratio).

Clinicians usually classify MS patients into different types of disease patterns, simply put generally MS is classified into 3 main clinical courses (phenotypes): Relapsing remitting MS (RRMS), Secondary progressive MS (SPMS) and Primary progressive MS (PPMS). Some of these types are related, for example SPMS results from a progression of MS from RRMS, such related types of MS are also referred to as states of MS or phases of MS or courses of MS or subforms of MS. RRMS and SPMS are generally recognized as part of the MS disease continuum. In clinical trials, MS-related disability may be measured in the Kurtzke Expanded Disability Status Scale (EDSS).

About 85% of patients initially experience RRMS. RRMS is characterized by discrete motor, sensory, cerebellar or visual attacks that occur over 1-2 weeks (relapse) and often resolve over 1-2 months (remission). A typical relapse frequency for RRMS patients is 0.8-1.2/year but can be highly variable. Although, during remission and relapse-free periods, the patient remains clinically stable, a substantial proportion of relapses (e.g. 42-57%) may result in incomplete recovery of function and thus may lead to permanent disability.

Within 10 years of RRMS onset, about half of RRMS patients develop secondary progressive MS. SPMS is characterized by gradually increasing disability independent of relapses. SPMS can be with or without relapses. which is characterized by a chronic and steady increase in physical and cognitive disabilities independent of relapses. SPMS is commonly defined as gradual progression of disability independent of relapses over at least 6 to 12 months following an initial RRMS state.

Typically, SPMS starts as SPMS with relapses, but as disability progresses, relapses disappear. PPMS has a progressive disease course from onset without any relapses or remissions. PPMS affects a smaller number of MS patients.

Thomson (2006) provides an overview on MS classifications (Thomson Core Evid. 1 (3):157-67 (2006)). Lublin (2014) provides—Recent Revised Lublin Criteria—that are also used to define MS phenotypes considering two aspects reflecting inflammatory or neurodegenerative processes, e.g. disease activity (based on clinical relapse rate and imaging findings) and disease progression—see Lublin, F. D. et al., “Defining the clinical course of multiple sclerosis”, Neurology, 1996, 46: 907-911 and Lublin, et al., “Defining the clinical course of multiple sclerosis: the 2013 revisions”, Neurology, 2014, 83: 278-86. Hence patients are described, as having (1) relapsing MS that is active (determined by clinical relapse and/or MRI activity) or inactive, with or without worsening of disability or (2) progressive MS, primary or secondary progressive disease that is active or inactive, with or without disability progression.

The disease (i.e. MS in RRMS state) in the patient subsequently may evolve or progress over a variable period of time into SPMS. This progression from RRMS to SPMS shows a huge variation—for example after 6 to 10 years from MS disease onset, approximately 25%-40% of patients with RRMS state have been reported to convert to SPMS state, with a median time to transition ranging between 10-23 years. Even when in SPMS state, which generally is characterized by increased disabilities independent of relapses, the MS patient can continue to experience relapses followed by complete or incomplete remission, generally at onset of SPMS i.e. in early stages of SPMS, which makes it difficult for physicians to understand if the patient is in RRMS state or has transitioned into SPMS state. Presently, the mechanism of transition from RRMS to SPMS is not clearly understood, and there are no reliable diagnostic tests or biomarkers to determine or predict this transition. Since MS is a complex disease, it is difficult, even for health professionals or physicians, to spot the changes in the patient that signal onset of SPMS.

Furthermore, the transition from RRMS to SPMS is a gradual process. The physician, even when presented with changes/symptoms in the patient, is not able to determine or predict with any certainty that the transition from RRMS to SPMS has occurred or is underway. The time in clinical practice required to re-classify RRMS patients who have clinically transitioned to SPMS, i.e. to identify that MS is in SPMS state, has been uncertain and long, and the period of uncertainty has been reported to be approximately up to 3 to 4 years (Katz et. al. Mult. Scler. J. 20 (12): 1654-7 (2014)). Different physicians rely on different symptoms to make a subjective assessment of the onset of SPMS. If a RRMS patient's symptoms are worsening, the physician is unable to predict or determine whether the worsening is left over from the last relapse (meaning the RRMS is continuing) or whether the RRMS is transitioning or has transitioned to SPMS. Different physicians use differing strategies, rely on different symptoms exhibited by the patient, various neurologic examinations, and repeat magnetic resonance imaging (MRI) scans to determine whether the transition from RRMS to SPMS has occurred. Furthermore, the subjectivity of an individual physician, resulting from his or her different training and different experience as compared to other physicians, weighs in heavy in the determination reached by that physician, and consequently different physicians often reach different conclusions as to whether the MS has progressed into SPMS or not, especially during the transitioning period of MS from RRMS to SPMS or during early phases of SPMS. As a result, not only different physicians often reach different conclusions regarding the progression of RRMS to SPMS, but also conclusive determination is often delayed which inadvertently results into delay in providing to the MS patient a regimen suitable for SPMS. Thomson (2006) also provides different therapy options for MS patients depending on the type of MS or state of MS.

Therefore, there exists a need for a technique to aid physicians in determining a state of MS, for example RRMS state or SPMS state. Such a technique would aid physicians in determining MS disease progression in a MS patient from RRMS to SPMS. The technique is required to be objective, i.e. devoid of the administering physician's subjectivity, fast and simple to use for physicians. The technique is also desired to be substantially comprehensive. Accurate early identification of SPMS in a MS patient can lead to optimal disease management and/or the most appropriate treatments options which will result in better long-term outcomes for the SPMS affected patient population. The state of MS in a patient is important for treatment decisions, such as determining a type of MS therapeutic that is suitable for the patient, for example if the patient is in a first state, say RRMS, the patient may be administered a first type of MS therapeutic whereas when the same patient has advanced to a second state, say SPMS, the patient may be administered a second type of MS therapeutic which is more suitable for the second state of MS.

An embodiment of the present disclosure provides an objective, fast and simple technique for determining a state of MS in a MS patient, such as MS disease progression in a patient from RRMS to SPMS.

The above-mentioned object is achieved by the subject-matter of the independent claims. Further embodiments of the present invention are subject-matters of the dependent claims.

“SPMS” is defined as “initial relapsing remitting disease course followed by progression with or without occasional relapses, minor remissions, and plateaus”, see Lublin (2014).

The diagnosis of MS with initial relapsing remitting disease course is defined by the 2010 Revised McDonald criteria, see Polman et al. “Diagnostic criteria for multiple sclerosis: 2010 revisions to the McDonald criteria”, Ann. Neurol., 2011; 69: 292-302.

Progression denotes the continuous worsening of neurological impairment over at last 6 months, see Rovaris. et al. “Secondary progressive multiple sclerosis: current knowledge and future challenges”; Lancet Neurology, 2006, 5: 343-354.

For the avoidance of doubt, it is hereby stated that the information disclosed earlier in this specification under the heading “Background” is relevant to the invention and is to be read as part of the disclosure of the invention.

In aspects of the present technique presented hereinafter a state of Multiple Sclerosis (MS) in a MS patient is determined. For example, in one embodiment, the state may be a first state i.e. Relapsing-Remitting Multiple Sclerosis (RRMS) or a second state i.e. Secondary Progressive Multiple Sclerosis (SPMS), and thus the method for determining the state of MS may be a method of determining disease progression in a MS patient from RRMS to SPMS.

Alternatively, in another embodiment, there may be three stages of MS that the present technique can determine—‘RRMS’ state, ‘in-transition from RRMS to SPMS’ state, and SPMS' state—accordingly in one embodiment of the present technique, the first state may be

‘RRMS’ state, and the second state may be one of ‘in-transition from RRMS to SPMS’ state and SPMS' state; whereas in another embodiment of the present technique the first state may be ‘RRMS’ state or ‘in-transition from RRMS to SPMS’ state, and the second state may be SPMS' state.

In one embodiment of the present technique, the first state may be one of a ‘RRMS’ state, a ‘in-transition from RRMS to SPMS’ state, and a combination thereof (i.e. a state that includes both ‘RRMS’ state and ‘in-transition from RRMS to SPMS’ state), and the second state may be SPMS' state. In another embodiment of the present technique, the first state may be ‘RRMS’ state and the second state may be one of a SPMS' state, a ‘in-transition from RRMS to SPMS’ state, and a combination thereof (i.e. a state that includes both SPMS' state and ‘in-transition from RRMS to SPMS’ state).

In yet another embodiment, the present technique determines whether the state of MS in the patient is a first state, a second state or a third state, wherein the first state is ‘RRMS’ state, the second state is SPMS' state and the third state is ‘in-transition from RRMS to SPMS’ state.

In the present technique, the phrase ‘determining a state of MS’ and like phrases can refer to, for example, ‘predicting a state of MS’ or ‘assessing a state of MS’ or ‘identifying a state of MS’ or ‘recognizing a state of MS’.

In a first aspect of the present technique a method for determining a state of Multiple Sclerosis (MS) in a MS patient is presented. In an embodiment of the method, in a first step a graphical user interface (GUI) is caused, by one or more processors, to be output onto a display. The graphical user interface comprises: a field for displaying patient data queries and a field for displaying age query. The ‘field’ can refer to ‘an area on a displayed page’. When GUI is displayed, the fields for displaying patient data queries and age query include the patient data queries and age query, respectively.

The patient data queries comprise a plurality of items grouped into a first group, a second group and a third group. The first group comprises one or more items relating to relapse and recovery of the patient in a first predetermined period. Optionally, the first group may include MRI inflammatory disease activity. The second group comprises one or more items relating to symptoms experienced by the patient in a second predetermined period. The third group comprises one or more items relating to impacts due to MS experienced by the patient in a third predetermined period. In an embodiment of the invention, each ‘item’ is a patient data query. Queries may pertain to either relapse and recovery of the patient in the first predetermined period, or symptoms experienced by the patient in the second predetermined period or impacts due to MS experienced by the patient in the third predetermined period. In an embodiment of the invention, each of the first, the second and the third predetermined periods are established or decided in advance i.e. before the GUI is used for inputting the responses. In embodiments of the invention, each of the first, the second and third predetermined periods are of same length, and may refer to the same period, e.g. a period of six months ending on a predetermined date for example on a date when the method of the present technique is being used. The predetermined period may be a period shorter or longer than six months for example one month, or two months, or three months, or four months, or five months, or six months, or seven months, or eight months or nine months.

The phrase ‘predetermined’, as used in the present technique, can refer to, for example, ‘established or decided in advance’ before the method of the present technique is being performed by a user, for example by a health care provider or a physician. For example, the phrase ‘assigned’ as used in the present technique can refer to ‘allocated’.

Each item of the plurality of items has an assigned predetermined weight and comprises a plurality of corresponding predetermined responses. Each response of the corresponding predetermined responses, i.e. each of the responses for a corresponding item, has an assigned predetermined score. Each response of the corresponding predetermined responses is indicative of a distinct information pertaining to the corresponding item. All the responses to a given item together represent the different possible conditions a patient could be in. For example, if the item is a query such as ‘Pain experienced?’, the responses may be ‘Yes’ and ‘No’ (For the given item—‘Pain experienced?’—all the responses together i.e. ‘Yes’ and ‘No’ together represent the different possible conditions the patient could be in, and furthermore each response represents a distinct information pertaining to the corresponding item i.e. ‘Yes’ represents the distinct information that pain is or has been felt, whereas ‘No’ represents the distinct information that pain is not or has not been felt whereas the responses together present all the different possible responses to that item). The predetermined responses are presented as choices for possible answers pertaining to that item. In the present technique, the items and their corresponding responses may be displayed together, thus a user sees the query and the possible responses, to the query, from which the user could select.

In the method, in one embodiment, the different fields, i.e. the fields displaying patient data queries and the age query, of the GUI may be displayed simultaneously i.e. together in the same page. In another embodiment, the different fields, i.e. the fields displaying patient data queries and the age query, of the GUI may be displayed sequentially i.e. first one of the field displaying patient data queries and the field displaying age query is displayed in one or more pages, i.e. say one or more first pages, and thereafter the other field, from the field displaying patient data queries and the field displaying age query, is displayed on one or more subsequent pages, say one or more second pages, displayed after the one or more first pages.

In the method, after the GUI is displayed, i.e. after the field having patient data queries is displayed, in a second step the responses to the patient data queries are inputted by selecting at least one response, e.g. only one response, from the plurality of corresponding predetermined responses for each of the plurality of items displayed in the GUI. The responses for the items are inputted by a user such as physician for example by selecting one or more of the displayed responses for a given item, for e.g. by selecting only one response from of the displayed responses for a given item. The responses are inputted to the one or more processors to be used for processing of the responses.

Thereafter, in the method, in a third step an item score is determined, by the one or more processors, for each of the plurality of items. The item scores are determined or calculated based on the weight of the item and the score of the selected response for the item. Alternatively, if a plurality of responses are selected or inputted for a given item, then the item score for that item is determined or calculated based on the weight of the item and the scores of the selected responses for that item, for example, but not limited to, by using an average of the scores of the selected responses or for example by using the highest score from the scores of the selected responses. In one embodiment, the item score for a given item may be determined by multiplying the weight of the item and the score of the selected response for the item, when only one response is selected for that item. Alternatively, the item score for a given item may be determined by multiplying the weight of the item and the average of the scores of the selected responses for the item, when more than one response is selected for that item. In yet another alternative embodiment, the item score for a given item may be determined by multiplying the weight of the item and the highest of the scores of the selected responses for the item, when more than one response is selected for that item.

Subsequently, in the method, in a fourth step after the third step, a first group score, a second group score and a third group score are determined by the one or more processors. The first, the second and the third group scores are determined or calculated based on the item scores of the items of the first, the second and the third groups, respectively. In an embodiment, the first group score, the second group score and the third group score are determined by adding the item scores of the items comprised in the first group, the second group and the third group, respectively. In another embodiment, the first group score, the second group score and the third group score are determined by weighted averaging of the item scores of the items comprised in the first group, the second group and the third group, respectively.

In the method, in a fifth step an age of the patient (i.e. the age of the patient in years or years and month or similar format) is inputted as a response to the age query in the GUI. The age of the patient is allocated to one of a plurality of predetermined age groups, wherein each of the predetermined age groups has an assigned predetermined score. The age of the patient is inputted by a user such as physician for example by selecting one of the displayed age groups to which the patient's age belongs i.e. age of the patient is allocated by the user to one of the age groups from the plurality of predetermined age groups displayed in the GUI. Alternatively, a user may input the age of the patient simply by inputting a numerical value reflecting the age of the patient, and thereafter the inputted age of the patient is allocated, by the one or more processors, to one age group from the plurality of predetermined age groups displayed in the GUI. The age of the patient is inputted to the one or more processors to be used for processing of the age. After the age is inputted, in the method of the present technique, an age score is determined by the one or more processors. The age score is determined or calculated based on the score of the allocated age group and a predetermined weight assigned for the age query. In an embodiment, the age score is determined by multiplying the score of the allocated age group and the predetermined weight assigned for the age.

Subsequently, in a sixth step of the method a total score (TOTAL) based on the first group score, the second group score, the third group score and the age score is generated. The total score is generated i.e. determined or calculated by the one or more processors. In another embodiment, the total score is generated by adding the first group score, the second group score, the third group score, and the age score.

Thereafter, in a seventh step of the method the one or more processors determines the state of MS in the MS patient, wherein the state may either be a first state or a second state. For example, in one embodiment, the first state may be RRMS and/or the second state may be SPMS. In yet another embodiment, when the first state is RRMS and the second state is SPMS, the determining the state of MS comprises determining a status of progression of MS from RRMS to SPMS based on the total score, or in other words the one or more processors determines or calculates or predicts if the MS patient is still in RRMS state or if the MS patient has progressed from the RRMS state to the SPMS state, or if the MS patient is in transition i.e. progressing from RRMS state to SPMS state i.e. is in ‘in-transition’ state.

In one embodiment of the seventh step, the first state may be the ‘RRMS’ state, and the second state may be one of the ‘in-transition from RRMS to SPMS’ state and the SPMS' state; whereas in another embodiment of the present technique the first state may be the ‘RRMS’ state or the ‘in-transition from RRMS to SPMS’ state, and the second state may be the SPMS' state.

In one embodiment of the present technique, the first state may be one of the ‘RRMS’ state, the ‘in-transition from RRMS to SPMS’ state, and a combination thereof (i.e. a state that includes both ‘RRMS’ state and ‘in-transition from RRMS to SPMS’ state), and the second state may be the SPMS' state. In another embodiment of the present technique, the first state may be ‘RRMS’ state and the second state may be one of the SPMS' state, the ‘in-transition from RRMS to SPMS’ state, and a combination thereof (i.e. the state that includes both SPMS' state and ‘in-transition from RRMS to SPMS’ state).

In yet another embodiment, the present technique determines whether the state of MS in the patient is a first state, a second state or a third state, wherein the first state is ‘RRMS’ state, the second state is SPMS' state and the third state is ‘in-transition from RRMS to SPMS’ state.

Finally, in an eighth step of the method the state of the MS so determined is outputted to the user by an output means. In the embodiment in which the first state is RRMS state and the second state is SPMS state, the state of the MS so determined, i.e. indication showing RRMS state or SPMS state as determined, is outputted to the user by an output means. In another embodiment, in which the first state is RRMS state and the second state is SPMS state, and in which the status of progression of MS from RRMS to SPMS is determined based on the total score, the status of progression of MS from RRMS to SPMS so determined is outputted to the user by an output means. In other words, the output means transmits information to the user that the MS is still in RRMS state or that the MS is in SPMS or that MS is in transition progressing from RRMS to SPMS i.e. is in ‘in-transition from RRMS to SPMS’ state. The outputting of the state of MS or status of progression of MS from RRMS to SPMS is provided by one of a visual output, an audio output, a tactile output, and a combination thereof.

It may be noted that the above-mentioned second step of inputting the responses to the patient data queries and the fifth step of inputting the response to the age query may be performed in the order in which the fields displaying the patient data queries and the age query are displayed in the first step of the method. For example, in an embodiment, the field displaying patient data queries and the field displaying the age query may be displayed simultaneously and therefore in this embodiment, the second step and the fifth step may be simultaneously performed i.e. responses can be inputted together in the same page. In another embodiment, in the first step of the method, the field displaying patient data queries may be displayed on one or more first pages and the second step is performed, thereafter the field displaying the age query may be displayed on a second page and then the fifth step is performed. In yet another embodiment, in the first step of the method, the field displaying the age query may be displayed on a first page and the fifth step is performed, thereafter the field displaying patient data queries may be displayed on one or more second page and then the second step is performed.

In another embodiment of the method, in the first step, the GUI, in addition to the fields for displaying patient data queries and age query, comprises a field for displaying Expanded Disability Status Scale (EDSS) query and/or a field for displaying Timed 25-Foot Walk (T25FW) query. The GUI may be used for inputting of responses to the patient data queries and age query, and optionally to the EDSS query and/or T25FW query. When displayed, the fields for displaying EDSS and/or T25FW queries include the EDSS and/or T25FW query, respectively.

In an embodiment of the method, in a ninth step an EDSS score and/or T25FW score of the patient is inputted as a response to the EDSS query and/or T25FW query in the GUI. The EDSS score and/or T25FW score of the patient is inputted by a user such as physician for example by selecting one from a plurality of the displayed possible EDSS scores and/or T25FW scores or for example by simply inputting the numerical value reflecting the EDSS score and/or T25FW score of the patient as was previously determined. The EDSS score and/or T25FW score of the patient is inputted to the one or more processors to be used for processing of the EDSS score and/or T25FW score. The EDSS is a method for quantifying disability in MS and monitoring changes in the level of disability over time. The EDSS scale ranges from 0 to 10, in increments of 0.5, unit that represent higher levels of disability. EDSS scoring (i.e. determining the EDSS score) is based on an examination of the MS patient by a neurologist which is performed in advance of the use of the present method. The T25FW is a quantitative mobility and leg function performance test based on a timed 25-walk. The T25FW is administered to the MS patient in person by a trained examiner and the T25FW score is determined. The EDSS and the T25FW is a well-known quantitative mobility and leg function performance test based on a timed 25-walk, and hence has not been described in further details herein for sake of brevity. The EDSS and/or T25FW scores are determined in advance of the use of the present method.

It may be noted that the above-mentioned ninth step of inputting the responses to the EDSS query and/or T25FW query may be performed in the order in which the fields displaying the patient data queries, the age query, and the EDSS query and/or T25FW query are displayed in the first step of the method. For example, in an embodiment, the fields displaying patient data queries, the age query and the field displaying the EDSS query and/or T25FW query may be displayed simultaneously and therefore in this embodiment the second step, the fifth step and the ninth step may be simultaneously performed i.e. responses can be inputted together in the same page. In another embodiment, the field displaying the EDSS query and/or T25FW query may be displayed in the same page as one of the remaining two fields i.e. the fields displaying the patient data queries and the age query, before or after the other of the remaining two fields is displayed on another page and therefore in this embodiment, the ninth step is performed simultaneously with one of the second or fifth steps, and before or after the other of the second and fifth steps. For example in one embodiment, the field displaying the EDSS query and/or T25FW query may be displayed in the same page as the field displaying the age query, before or after the field displaying the patient data queries is displayed on another page and therefore in this embodiment, the ninth step is performed simultaneously with the fifth step, and before or after the second step.

In an embodiment of the method, after the EDSS score and/or T25FW is inputted in the ninth step, in a tenth step of the method of the present technique, an EDSS group score and/or T25FW group score is determined by the one or more processors.

The EDSS group score is determined or calculated based on the inputted EDSS score and a predetermined weight assigned for the EDSS query. In one embodiment, the EDSS group score is determined by acquiring the EDSS score for the patient, generating a weighted EDSS score from the acquired EDSS score, and generating a reweighted EDSS score by multiplying the weighted EDSS score and the predetermined weight for the EDSS score. The weighted EDSS score may be generated by expressing the acquired EDSS score as a fraction of the maximum possible score of the Expanded Disability Status Scale e.g. the maximum possible score of the EDSS scale used is ‘10’.

In the present technique, in an embodiment, instead of using the EDSS score, the T25FW may be used, for example when the EDSS score is unavailable. Alternatively, in an embodiment, both the EDSS score and the T25FW may be used.

Subsequently, in an embodiment of the method in which the ninth and the tenth steps are performed, the total score is generated in the sixth step based on the first group score, the second group score, the third group score, the EDSS group score and/or T25FW group score, and the age score is generated. The total score is generated i.e. determined or calculated by the one or more processors. In another embodiment, the total score is generated by adding the first group score, the second group score, the third group score, the age score and the EDSS group score and/or T25FW group score.

It may be noted that, for sake of simplicity, in the following description generally ‘EDSS score’, ‘EDSS query’, ‘EDSS group score’ etc. have been used, however, it may be noted that instead of EDSS score the T25FW score or results can also be used.

In a second aspect of the present technique a computer implemented method for determining a state of Multiple Sclerosis (MS) in a MS patient is presented. The computer implemented method comprises steps as described hereinabove for the first aspect of the present technique.

In a third aspect of the present technique, one or more non-transitory computer-readable media storing computer-executable instructions that, when executed by one or more processors, cause the one or more processors to perform a method according to the afore-mentioned first aspect of the present technique is presented.

In a fourth aspect of the present technique, an apparatus for determining a state of Multiple Sclerosis (MS) in a MS patient is presented. For example, the state may be a first state i.e. RRMS or a second state i.e. SPMS, and thus the apparatus for determining the state of MS may be an apparatus for determining disease progression in a MS patient from RRMS to SPMS. The apparatus comprises one or more processors to execute a method according to the afore-mentioned first aspect of the present technique, a display for outputting the graphical user interface, and an output means for outputting the state of the MS. In one embodiment, the state of MS is a status of progression of MS from RRMS to SPMS so determined by the one or more processors.

In a fifth aspect of the present technique a system for determining a state of Multiple Sclerosis (MS) in a MS patient is presented. The state may be either a first state or a second state. In another embodiment the state may be either a first state or a second state or a third state.

The system comprises a display configured to output a graphical user interface (GUI) to a user. The GUI includes fields for displaying patient data queries and age query for inputting of responses to the patient data queries and the age query. When displayed, the fields for displaying patient data queries and age query include the patient data queries and age query, respectively.

The patient data queries comprise a plurality of items grouped into a first group, a second group and a third group, wherein the first group comprises at least one item relating to relapse and recovery of the patient in a first predetermined period, the second group comprises at least one item relating to symptoms experienced by the patient in a second predetermined period and the third group comprises at least one item relating to impacts experienced by the patient in a third predetermined period. Each item of the plurality of items has an assigned predetermined weight and comprises a plurality of corresponding predetermined responses, and wherein each response of the corresponding predetermined responses has an assigned predetermined score and is indicative of a distinct information pertaining to the corresponding item. The system also includes a user interface for inputting of responses to the patient data queries and for inputting of an age of the patient as a response to the age query, wherein at least one response from the plurality of corresponding predetermined responses for each of the plurality of items displayed in the GUI is selectable for inputting of the responses to the patient data queries. The system further includes one or more processors configured to receive the responses to the patient data queries and the age query and to: determine an item score for each of the plurality of items based on the weight of the item and the score of the selected response for the item; determine a first group score, a second group score and a third group score based on the item scores of the items of the first group, the second group and the third group, respectively; allocate an inputted age of the patient to one of a plurality of predetermined age groups, wherein each of the predetermined age groups has an assigned predetermined score; determine an age score based on the score of the allocated age group and a predetermined weight assigned for the age query; generate a total score based on the first group score, the second group score, the third group score and the age score; determine the state of MS in the MS patient based on the total score, wherein the state is either a first state of MS or a second state of MS; and output the state of MS so determined. Furthermore, the system includes an output means for indicating the state of MS so determined by the one or more processors.

In an embodiment, the first state is RRMS. In yet another embodiment, the second state is SPMS. In a further embodiment, the first state is RRMS and the second state is SPMS. In a further embodiment, the first state is RRMS and the second state is SPMS and the processor is configured to determine a status of progression of MS from RRMS to SPMS based on the total score. Furthermore, the output means is configured to indicate the state of MS or the status of progression of MS so determined by the one or more processors.

In one embodiment of the system, the first state may be the ‘RRMS’ state, and the second state may be one of the ‘in-transition from RRMS to SPMS’ state and the SPMS' state; whereas in another embodiment of the system the first state may be the ‘RRMS’ state or the ‘in-transition from RRMS to SPMS’ state, and the second state may be the SPMS' state.

In one embodiment of the system, the first state may be one of the ‘RRMS’ state, the ‘in-transition from RRMS to SPMS’ state, and a combination thereof (i.e. a state that includes both ‘RRMS’ state and ‘in-transition from RRMS to SPMS’ state), and the second state may be the SPMS' state. In another embodiment of the system, the first state may be ‘RRMS’ state and the second state may be one of the SPMS' state, the ‘in-transition from RRMS to SPMS’ state, and a combination thereof (i.e. the state that includes both SPMS' state and ‘in-transition from RRMS to SPMS’ state).

In yet another embodiment, the system, i.e. one or more processors of the system, is configured to determine whether the state of MS in the patient is a first state, a second state or a third state, wherein the first state is ‘RRMS’ state, the second state is SPMS' state and the third state is ‘in-transition from RRMS to SPMS’ state.

In an embodiment of the system, the one or more processors is configured to determine the item score by multiplying the weight of the item and the score of the selected response for the item.

In another embodiment of the system, the one or more processors is configured to determine the first group score, the second group score and the third group score by adding the item scores of the items comprised in the first group, the second group and the third group, respectively. In another embodiment of the system, the one or more processors is configured to determine the first group score, the second group score and the third group score by weighted averaging of the item scores of the items comprised in the first group, the second group and the third group, respectively.

In another embodiment of the system, the one or more processors is configured to determine the age score by multiplying the score of the allocated age group and the predetermined weight assigned for the age.

In a further embodiment of the system, the first predetermined period, the second predetermined period and the third predetermined period are same.

In yet another embodiment of the system, each of the first predetermined period, the second predetermined period and the third predetermined period is six months from a predetermined date.

In an embodiment of the system, the GUI may also include, besides the fields for displaying the patient data queries and the age query, a field for displaying EDSS query and/or a field for displaying a T25FW query for inputting of responses to the EDSS query and/or the T25FW query, respectively. When displayed, the fields for displaying EDSS query and/or the T25FW include the EDSS query and/or the T25FW. The user interface of the system is configured for inputting of an EDSS score and/or a T25FW score of the patient as a response to the EDSS query and/or the T25FW query, respectively, in addition to inputting of responses to the patient data queries and the age query. The one or more processors of the system are configured to receive, in addition to the responses to the patient data queries and the age query, the responses to the EDSS query and/or the T25FW query. The one or more processors is further configured to determine an EDSS group score based on an inputted EDSS score and a predetermined weight assigned for the EDSS query and/or determine a T25FW group score based on an inputted T25FW score and a predetermined weight assigned for the T25FW query; and to generate a total score based on the first group score, the second group score, the third group score, the age score and at least one of the EDSS group score and the T25FW group score. In one embodiment of the system, the one or more processors is configured to generate the total score by adding the first group score, the second group score, the third group score, the age score and at least one of the EDSS group score and the T25FW group score.

In yet another embodiment of the system, the one or more processors is configured to determine the EDSS group score by: acquiring the EDSS score for the patient, generating a weighted EDSS score from the acquired EDSS score as a fraction of the maximum possible score of the Expanded Disability Status Scale, and generating a reweighted EDSS score by multiplying the weighted EDSS score and the predetermined weight for the EDSS score.

In a sixth aspect of the present technique, a method for treating multiple sclerosis (MS) in a patient in need thereof is presented. The method comprises a first step of determining in a patient the state of MS and a second step of administering to the patient an MS therapeutic based on the state so determined. In the method for treating MS, the determining the state is same as explained hereinabove for first aspect of the present technique.

In an embodiment according to the sixth aspect, the first state is RRMS and/or the second state is SPMS.

In one embodiment according to the sixth aspect, the first state may be the ‘RRMS’ state, and the second state may be one of the ‘in-transition from RRMS to SPMS’ state and the SPMS' state; whereas in another embodiment the first state may be the ‘RRMS’ state or the ‘in-transition from RRMS to SPMS’ state, and the second state may be the SPMS' state.

In one embodiment according to the sixth aspect, the first state may be one of the ‘RRMS’ state, the ‘in-transition from RRMS to SPMS’ state, and a combination thereof (i.e. a state that includes both ‘RRMS’ state and ‘in-transition from RRMS to SPMS’ state), and the second state may be the SPMS' state. In another embodiment according to the sixth aspect, the first state may be ‘RRMS’ state and the second state may be one of the SPMS' state, the ‘in-transition from RRMS to SPMS’ state, and a combination thereof (i.e. the state that includes both SPMS' state and ‘in-transition from RRMS to SPMS’ state).

In yet another embodiment according to the sixth aspect, determining in the patient the state of MS comprises whether the state of MS in the patient is a first state, a second state or a third state, wherein the first state is ‘RRMS’ state, the second state is SPMS' state and the third state is ‘in-transition from RRMS to SPMS’ state. In this embodiment an MS therapeutic based on the state so determined is administered to the patient.

The terms “treatment”/“treating” as used herein includes, but is not limited to: (1) preventing or delaying the appearance of clinical symptoms of the state of MS, associated disorder or condition developing in an animal, particularly a mammal and especially a human, that may be afflicted with or predisposed to the MS state, disorder or condition but does not yet experience or display clinical or subclinical symptoms of the state, disorder or condition; (2) inhibiting the state, disorder or condition (e.g. arresting, reducing or delaying the development of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

In an embodiment of the method for treating MS, the MS therapeutic is an sphingosine-1-phosphate (S1P) receptor modulator.

In another embodiment of the method for treating MS, the S1P receptor modulator is fingolimod or a pharmaceutically acceptable salt thereof. In a further embodiment of the method, the patient is administered fingolimod if the state of MS in the patient is the first state, for example the first state is RRMS.

In yet another embodiment of the method for treating MS, the S1P receptor modulator is siponimod or a pharmaceutically acceptable salt thereof. In a further embodiment of the method, the patient is administered siponimod if the state of MS in the patient is the second state, for example the second state is SPMS. In another embodiment of the method, the patient is administered siponimod if the state of MS in the patient is determined to be in the third state, wherein the third state is ‘in-transition from RRMS to SPMS’ state.

The phrases used in any of the second aspect, the third aspect, the fourth aspect, the fifth aspect and the sixth aspect of the present technique are same as like phrases used in the first aspect of the present technique and therefore may be understood as explained herein for the first aspect of the present technique. More specifically, the patient data queries, the plurality of items, the first group, the second group, the third group, the first, the second and third predetermined periods, assigned predetermined weight for each item, the plurality of corresponding predetermined responses, assigned predetermined score for each response, EDSS query, EDSS group score, weight assigned to the EDSS query, the age score, the age groups and their corresponding scores, weight assigned to the age score, inputting of responses to the patient data queries, inputting of the EDSS score of the patient as a response to the EDSS query and inputting of an age of the patient as a response to the age query and similar terms as used in any of the second, third, fourth, fifth or sixth aspects of the present technique are to be understood as explained hereinabove for the first aspect of the present technique.

Furthermore, in each aspect of the present technique, e.g. the first, the second, the third, the fourth, the fifth and the sixth aspects of the present technique, the items of the first group are selected from: an item indicating if the patient has experienced any relapses (Q1), an item indicating a number of relapses the patient has experienced (Q2), an item indicating an extent of recovery of the patient from a last relapse (Q3), an item indicating if a Magnetic Resonance Imaging, MRI, has been performed on the patient (Q4), and an item indicating if the performed MRI showed new signs of activity related to MS (Q5). One or more of the aforementioned items, Q1 to Q5, may be selected for example, in one embodiment the first group includes one of Q1 to Q5, whereas in another embodiment the first group includes two of Q1 to Q5, whereas in yet another embodiment the first group includes three of Q1 to Q5, whereas in yet another embodiment the first group includes four of Q1 to Q5, and in yet another embodiment the first group includes all of Q1 to Q5.

Also, in each aspect of the present technique, the items of the second group are selected from: an item indicating visual symptoms related to MS (Q6), an item indicating motor symptoms related to MS (Q7), an item indicating ambulatory symptoms related to MS (Q8), an item indicating coordination and balance symptoms related to MS (Q9), an item indicating pain experienced due to MS (Q10), an item indicating sensory symptoms related to MS (Q11), an item indicating bladder and bowel symptoms related to MS (Q12), an item indicating speech symptoms related to MS (Q13), an item indicating cognitive symptoms related to MS (Q14), and an item indicating fatigue symptoms related to MS (Q15). One or more of the aforementioned items, Q6 to Q15 may be selected for example, in one embodiment the second group includes one of Q6 to Q15, in another embodiment the second group includes two of Q6 to Q15, in yet another embodiment the second group includes three of Q6 to Q15, in yet another embodiment the second group includes four of Q6 to Q15, in another embodiment the second group includes five of Q6 to Q15, in yet another embodiment the second group includes six of Q6 to Q15, in yet another embodiment the second group includes seven of Q6 to Q15, in another embodiment the second group includes eight of Q6 to Q15, in yet another embodiment the second group includes nine of Q6 to Q15, and in yet another embodiment the second group includes all of Q6 to Q15.

Also, in each aspect of the present technique, the items of the third group are selected from: an item indicating impact on mobility of the patient due to MS (Q16), an item indicating impact on self-care of the patient due to MS (Q17), an item indicating impact on daily activities of the patient due to MS (Q18), an item indicating impact on hobbies and leisure time of the patient due to MS (Q19), and an item indicating impact on paid and unpaid work of the patient due to MS (Q20). One or more of the aforementioned items, Q16 to Q20, may be selected for example, in one embodiment the third group includes one of Q16 to Q20, whereas in another embodiment the third group includes two of Q16 to Q20, whereas in yet another embodiment the third group includes three of Q16 to Q20, whereas in yet another embodiment the third group includes four of Q16 to Q20, and in yet another embodiment the third group includes all of Q16 to Q20.

In each aspect of the present technique, the GUI may include—one or more of items selected, as explained hereinabove, from Q1 to Q5, and one or more of items selected, as explained hereinabove, from Q6 to Q15, and one or more of items selected, as explained hereinabove, from Q16 to Q20, to form the first group, the second group and the third group, respectively.

Furthermore, an embodiment according to each aspect of the present technique, includes the first group comprising all the items from Q1 to Q5, the second group comprising all the items from Q6 to Q15 and the third group comprising all the items from Q16 to Q20.

The advantage of the present technique is that the technique is objective since it does not rely on the subjective assessment of the physician, fast and simple to use. The technique is substantially comprehensive as the technique includes in reaching the determination: one or more items relating to relapse and recovery of the patient, one or more items relating to symptoms experienced by the patient, one or more items relating to impacts experienced by the patient, and the age of the patient. Additionally, embodiments of the technique that use one or more items relating to relapse and recovery of the patient, one or more items relating to symptoms experienced by the patient, one or more items relating to impacts experienced by the patient, the age of the patient and at least one of the EDSS score and the T25FW score are even more comprehensive. The technique reduced or obviates subjectivity of a user such as a physician or a health care provider in assessing the state of MS in the patient, for example in assessing a progression of RRMS to SPMS, and instead provides the user an objective tool for determining the state of MS disease or the progression from RRMS to SPMS. The present technique leads to accurate early identification of SPMS patients which consequently leads to the most appropriate management of the disease and treatment options that will result in better long-term outcomes for the SPMS affected patient population.

The above-mentioned attributes and other features and advantages of the present technique and the manner of attaining them will become more apparent and the present technique itself will be better understood by reference to the following description of embodiments of the present technique taken in conjunction with the accompanying drawings, wherein:

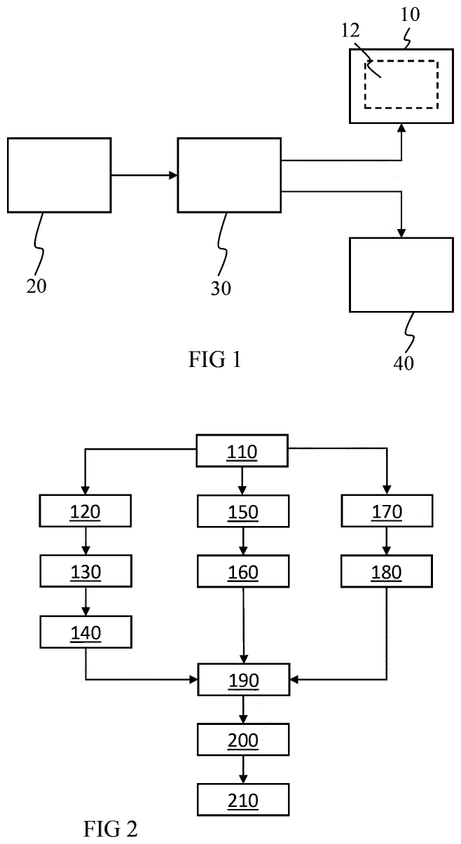

FIG. 1 schematically illustrates an embodiment of a system for determining a state of MS according to the present technique;

FIG. 2 is a flow chart depicting an embodiment of a method for determining a state of MS according to the present technique;

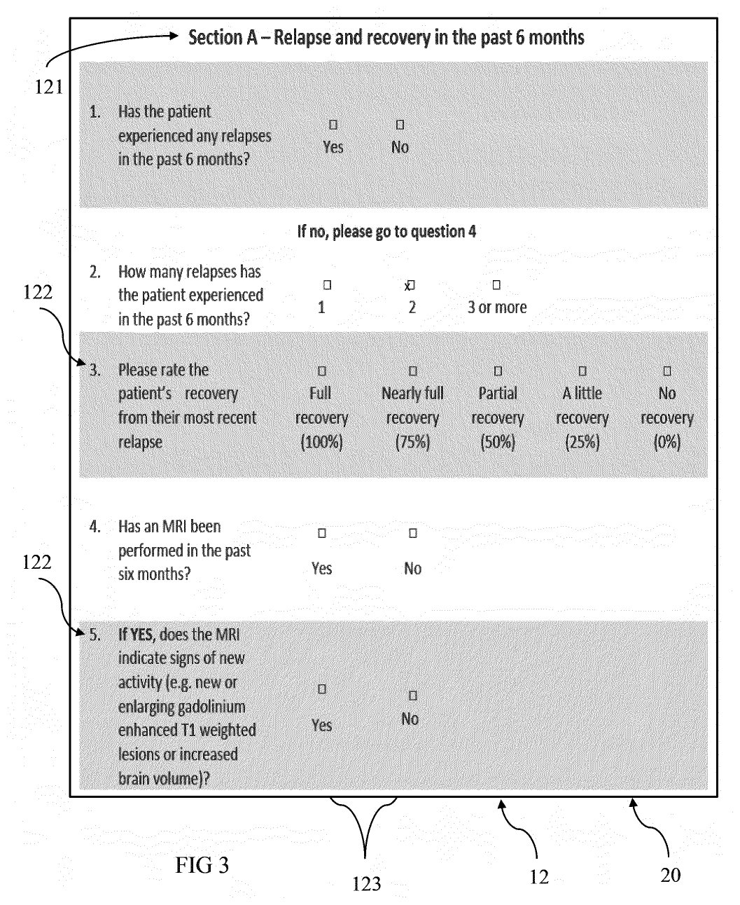

FIG. 3 is a screenshot of a graphical user interface (GUI) of the present technique illustrating items grouped into a first group along with corresponding predetermined responses for each item;

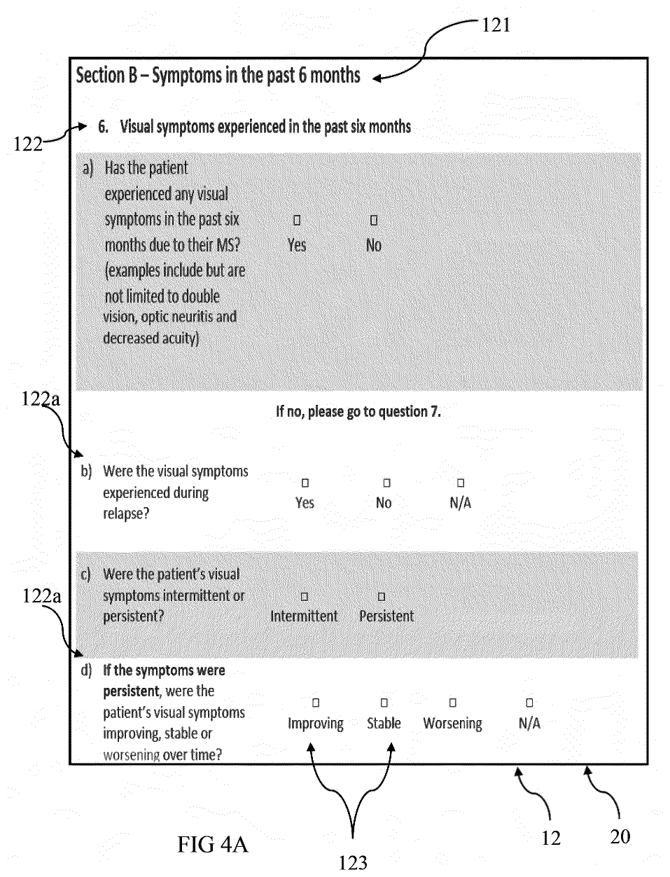

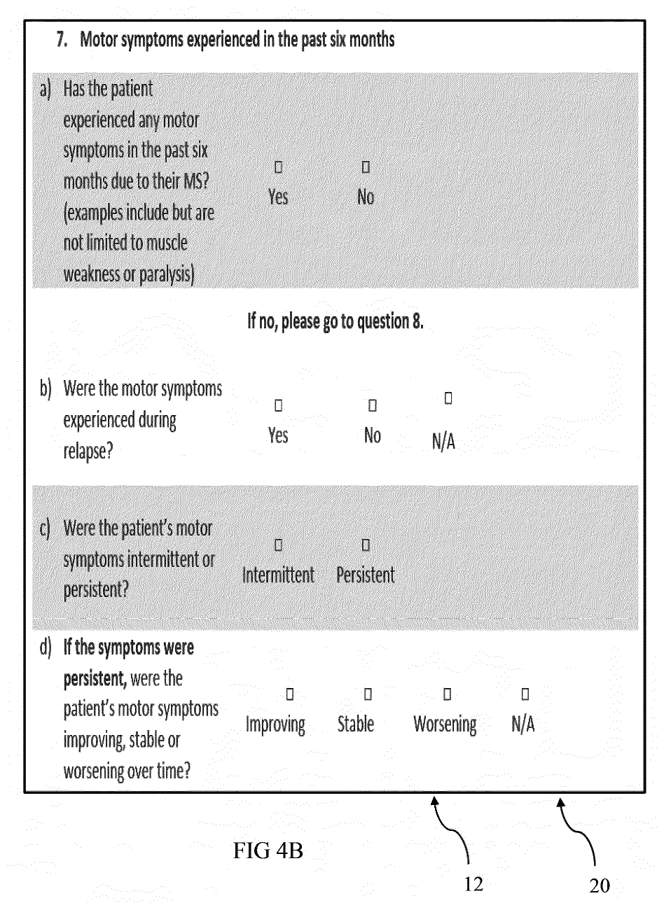

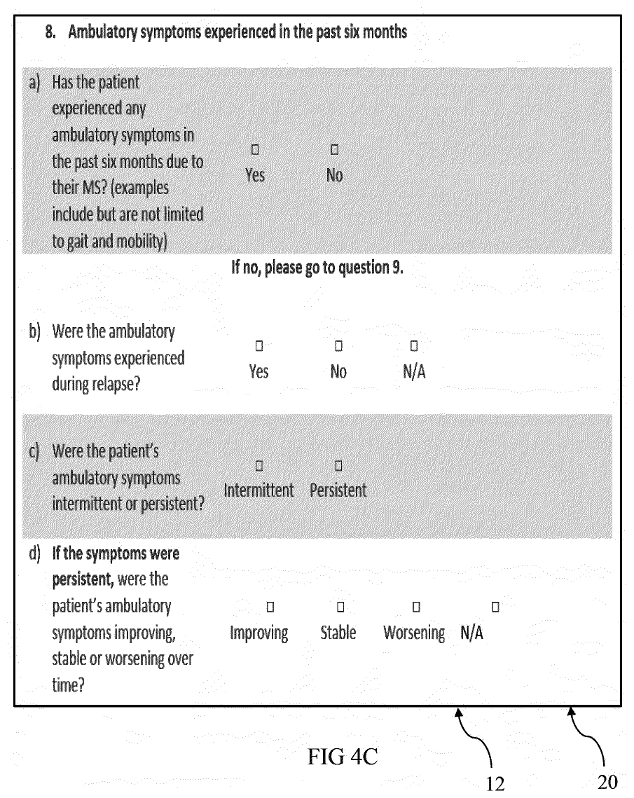

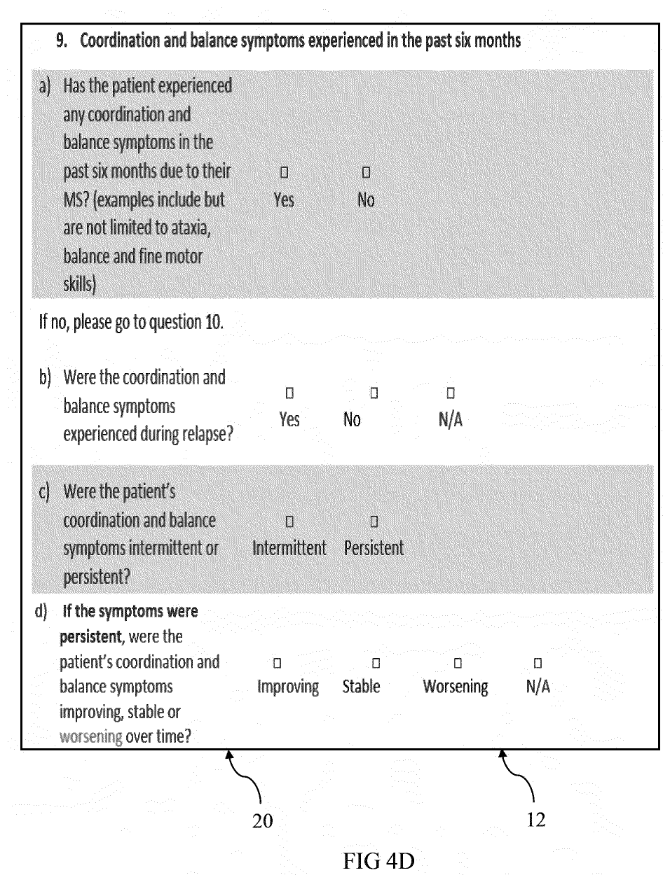

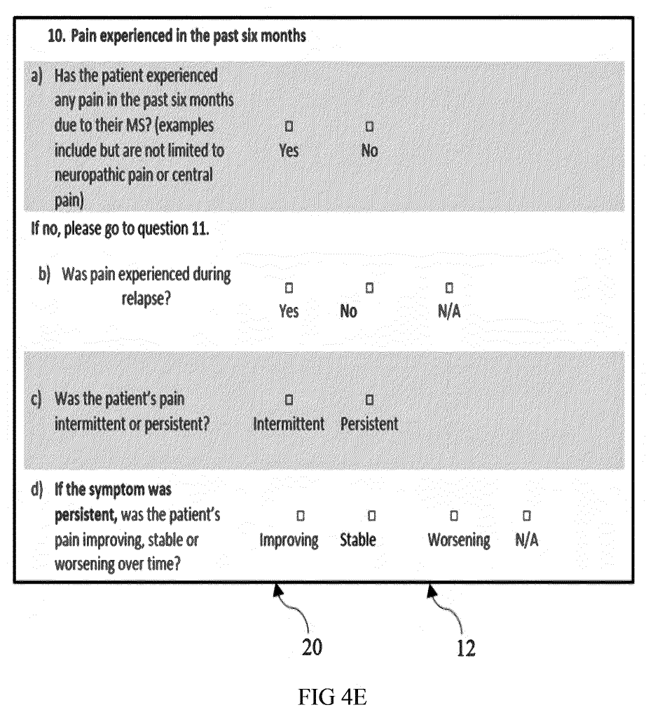

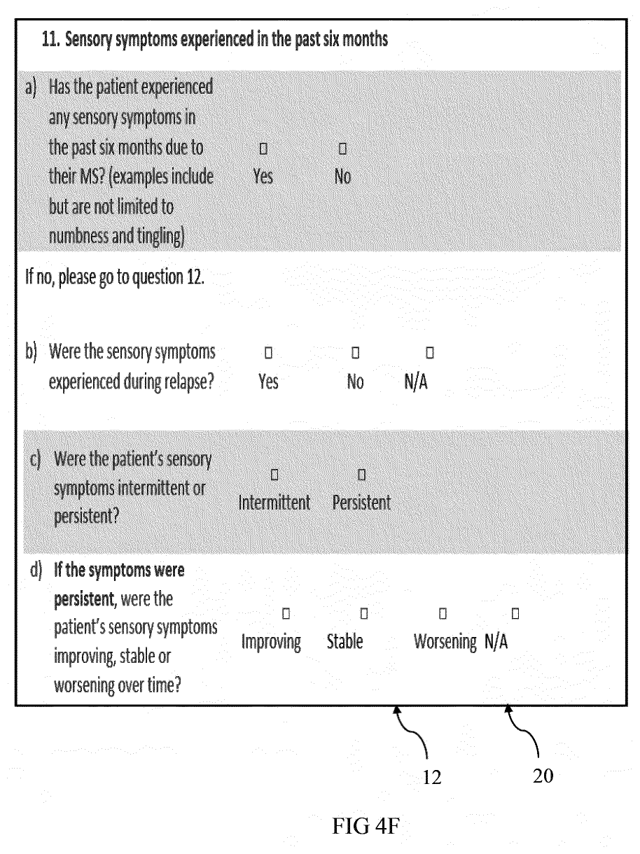

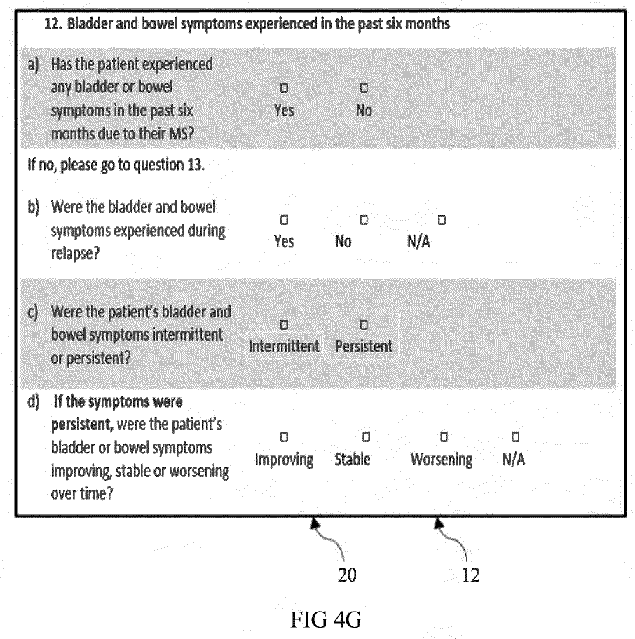

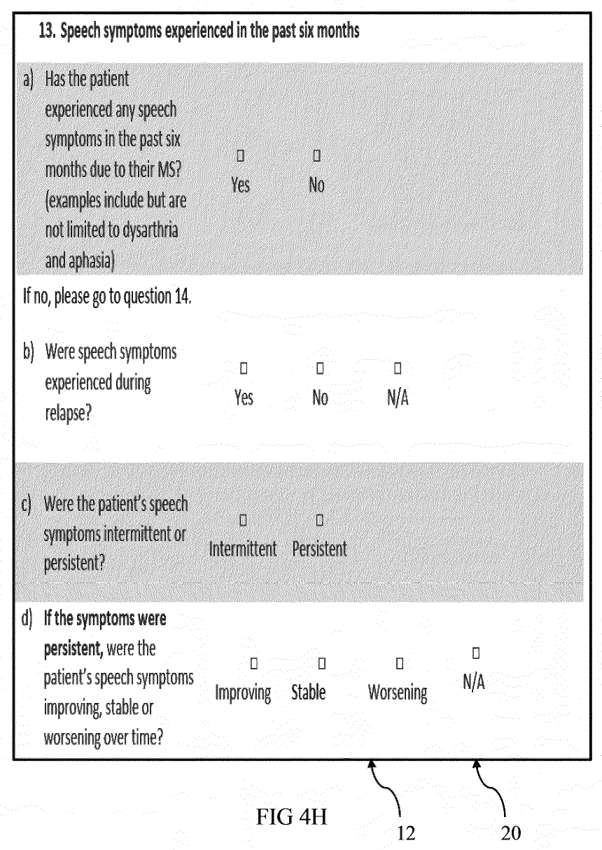

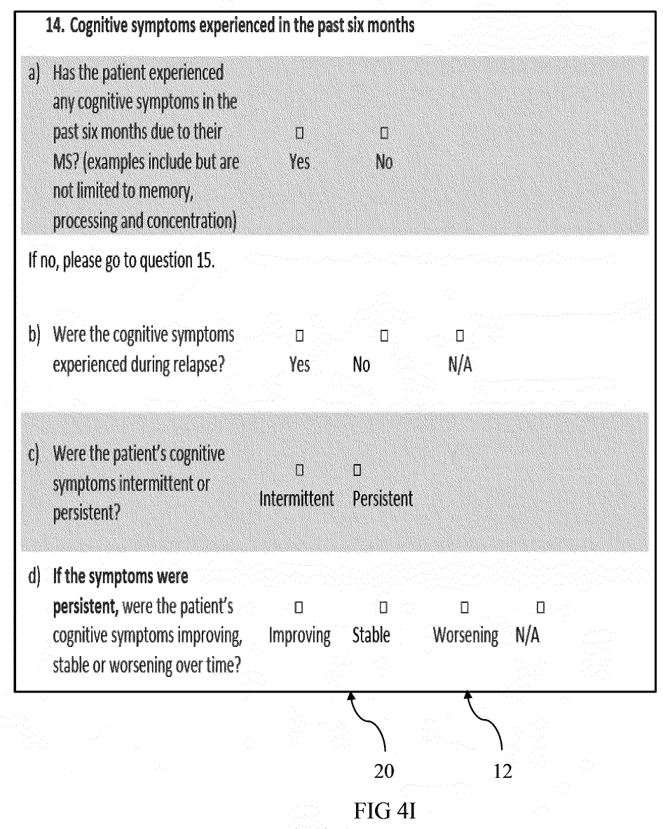

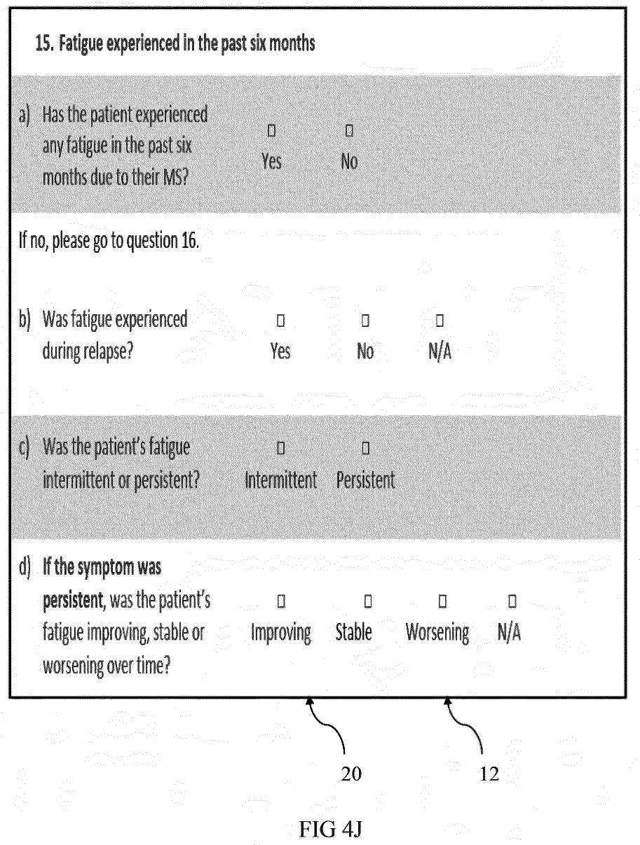

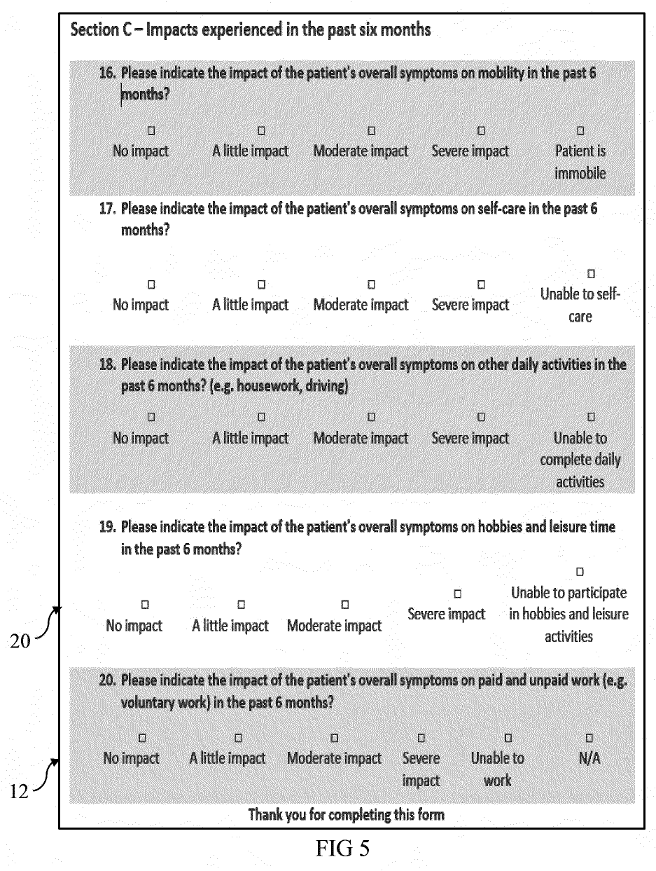

FIGS. 4A-4J depict screenshots of the GUI of the present technique illustrating items grouped into a second group along with corresponding predetermined responses for each item;

FIG. 5 depicts a screenshot of the GUI of the present technique illustrating items grouped into a third group along with corresponding predetermined responses for each item;

FIG. 6 depicts a screenshot of the GUI of the present technique illustrating EDSS query and the age query;

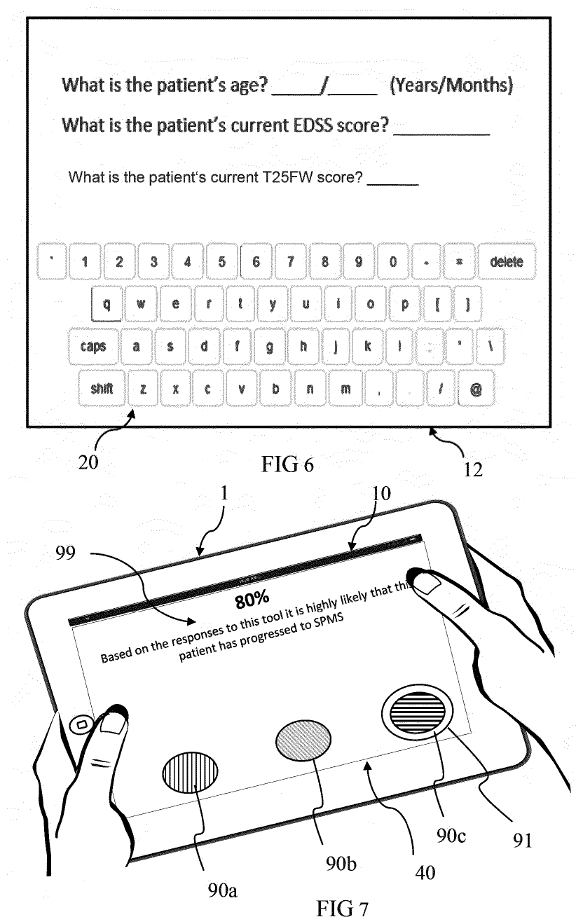

FIG. 7 schematically illustrates an embodiment of the system of the present technique indicating the state of MS determined in accordance with the present technique;

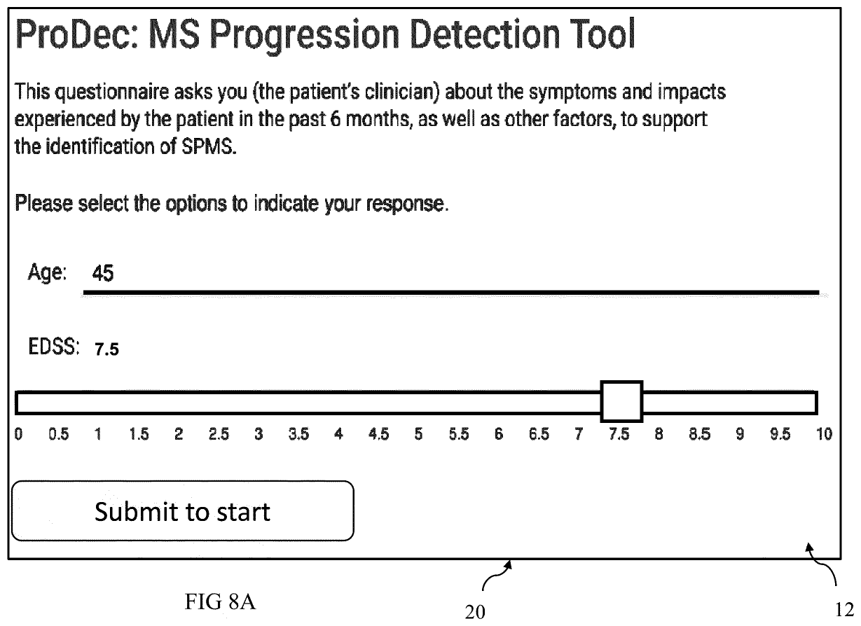

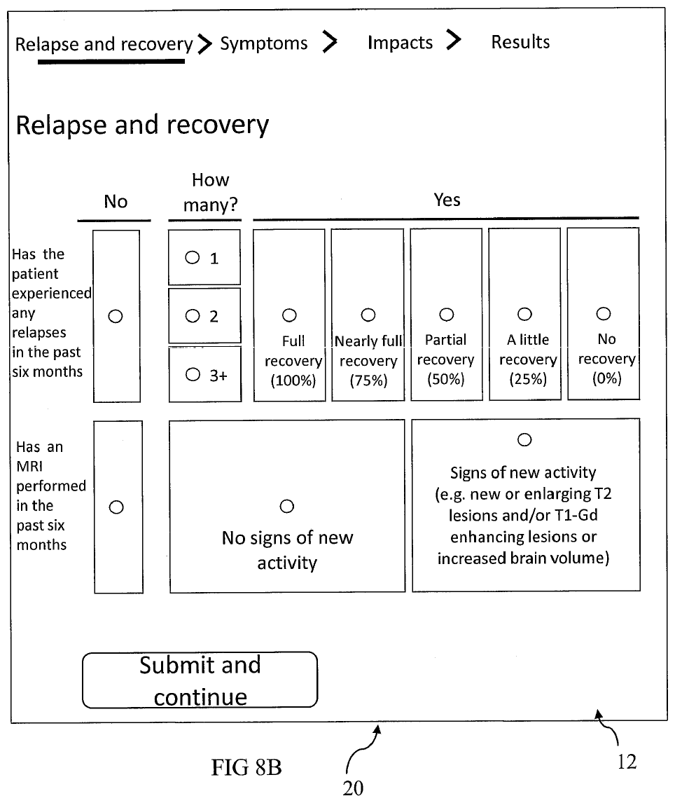

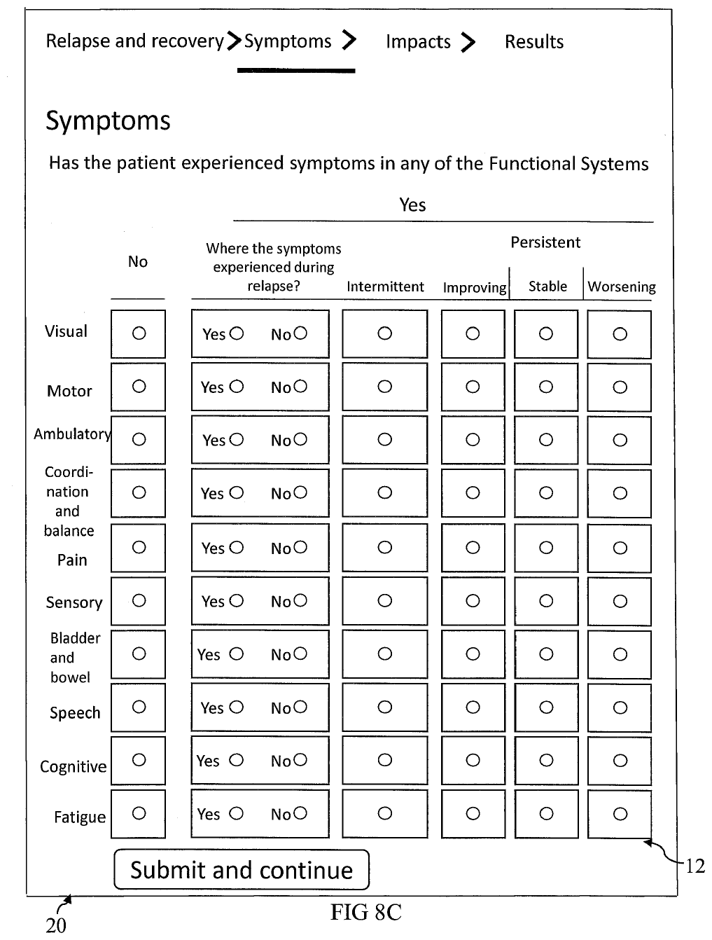

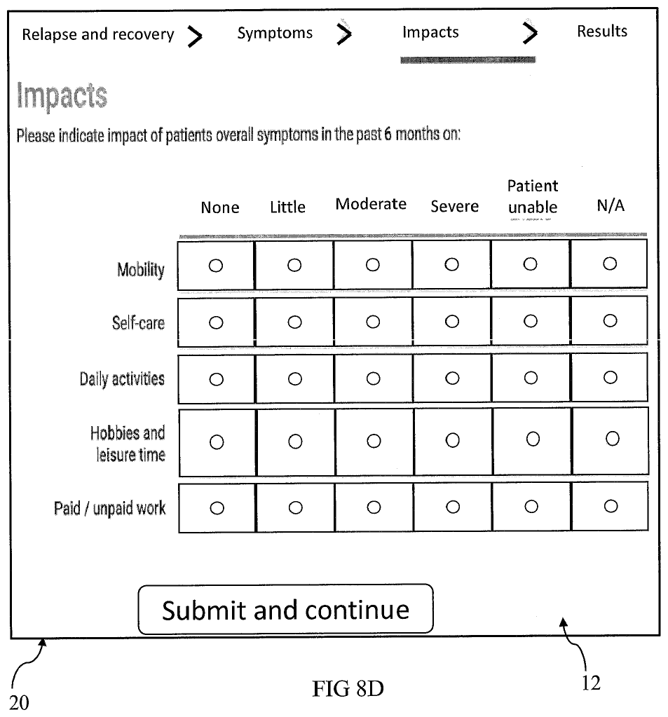

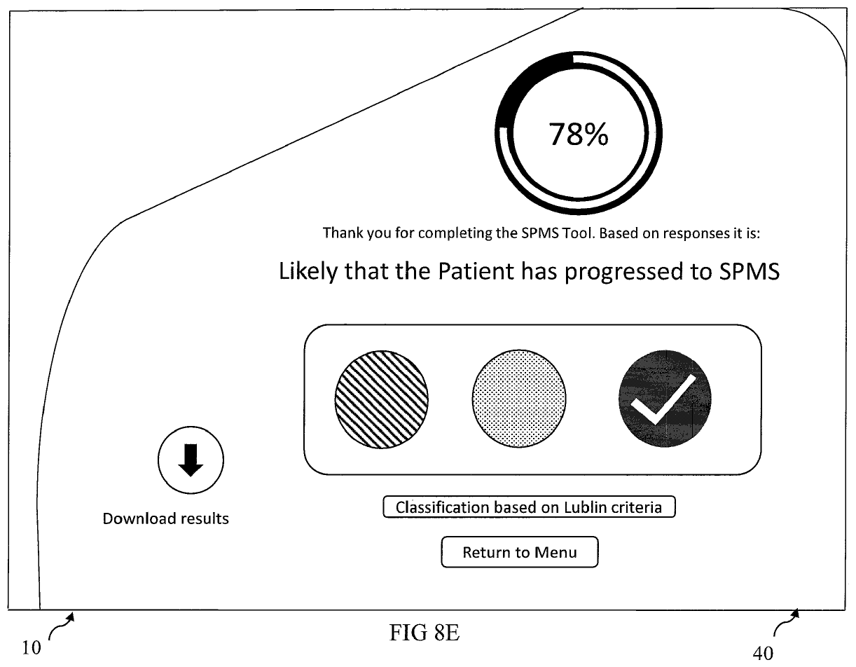

FIGS. 8A-8E depict screenshots of another embodiment of the GUI in accordance with aspects of the present technique; and

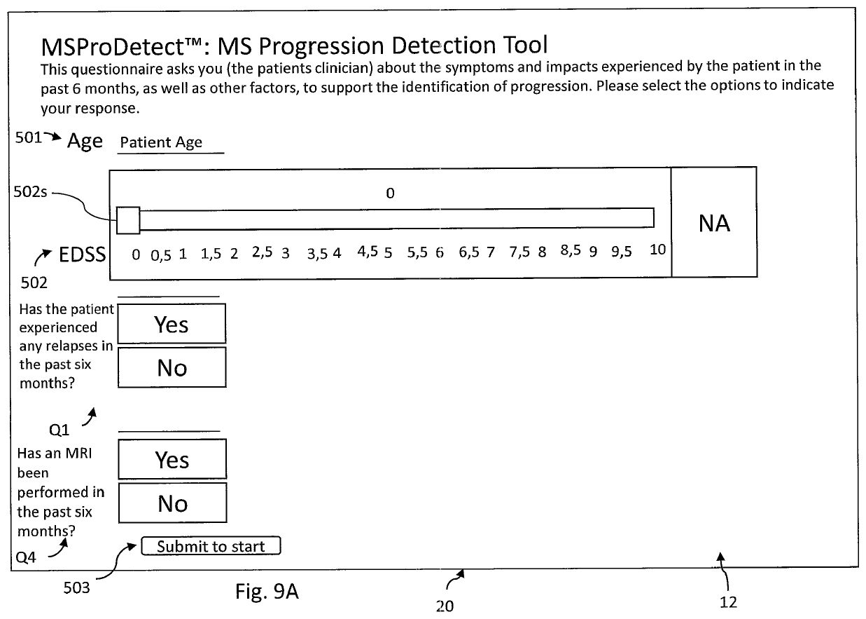

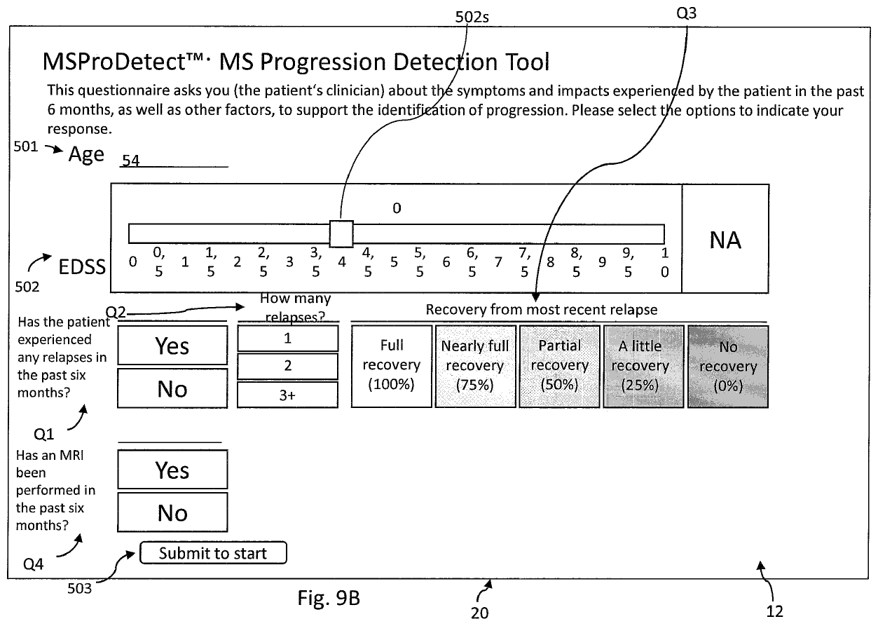

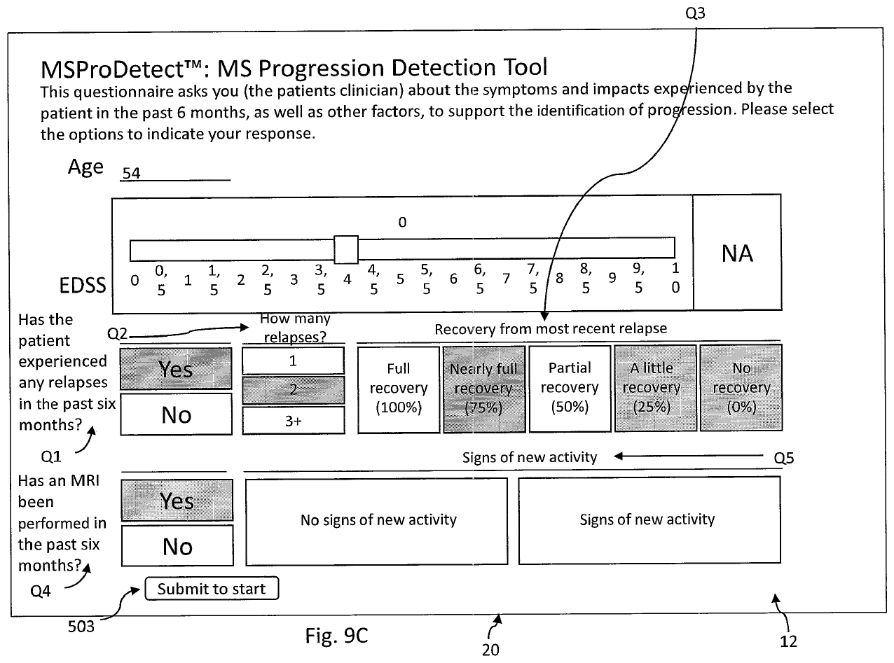

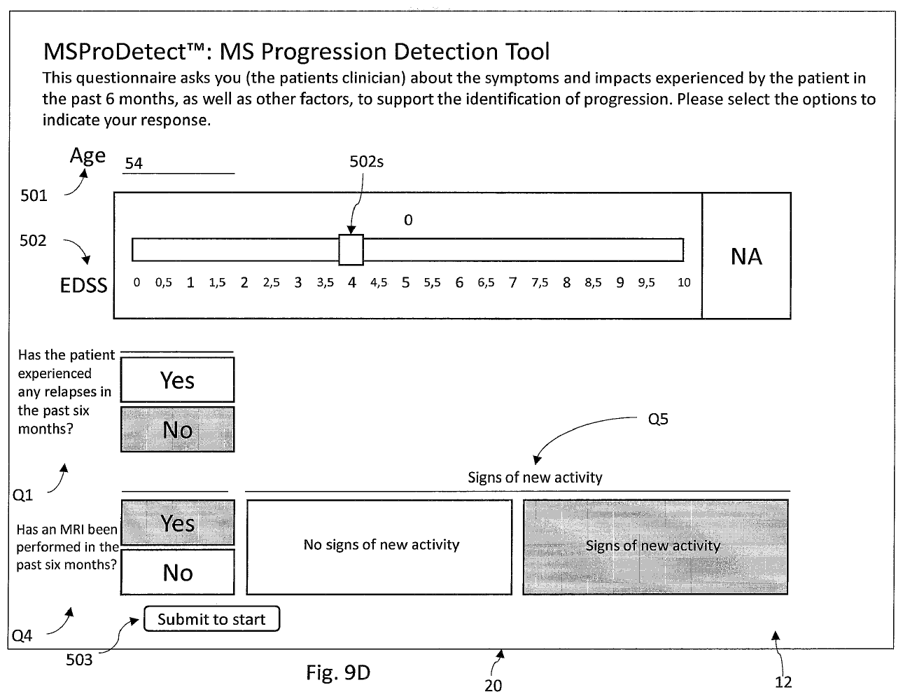

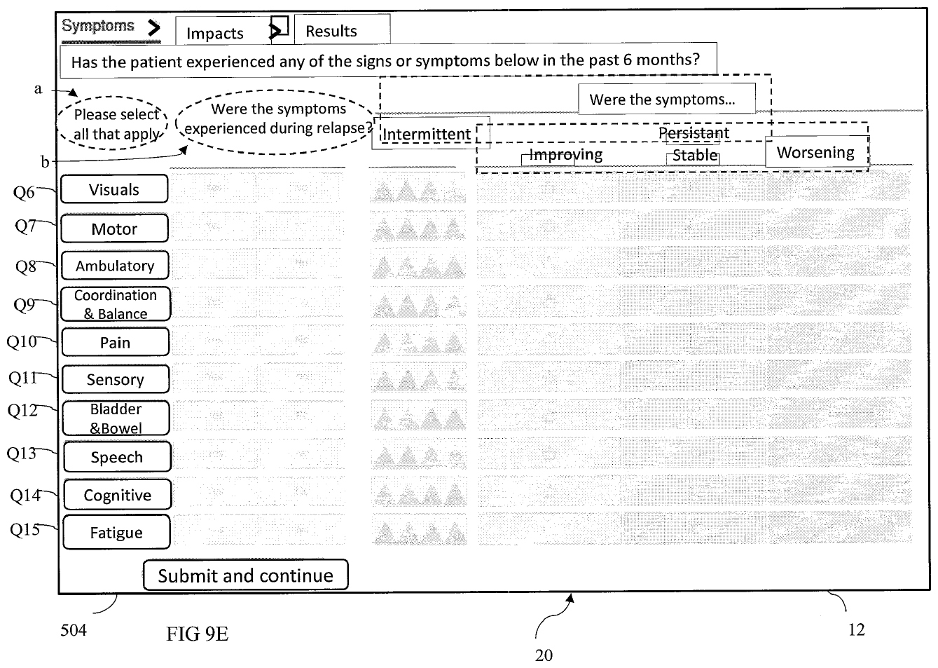

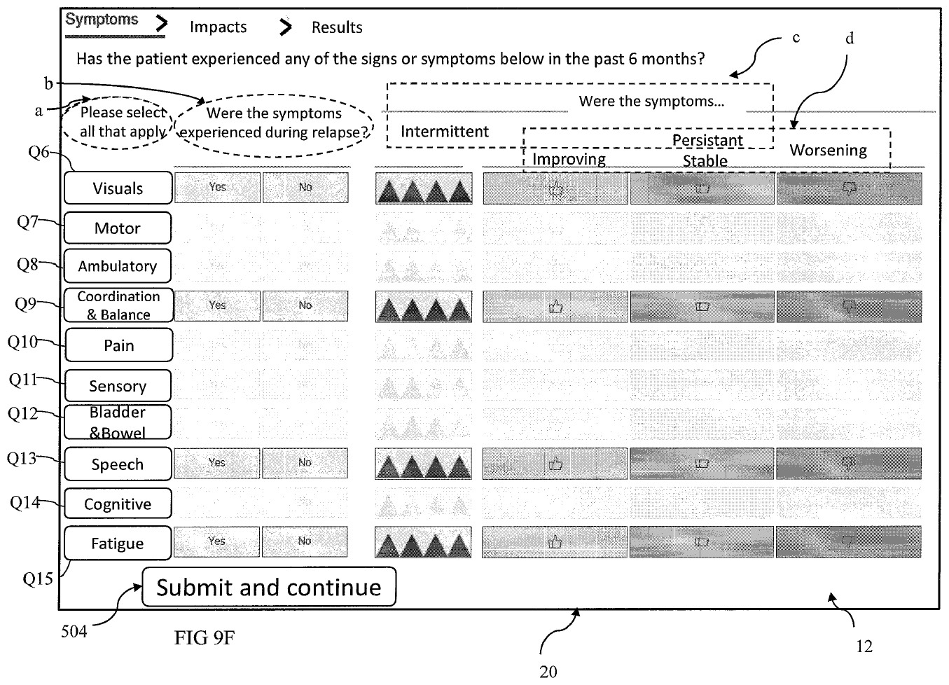

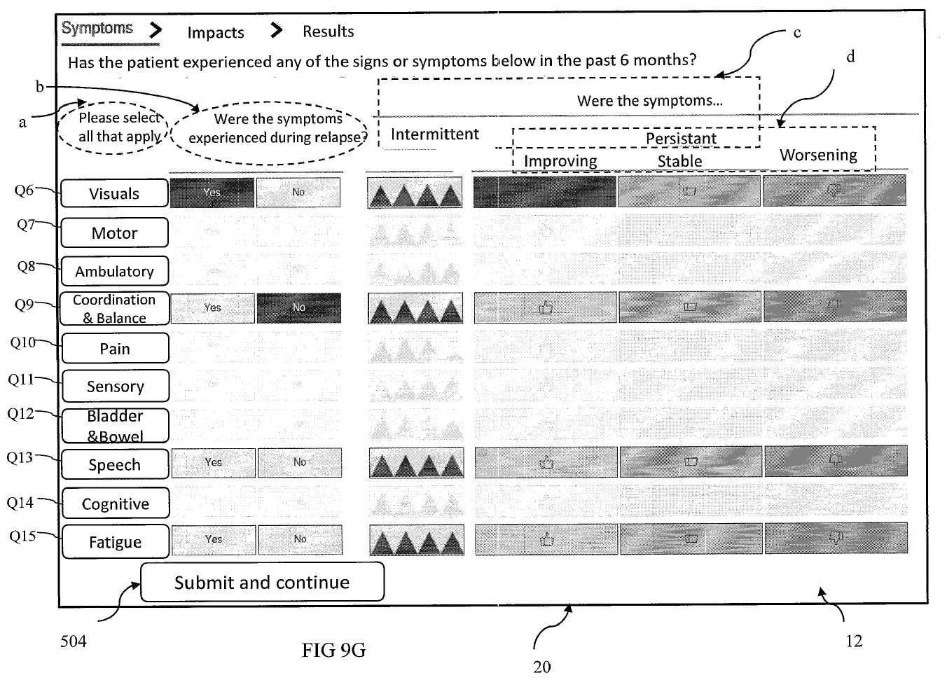

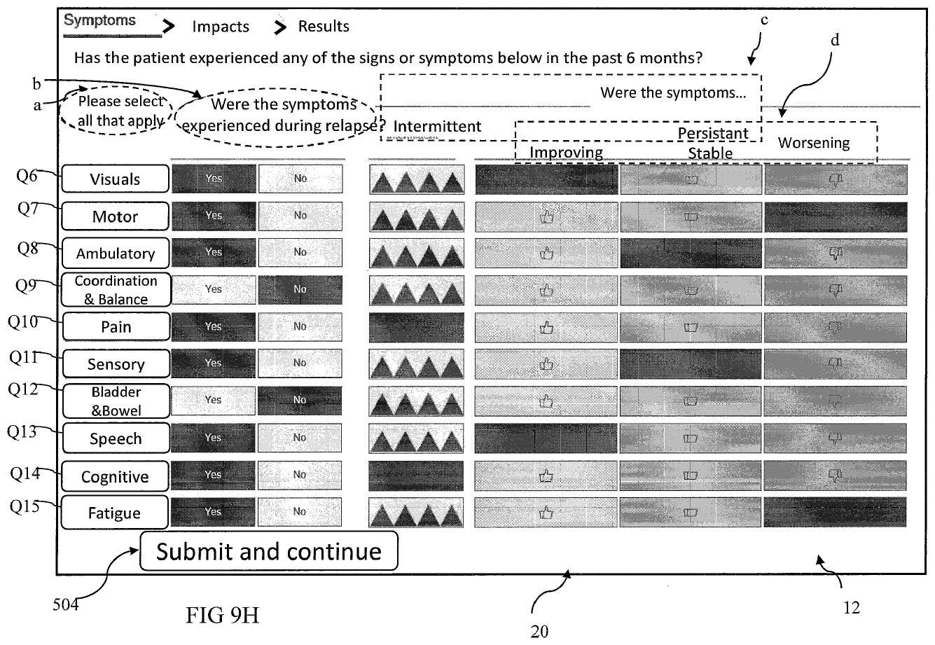

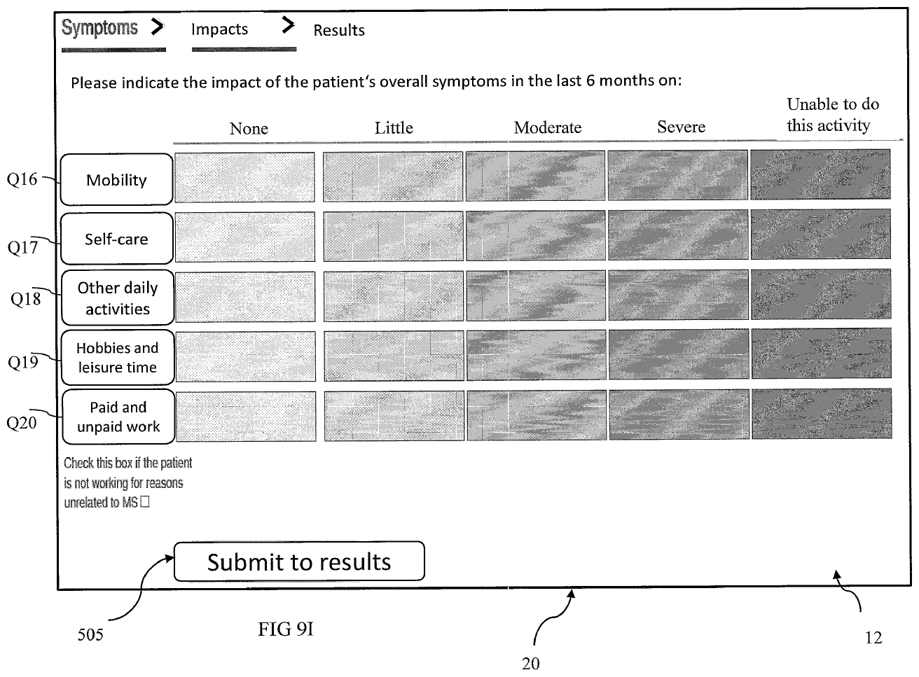

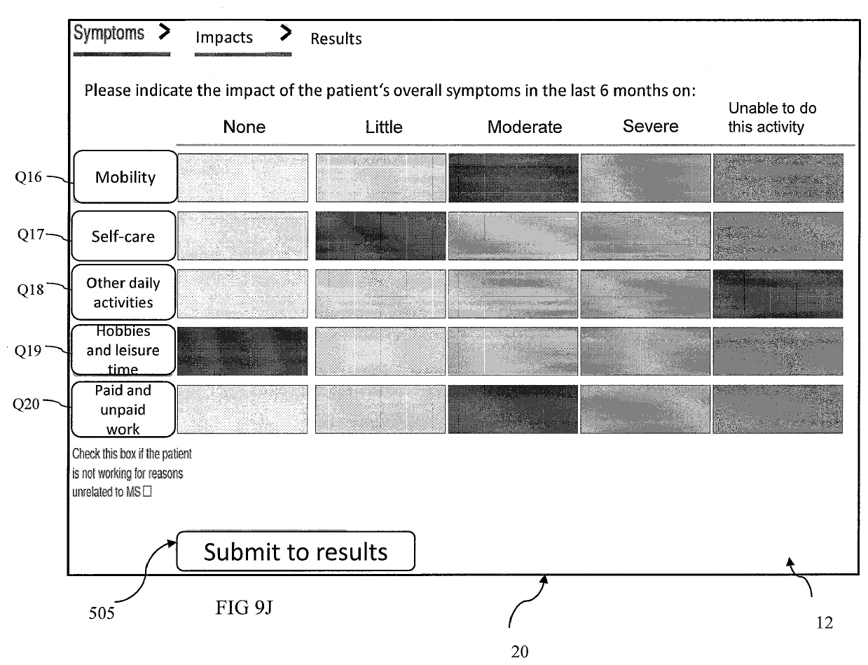

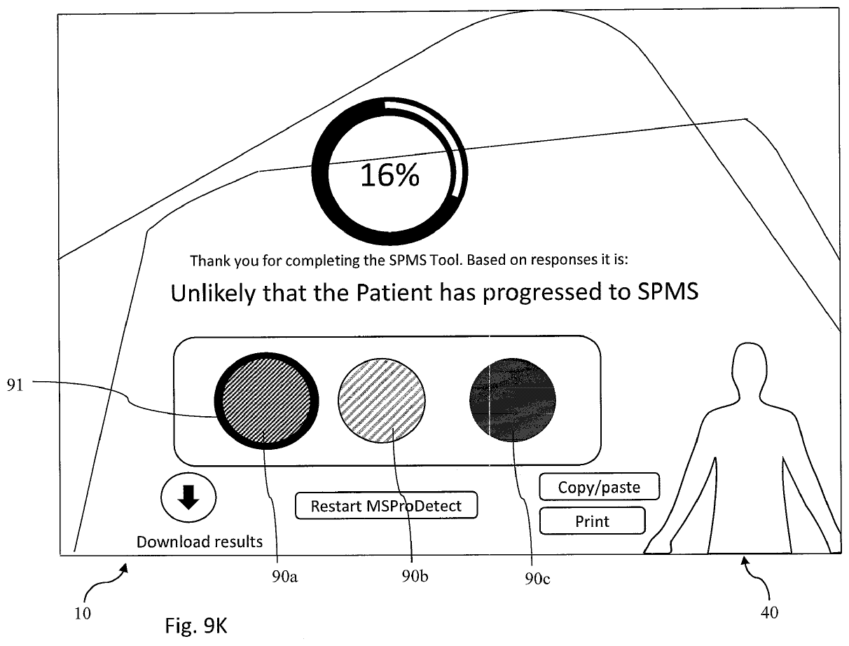

FIGS. 9A-9K depict screenshots of another embodiment of the GUI in accordance with aspects of the present technique.

Hereinafter, above-mentioned and other features of the present technique are described in detail. Various embodiments are described with reference to the drawing, wherein like reference numerals are used to refer to like elements throughout. In the following description, for purpose of explanation, numerous specific details are set forth in order to provide a thorough understanding of one or more embodiments. It may be noted that the illustrated embodiments are intended to explain, and not to limit the invention. It may be evident that such embodiments may be practiced without these specific details.

The terms first, second, third and the like in the descriptions and in the claims are used for distinguishing between elements and not necessarily for describing a sequential or chronological order, unless otherwise stated.

It may be noted that in order to provide a comprehensive understanding of the present technique, the present technique has been described hereinafter for embodiments that use EDSS and/or T25FW query and correspondingly inputted EDSS and/or T25FW score, however use of the EDSS and/or T25FW query and correspondingly inputted EDSS score and/or T25FW score is optional, i.e. non-essential, for the present technique, and thus each aspect of the present technique may be implemented or practiced without using the EDSS and/or T25FW query and correspondingly inputted EDSS and/or T25FW score. The embodiments implemented without using the EDSS and/or T25FW query and consequently implemented without the correspondingly inputted EDSS and/or T25FW score may be understood from the explanation provided hereinafter by not considering the EDSS and/or the T25FW parts.

Furthermore, the present technique has been described hereinafter for embodiments in which the first state of the MS is RRMS and the second state of MS is SPMS, however it may be noted that the first state and the second states may be states of MS other than RRMS and SPMS, for example as explained hereinabove, the first state may be ‘in-transition from RRMS to SPMS’ state when the second state is SPMS state, or the second state may be ‘in-transition from RRMS to SPMS’ state when the first state is RRMS state. Alternatively, the first state may include ‘in-transition from RRMS to SPMS’ state in addition to the RRMS state; or the second state may include ‘in-transition from RRMS to SPMS’ state in addition to the SPMS state. Also, the phrase ‘determining a progression of disease’ or ‘determining a progression of MS’ and like phrases as used herein include ‘determining the state of the disease or MS, wherein the disease is either in the first state or in the second state’. Also, in other embodiments, the present technique determines whether the state of MS in the patient is a first state, a second state or a third state, wherein the first state is ‘RRMS’ state, the second state is SPMS' state and the third state is ‘in-transition from RRMS to SPMS’ state.

FIG. 1 schematically illustrates an embodiment of a system and FIG. 2 shows a flow chart depicting a method for determining a state of Multiple Sclerosis (MS) in a patient, for example determining progression of MS in the patient from RRMS to SPMS.

The system includes a display 10, user interface 20, one or more processors 30 and an output means 40. In the present technique, hereinafter only one processor has been referred to, however it may be appreciated by one skilled in the art that one or more processors may also be used to perform the functions described for the one processor in the present technique.

The system of the present technique may be implemented on any computing device, for example but not limited to, a personal computer, a desktop computer, a tablet as shown in FIG. 7, a laptop, a personal digital assistant (PDA), a smart phone, and so on and so forth. The processor 30 causes a graphical user interface (GUI) 12 to be displayed on the display 10. The GUI 12 is displayed or output to a user. The GUI 12 includes fields for displaying patient data queries as shown in FIGS. 3 to 5 and age query as shown in FIG. 6, and optionally a field for EDSS query and/or T25FW query as also shown in FIG. 6. FIGS. 8A-8E depict screenshots of another embodiment of the GUI 12 in accordance with aspects of the present technique.

The GUI 12 having the patient data queries, EDSS query and age query is displayed to the user so that the user can read or observe the queries for providing applicable responses to the queries. As shown in FIG. 2, the method of the present technique is initiated with a step 110 wherein the GUI 12 is caused, by the processor 40, to be output on the display 10.

The patient data queries include a plurality of items. An individual patient data query is referred to as items. The items of the plurality are grouped into a first group, a second group and a third group. The first group includes at least one item relating to relapse and recovery of the patient in a first predetermined period, the second group comprises at least one item relating to symptoms experienced by the patient in a second predetermined period and the third group comprises at least one item relating to impacts experienced by the patient in a third predetermined period. Each item of the plurality of items has an assigned predetermined weight and comprises a plurality of corresponding predetermined responses. Each response of the corresponding predetermined responses has an assigned predetermined score and is indicative of a distinct information pertaining to the corresponding item.

The ‘weights’ of different items are numerical values that represent importance of a given item vis-à-vis importance of the other items in indicating a particular state of the MS, for example a numerical value that reflects the influence of that item in deciding if the MS is in a first state or in a second state, for example wherein the first state is RRMS and the second state is SPMS, the ‘weights’ of different items are numerical values that represent importance of a given item vis-à-vis importance of the other items in indicating SPMS state, for example a numerical value that reflects the influence of that item in deciding if the disease has progressed into SPMS from RRMS. In other words, when the first state is RRMS and the second state is SPMS, For example one of the numerals 1, 2 and 3 may be assigned to a given item, such as if there are a total of five items (N1, N2, N3, N4, and N5—any 5 items selected from Q1 to Q20, e.g. one item—N1—from Q1 to Q5, two items—N2 and N3—from Q6 to Q15 and two items from Q16-Q20, as shown in Tables 1, 2, and 3)—each of N1 and N3 may be assigned a weight of ‘1’, whereas N2 may be assigned a weight of ‘2’ and each of N4 and N5 may be assigned a weight of ‘3’ to reflect that each of the items N4 and N5 are more important than each of the items N1 to N3 in indicating SPMS state, whereas the item N2 is more important than each of N1 and N3 in indicating SPMS state, that N1 and N3 items are of equal or substantially equal importance in indicating SPMS state and also that the N4 and N5 items are of equal or substantially equal importance in indicating SPMS state. Simply put, when using a weight range having numerical values ‘1’, ‘2’ and ‘3’—‘3’ is assigned to each of the items that are most important, ‘2’ is assigned to each of the items that are moderately important and ‘1’ is assigned to each of the items that are least important for indicating SPMS state. It may be noted however, that the usage of weight range having numerical values ‘1’, ‘2’ and ‘3’ is for exemplary purposes only and other numerical values may be used as weights for the items in the present technique such as weights having numerical values ‘1’, ‘2’, ‘3’, ‘4’ and ‘5’, weights having numerical values ‘1’, ‘1.5″2’, ‘2.25’, 2.75’ and ‘3’, and so on and so forth. In other words, the different weights in the present technique may be represented by one or more integers, one or more fractions and a combination thereof. The weights assigned to different items may be empirically determined. The weights may be determined as a result of patient group studies and/or physician group studies and may include clinical studies.

As stated hereinabove, each item has a plurality of corresponding predetermined responses. For example, a given item may have two or three or four or five or six or seven or eight, and so on and so forth, responses. The phrase ‘score’ as used in the present technique includes numerical value that represent importance of a given response vis-à-vis importance of the other responses for indicating the effect of that item in determining a particular state of the MS, i.e. either a first state or a second state, for example wherein the first state is RRMS and the second state is SPMS. The phrase ‘score’ as used in the present technique may also include a numerical value that represent importance of a given response vis-à-vis importance of the other responses for indicating the effect of that item in determining progression from RRMS into SPMS state, for example a numerical value that reflects the influence of that item in further deciding if the disease has progressed into SPMS from RRMS. For example one of the numerals 0, 1, 2, and 3 may be assigned to a given response, such as if there are a total of five responses for a given item (say 1^{st} to 5^{th} response corresponding to a given item)—each of P^{t} response and 2^{nd} response may be assigned a score of ‘0’, 3^{rd} response may be assigned a score of ‘1’, 4^{th} response may be assigned a score of ‘2’ and 5^{th} response may be assigned a score of ‘3’ to reflect that the order of importance of that item in indicating SPMS progression is −5^{th} response (most important or most crucial) then 4^{th} response then 3^{rd} response and then each of the P^{t} and 2^{nd} responses. Simply put, when using score range having numerical values ‘0’, ‘1″2’ and ‘3’—‘3’ is assigned to each of the responses that are most important, ‘2’ is assigned to each of the responses that are moderately important, ‘1’ is assigned to each of the responses that are less important and ‘0’ is assigned to each of the responses that are least important for indicating effect of that item (to which the responses correspond to) in SPMS state. It may be noted however, that the usage of score range having numerical values ‘0’, ‘1’, ‘2’ and ‘3’ is for exemplary purposes only and other numerical values may be used as scores for the responses in the present technique such as score ranges having numerical values ‘1’, ‘2’, ‘3’, ‘4’ and ‘5’, numerical values ‘1’, ‘1.5″2’, ‘2.25’, 2.75’ and ‘3’, and so on and so forth. In other words, the different scores in the present technique may be represented by one or more integers, one or more fractions and a combination thereof. Furthermore, different score ranges may be used for responses of different items—for example, in the same method, a first item having two responses may have scores ‘0’ and ‘2’, a second item having three responses may have scores ‘0’, ‘0’, ‘2’, a third item having three responses may have scores ‘0’, ‘1’, ‘2’ and a fourth item having four responses may have scores ‘0’, ‘0’, ‘2’, ‘1’, and so on and so forth. The scores assigned to different responses for a given item may be empirically determined. The scores are determined as a result of patient group studies and/or physician group studies and may include clinical studies.

In the present technique, the ‘weights’ and ‘scores’ may be determined based on: (a) multiple logistic regression conducted on observational study variables (i.e. the items as referred to in the present disclosure) to identify how strongly each variable contributed to determination of SPMS state, (b) interviews conducted with patients and physicians to obtain qualitative insight into importance of each variable (i.e. the items as referred to in the present disclosure) in determination of SPMS state, and (c) physician ranking and weighing exercise to elicit a ranking position and individual weight contribution for each variable and to explore the level of concordance between physicians. An example of the methodology of assigning weights is provided hereinbelow in section titled ‘Methodology’.

FIG. 3, FIGS. 4A-4J and FIG. 5, respectively, show screenshots of the GUI 12 illustrating items grouped into the first group along with corresponding predetermined responses for each item of the first group, items grouped into the second group along with corresponding predetermined responses for each item of the second group and items grouped into the third group along with corresponding predetermined responses for each item of the third group. In the example of FIG. 3, FIGS. 4A-4J and FIG. 5 the predetermined period is six months (6 months) previous to and starting from a date when the present technique is being performed i.e. when a user such as a physician or healthcare provider is using the present technique to obtain a determination of whether the MS is in first state or the MS is in second state. It may be noted, that 6 months is used for exemplary purpose only, and the predetermined period may have a different duration such as for example between 3 months and 12 months e.g. 4 months, 5 months, 8 months, and so on and so forth. It may also be noted that the starting date for calculating the predetermined period may be different from the date when the present technique is being performed, for example a date when last Magnetic resonance imaging (MRI) of the patient was performed or a date when the last EDSS assessment of the patient was performed. FIG. 3, FIGS. 4A-4J and FIG. 5, respectively, show the first group, the second group and the third group as ‘Section A’, ‘Section B’ and ‘Section C’. The computer-executable instructions that provide the GUI and the groups, the items of the groups and the responses to each of the items is stored in a memory of the system and thereafter loaded and executed by the processor. The display 10 may include an icon (not shown) which when selected by a user, desirous of performing the present technique, may cause the loading and execution of the computer-executable instructions leading to the displaying of the GUI 12 on the display 10. FIG. 3, FIG. 4A and FIG. 5 each depict the GUI 12 in which an indication 121 of the group, i.e. whether the first group or the second group or the third group, being displayed is provided, also the items 122 belonging to the group and the responses 123 for each of the items is displayed. FIGS. 4A to 4J provide screenshots in series representing the second group.

The user interface 20 is for inputting of responses to the patient data queries, for inputting of an EDSS score of the patient as a response to the EDSS query and for inputting of an age of the patient as a response to the age query. The user interface 20 may be, but not limited to, a touch-user interface operable by touch of the user, for example with user's finger or a stylus, or may be another type of user interface such as computer keyboard, a computer mouse, a voice-user interface. In one embodiment of the system of the present technique, the display 10 is a touch-screen display and hence also functions as the user interface 20.

The method, after the step 110, includes a step 120 of inputting of responses to the patient data queries by selecting at least one response from the plurality of corresponding predetermined responses for each of the plurality of items displayed in the GUI 12. The method further includes, after the step 110, a step 150 of inputting of an EDSS score of the patient as a response to the EDSS query in the GUI 12. Furthermore, the method includes, after the step 110, a step 170 of inputting of an age of the patient as a response to the age query in the GUI 12. The steps 120, 150 and 170 are independent of each other and may be performed in any order. For example, but not limited to, a user may first perform the step 120 then 150 and thereafter 170, or alternatively first perform the step 150 then 170 and thereafter 120 or first perform the step 170 then 150 and thereafter 120, and so on and so forth. In other words, the GUI 12 may first display FIG. 3 then FIGS. 4A to 4J then FIG. 5, or alternatively the GUI 12 may first display FIGS. 4A to 4J then FIG. 5 and then FIG. 3, or alternatively, the GUI 12 may first display FIG. 5 then FIGS. 4A to 4J then FIG. 3, so on and so forth.

The user inputs the response by selecting, via the user interface 20, at least one response from the plurality of corresponding predetermined responses for each of the plurality of items displayed in the GUI 12, as shown in FIGS. 3 to 5. Each response of the corresponding predetermined responses is indicative of a distinct information pertaining to the corresponding item, for example as shown in FIG. 3 for the item—‘How many relapses has the patient experienced in the past 6 months’ the different responses show ‘1’, ‘2’, ‘3 or more’. The user i.e. for example a physician or a person administering the present technique selects the response that is applicable to the patient being assessed. For example, as shown in FIG. 3, a ‘X’ symbol is displayed when the user selects a response ‘2’ for the item number 2 i.e. the item—‘How many relapses has the patient experienced in the past 6 months’. However, if the patient had experienced 4 relapses in the past 6 months, then the user would select the applicable response from the corresponding predetermined responses i.e. the response—‘3 or more’. Alternatively, if the patient had experienced 1 relapse in the past 6 months, then the user would select the applicable response from the corresponding predetermined responses i.e. the response—‘1’.

In the present technique, applicable to the system shown in FIG. 1 and method shown in FIG. 2 of the present technique, the items of the first group are selected from: an item indicating if the patient has experienced any relapses (Q1), an item indicating a number of relapses the patient has experienced (Q2), an item indicating an extent of recovery of the patient from a last relapse (Q3), an item indicating if a Magnetic Resonance Imaging, MRI, has been performed on the patient (Q4), and an item indicating if the performed MRI showed new signs of activity related to MS (Q5)—as shown herein in Table 1. One or more of the aforementioned items, Q1 to Q5, may be selected from the items enlisted in Table 1 for example, in one embodiment the first group includes one of Q1 to Q5, whereas in another embodiment the first group includes two of Q1 to Q5, whereas in yet another embodiment the first group includes three of Q1 to Q5, whereas in yet another embodiment the first group includes four of Q1 to Q5, and in yet another embodiment the first group includes all of Q1 to Q5.

TABLE 1
First Group-items and corresponding predetermined responses
(N/A = Not applicable)

| | | Corresponding predetermined |
|---|---|---|
| | | responses to select from |
| Items | Item details | (responses within quotes) |
| Q1 | Has the patient experienced any | ‘Yes’ or ‘No’ |
| | relapses in the past 6 months? | |
| Q2 | How many relapses has the patient | ‘1’ or ‘2’ or ‘3 or more’ |
| | experienced in the past 6 months? | |
| Q3 | Please rate the patient's recovery | ‘Full recovery (100%)’ or |
| | from their most recent relapse | ‘Nearly full recovery (75%)’ |
| | | or ‘Partial recovery (50%)’ |
| | | or ‘Little recovery (25%)’ |
| | | or ‘No recovery (0%)’ |
| Q4 | Has an MRI been performed in | ‘Yes’ or ‘No’ |
| | the past six months? | |
| Q5 | If ‘Yes’ to Q4, does the MRI | ‘Yes’ or ‘No’ |
| | indicate signs of new activity | |
| | (e.g. new or enlarging | |
| | gadolinium enhanced T1 | |
| | weighted lesions or increased | |
| | brain volume)? | |

In the present technique, applicable to the system shown in FIG. 1 and method shown in FIG. 2 of the present technique, the items of the second group are selected from: an item indicating visual symptoms related to MS (Q6), an item indicating motor symptoms related to MS (Q7), an item indicating ambulatory symptoms related to MS (Q8), an item indicating coordination and balance symptoms related to MS (Q9), an item indicating pain experienced due to MS (Q10), an item indicating sensory symptoms related to MS (Q11), an item indicating bladder and bowel symptoms related to MS (Q12), an item indicating speech symptoms related to MS (Q13), an item indicating cognitive symptoms related to MS (Q14), and an item indicating fatigue symptoms related to MS (Q15)—as shown herein in Table 2. One or more of the aforementioned items, Q6 to Q15 may be selected from the items enlisted in Table 2 for example, in one embodiment the second group includes one of Q6 to Q15, in another embodiment the second group includes two of Q6 to Q15, in yet another embodiment the second group includes three of Q6 to Q15, in yet another embodiment the second group includes four of Q6 to Q15, in another embodiment the second group includes five of Q6 to Q15, in yet another embodiment the second group includes six of Q6 to Q15, in yet another embodiment the second group includes seven of Q6 to Q15, in another embodiment the second group includes eight of Q6 to Q15, in yet another embodiment the second group includes nine of Q6 to Q15, and in yet another embodiment the second group includes all of Q6 to Q15.

TABLE 2
Second Group-items and corresponding predetermined responses
(N/A = Not applicable)

| | | | Corresponding |
|---|---|---|---|
| | | | predetermined responses |
| Items-Item | Sub- | | to select from |
| details | Items | Sub-item details | (responses within quotes) |
| Q6-Visual | Q6a | Has the patient experienced any visual | ‘Yes’ or ‘No’ |
| symptoms | | symptoms in the past six months due to | |
| experienced | | their MS? (examples include but are not | |
| in the past six | | limited to double vision, optic neuritis | |
| months | | and decreased acuity) | |
| | Q6b | Were the visual symptoms experienced | ‘Yes’ or ‘No’ or ‘N/A’ |
| | | during relapse? | |
| | Q6c | Were the patient's visual symptoms | ‘Intermittent’ or |
| | | intermittent or persistent? | ‘Persistent’ |
| | Q6d | If the symptoms were persistent, were | ‘Improving’ or ‘Stable’ or |
| | | the patient's visual symptoms | ‘Worsening’ or ‘N/A’ |
| | | improving, stable or worsening over | |
| | | time? | |
| Q7-Motor | Q7a | Has the patient experienced any motor | ‘Yes’ or ‘No’ |
| symptoms | | symptoms in the past six months due to | |
| experienced | | their MS? (examples include but are not | |
| in the past six | | limited to muscle weakness or paralysis) | |
| months | | | |
| | Q7b | Were the motor symptoms experienced | ‘Yes’ or No, or ‘N/A’ |
| | | during relapse? | |
| | Q7c | Were the patient's motor symptoms | ‘Intermittent’ or |
| | | intermittent or persistent? | ‘Persistent’ |
| | Q7d | If the symptoms were persistent, were | ‘Improving’ or 'Stable' or |
| | | the patient's motor symptoms | ‘Worsening’ or ‘N/A’ |
| | | improving, stable or worsening over | |
| | | time? | |
| Q8-Ambulatory | Q8a | Has the patient experienced any | ‘Yes’ or ‘No’ |
| symptoms | | ambulatory symptoms in the past six | |
| experienced | | months due to their MS? (examples | |
| in the past six | | include but are not limited to gait and | |
| months | | mobility) | |
| | Q8b | Were the ambulatory symptoms | ‘Yes’ or ‘No’ or ‘N/A’ |
| | | experienced during relapse? | |
| | Q8c | Were the patient's ambulatory | ‘Intermittent’ or |
| | | symptoms intermittent or persistent? | ‘Persistent’ |
| | Q8d | If the symptoms were persistent, were | ‘Improving’ or 'Stable' or |
| | | the patient's ambulatory symptoms | ‘Worsening’ or ‘N/A’ |
| | | improving, stable or worsening over | |
| | | time? | |
| Q9-Coordination | Q9a | Has the patient experienced any | ‘Yes’ or ‘No’ |
| and balance | | coordination and balance symptoms | in |
| symptoms | | the past six months due to their MS? | |
| experienced | | (examples include but are not limited to | |
| in the past six | | ataxia, balance and fine motor skills) | |
| months | Q9b | Were the coordination and balance | ‘Yes’ or ‘No’ or ‘N/A’ |
| | | symptoms experienced during relapse? | |
| | Q9c | Were the patient's coordination and | ‘Intermittent’ or |
| | | balance symptoms intermittent or | ‘Persistent’ |
| | | persistent? | |
| | Q9d | If the symptoms were persistent, were | ‘Improving’ or 'Stable' or |
| | | the patient's coordination and balance | ‘Worsening’ or ‘N/A’ |
| | | symptoms improving, stable or | |
| | | worsening over time? | |
| Q10-pain | Q10a | Has the patient experienced any pain in | ‘Yes’ or ‘No’ |
| experienced | | the past six months due to their MS? | |
| in the past six | | (examples include but are not limited to | |
| months | | neuropathic pain or central pain) | |
| | Q10b | Was the pain experienced during | ‘Yes’ or ‘No’ or ‘N/A’ |
| | | relapse? | |
| | Q10c | Was the patient's pain intermittent or | ‘Intermittent’ or |
| | | persistent? | ‘Persistent’ |
| | Q10d | If the pain was persistent, was the | ‘Improving’ or 'Stable' or |
| | | patient's pain improving, stable or | ‘Worsening’ or ‘N/A’ |
| | | worsening over time? | |
| Q11-sensory | Q11a | Has the patient experienced any sensory | ‘Yes’ or ‘No’ |
| symptoms | | symptoms in the past six months due to | |
| experienced | | their MS? (examples include but are not | |
| in the past six | | limited to numbness and tingling) | |
| months | Q11b | Were the sensory symptoms | ‘Yes’ or ‘No’ or ‘N/A’ |
| | | experienced during relapse? | |
| | Q11c | Were the patient's sensory symptoms | ‘Intermittent’ or |
| | | intermittent or persistent? | ‘Persistent’ |
| | Q11d | If the symptoms were persistent, were | ‘Improving’ or 'Stable' or |
| | | the patient's sensory symptoms | ‘Worsening’ or ‘N/A’ |
| | | improving, stable or worsening over | |
| | | time? | |
| Q12- | Q12a | Has the patient experienced any B&B | ‘Yes’ or ‘No’ |
| Bladder and | | symptoms in the past six months due to | |
| bowel (B&B) | | their MS? | |
| symptoms | Q12b | Were the B&B symptoms experienced | ‘Yes’ or ‘No’ or ‘N/A’ |
| experienced | | during relapse? | |
| in the past six | Q12c | Were the patient's B&B symptoms | ‘Intermittent’ or |
| months | | intermittent or persistent? | ‘Persistent’ |
| | Q12d | If the symptoms were persistent, were | ‘Improving’ or 'Stable' or |
| | | the patient's B&B symptoms improving, | ‘Worsening’ or ‘N/A’ |
| | | stable or worsening over time? | |
| Q13-speech | Q13a | Has the patient experienced any speech | ‘Yes’ or ‘No’ |
| symptoms | | symptoms in the past six months due to | |
| experienced | | their MS? (examples include but are not | |
| in the past six | | limited to dysarthria and aphasia) | |
| months | Q13b | Were the speech symptoms experienced | ‘Yes’ or ‘No’ or ‘N/A’ |
| | | during relapse? | |
| | Q13c | Were the patient's speech symptoms | ‘Intermittent’ or |
| | | intermittent or persistent? | ‘Persistent’ |
| | Q13d | If the symptoms were persistent, were | ‘Improving’ or 'Stable' or |
| | | the patient's speech symptoms | ‘Worsening’ or ‘N/A’ |
| | | improving, stable or worsening over | |
| | | time? | |
| Q14-cognitive | Q14a | Has the patient experienced any | ‘Yes’ or ‘No’ |
| symptoms | | cognitive symptoms in the past six | |
| experienced | | months due to their MS? (examples | |
| in the past six | | include but are not limited to memory, | |
| months | | processing and concentration) | |
| | Q14b | Were the cognitive symptoms | ‘Yes’ or ‘No’ or ‘N/A’ |
| | | experienced during relapse? | |
| | Q14c | Were the patient's cognitive symptoms | ‘Intermittent’ or |
| | | intermittent or persistent? | ‘Persistent’ |
| | Q14d | If the symptoms were persistent, were | ‘Improving’ or 'Stable' or |
| | | the patient's cognitive symptoms | ‘Worsening’ or ‘N/A’ |
| | | improving, stable or worsening over | |
| | | time? | |
| Q15-fatigue | Q15a | Has the patient experienced any fatigue | ‘Yes’ or ‘No’ |
| experienced | | in the past six months due to their MS? | |
| in the past six | Q15b | Was the fatigue experienced during | ‘Yes’ or ‘No’ or ‘N/A’ |
| months | | relapse? | |
| | Q15c | Was the patient's fatigue intermittent or | ‘Intermittent’ or |
| | | persistent? | ‘Persistent’ |
| | Q15d | If the fatigue was persistent, was the | ‘Improving’ or 'Stable' or |
| | | patient's fatigue improving, stable or | ‘Worsening’ or ‘N/A’ |
| | | worsening over time? | |

Each of the items of the second group may further be classified into sub-items as shown in Table 2. Each of the sub-items have their corresponding predetermined responses having their predetermined scores. The score of a given item may be calculated or determined for example, but not limited to, by an averaging of the scores of the selected responses of the sub-items or by an addition of the scores of the selected responses of the sub-items, or by weighted averaging of the scores of the selected responses of the sub-items or any other suitable statistical operation. In one embodiment, the score for a given item may be calculated by averaging the scores of responses selected for the sub-items or by selecting the highest score from the scores of responses selected for the sub-items.

In the present technique, applicable to the system shown in FIG. 1 and method shown in FIG. 2 of the present technique, the items of the third group are selected from: an item indicating impact on mobility of the patient due to MS (Q16), an item indicating impact on self-care of the patient due to MS (Q17), an item indicating impact on daily activities of the patient due to MS (Q18), an item indicating impact on hobbies and leisure time of the patient due to MS (Q19), and an item indicating impact on paid and unpaid work of the patient due to MS (Q20)—as shown herein in Table 3. One or more of the aforementioned items, Q16 to Q20, may be selected form the items enlisted in Table 3 for example, in one embodiment the third group includes one of Q16 to Q20, whereas in another embodiment the third group includes two of Q16 to Q20, whereas in yet another embodiment the third group includes three of Q16 to Q20, whereas in yet another embodiment the third group includes four of Q16 to Q20, and in yet another embodiment the third group includes all of Q16 to Q20.

TABLE 3
Third Group-items and corresponding predetermined responses
(N/A = Not applicable)

| | | Corresponding responses to select |
|---|---|---|
| Items | Item details | from (responses within quotes) |
| Q16 | Please indicate the impact of the patient's | ‘No impact’ or ‘Little impact’ or |
| | overall symptoms on mobility in the past | ‘Moderate impact’ or ‘Severe |
| | 6 months? | impact’ or ‘Patient is immobile’ |
| Q17 | Please indicate the impact of the patient's | ‘No impact’ or ‘Little impact’ or |
| | overall symptoms on self-care in the past | ‘Moderate impact’ or ‘Severe |
| | 6 months? | impact’ or ‘Unable to self-care’ |
| Q18 | Please indicate the impact of the patient's | ‘No impact’ or ‘Little impact’ or |
| | overall symptoms on other daily | ‘Moderate impact’ or ‘Severe |
| | activities in the past 6 months? | impact’ or ‘Unable to complete |
| | (e.g. housework, driving) | daily activities’ |
| Q19 | Please indicate the impact of the patient's | ‘No impact’ or ‘Little impact’ or |
| | overall symptoms on hobbies and leisure | ‘Moderate impact’ or ‘Severe |
| | time in the past 6 months? | impact’ or ‘Unable to participate |
| | | in hobbies and leisure activities’ |
| Q20 | Please indicate the impact of the patient's | ‘No impact’ or ‘Little impact’ or |
| | overall symptoms on paid and unpaid | ‘Moderate impact’ or ‘Severe |
| | work (e.g. voluntary work) in the past 6 | impact’ or ‘Unable to work’ |
| | months? | |

In the present technique, the GUI 12 may therefore include—one or more of items selected, as explained hereinabove, from Q1 to Q5 as shown in Table 1, and one or more of items selected, as explained hereinabove, from Q6 to Q15 as shown in Table 2, and one or more of items selected, as explained hereinabove, from Q16 to Q20, as shown in Table 3 to form the first group, the second group and the third group, respectively.

Furthermore, in an embodiment of the present technique, as depicted by the example of FIG. 3, FIGS. 4A-4J and FIG. 5 the first group, the second group and the third group may include, respectively, all the items from Q1 to Q5 of Table 1, all the items and corresponding sub-items from Q6 to Q15 of Table 2 and all the items from Q16 to Q20 of Table 3.

FIG. 6 depicts a screenshot of the GUI of the present technique illustrating EDSS query and the age query. The user can fill in the responses to the EDSS query and age query using a virtual keyboard such as depicted in FIG. 6. Alternatively, the GUI may present a drop-down menu from which the user selects the applicable response i.e. the age of the patient and the EDSS score of the patient.

The responses to the patient data queries, the EDSS query and the age query inputted by the user are received by the processor 30 shown in FIG. 1.

The processor 30 determines an item score for each of the plurality of items based on the weight of the item and the score of the selected response for the item. In the method of the present technique, after the step 120, is a step 130 of determining, by the processor 30, the item score for each of the plurality of items based on the weight of the item and the score of the selected response for the item. In one embodiment of the present technique, the one or more processors is configured to determine the item score by multiplying the weight of the item and the score of the selected response for the item.

Thereafter, the processor 30 determine a first group score, a second group score and a third group score based on the item scores of the items of the first group, the second group and the third group, respectively. The method of the present technique, after step 130, includes a step 140 of determining, by the processor 30, the first group score, the second group score and the third group score based on the item scores of the items of the first group, the second group and the third group, respectively.

The weights of the items, or of the corresponding sub-items where applicable, and scores of the responses to the items or scores of the responses to the sub-items, where applicable may be stored in a look-up table or any other suitable data storage format in the system, for example in a memory of the system or may be stored remotely such as in cloud or a server and may be accessed, via wired or wireless connection, by the system of the present technique by using a network such as internet, intranet, local area network, and so on and so forth. Continuing the example of the embodiment of FIG. 3, FIGS. 4A-4J and FIG. 5, Tables 4, 5 and 6 depict items, item weights, corresponding predetermined responses and response scores for the first group, the second group and the third group, respectively. In one embodiment of the system, the one or more processors 30 is configured to determine the first group score, the second group score and the third group score by adding the item scores of the items comprised in the first group, the second group and the third group, respectively. As stated hereinabove, when each of the items of the second group are further classified into sub-items as shown in Table 2, the score of a given item of the second group may be calculated or determined for example, but not limited to, by an averaging of the scores of the selected responses of the sub-items or by an addition of the scores of the selected responses of the sub-items, or any other suitable statistical operation or for example by weighted averaging of the scores of the selected responses of the sub-items.

TABLE 4
First Group-items, item weights, corresponding predetermined
responses and response scores

| | Item | Response | |
|---|---|---|---|
| | weight | Score | Corresponding predetermined |
| Items | (wA) | (rA) | responses and scores |
| Q1 | w1 | r1 | ‘Yes’ = r11; ‘No’ = r12 |
| Q2 | w2 | r2 | ‘1’ = r21; ‘2’ = r22; ‘3 or more’ = r23 |
| Q3 | w3 | r3 | ‘Full recovery (100%)’ = r31; |
| | | | ‘Nearly full recovery (75%)’ = r32; |
| | | | ‘Partial recovery (50%)’ = r33; |
| | | | ‘Little recovery (25%)’ = r34; |
| | | | ‘No recovery (0%)' = r35 |
| Q4 | w4 | r4 | ‘Yes’ = r41; ‘No’ = r42 |
| Q5 | w5 | r5 | ‘Yes’ = r51; ‘No’ = r52 |

The weights w1 to w5 of the items Q1 to Q5 may range from numerical values 1 to 4. In an embodiment of the present technique, the items Q1 and Q5 are more important than the items Q2 and Q3 and therefore each of the items Q1 and Q5 are assigned higher weights than each of the items Q2 and Q3. The scores, r11, r12, r21, r22, r23, r31, r32, r33, r34, r35, r41, r42, r51 and r52 as enlisted in Table 4 may range from numerical values 0 to 4. For example, when both Q4 and Q5 are included in the first group, r41 and r42 have scores assigned as zero.

The first group score may be calculated as:

$$First Group score = {\sum }_{A = 1}^{5} wA * rA$$

wherein, A represents the item number between first and the fifth item from Table 1 or Table 4 i.e. Q1 and Q5, and wA represents the weight corresponding to that item, and rA represents the score of the response corresponding to that item.

In the above equation, rA is substituted with the score of the selected response for that item, for example when A=1, i.e. when the item is Q1, if the selected response is ‘Yes’ then rA which is r1 is substituted by r11 whereas if the selected response is ‘No’ then rA which is r1 is substituted by r12. Similarly, when A=3, i.e. when the item is Q3, if the selected response is ‘Full recovery (100%)’ then rA which is r3 is substituted by r31; if the selected response is ‘Nearly full recovery (75%)’ then rA which is r3 is substituted by r32; if the selected response is ‘Partial recovery (50%)’ then rA which is r3 is substituted by r33; if the selected response is ‘Little recovery (25%)’ then rA which is r3 is substituted by r34; if the selected response is ‘No recovery (0%)’ then rA which is r3 is substituted by r35. Similarly, rA is also substituted with the score of the selected response for that items Q2 to Q5 when included in the first group, as explained hereinabove for rA of Q1.

TABLE 5
Second Group—items, item weights, corresponding
predetermined responses and response scores

| | | Score | | | |
|---|---|---|---|---|---|
| | Weight | of | | | |
| | of the | the | | Sub- | Corresponding |
| | item | item | Sub- | item | predetermined responses |
| Items | (wB) | (wB) | Items | weights | to sub-items and their scores |
| Q6 | w6 | r6 | Q6a | w6a | ‘Yes’ = r6a1; ‘No’ = r6a2 |
| | | | Q6b | w6b | ‘Yes’ = r6b1; ‘No’ = r6b2; |
| | | | | | ‘N/A’ = r6b3 |
| | | | Q6c | w6c | ‘Intermittent’ = r6c1 ; |
| | | | | | ‘Persistent’ = r6c2 |
| | | | Q6d | w6d | ‘Improving’ = r6d1; |
| | | | | | ‘Stable’ = r6d2; |
| | | | | | ‘Worsening’ = r6d3; |
| | | | | | ‘N/A’ = r6d4 |
| Q7 | w7 | r7 | Q7a | w7a | ‘Yes’ = r7a1; ‘No’ = r7a2 |
| | | | Q7b | w7b | ‘Yes’ = r7b1; ‘No’ = r7b2; |
| | | | | | ‘N/A’ = r7b3 |
| | | | Q7c | w7c | ‘Intermittent’ = r7c1; |
| | | | | | ‘Persistent’ = r7c2 |
| | | | Q7d | w7d | ‘Improving’ = r7d1; |
| | | | | | ‘Stable’ = r7d2; |
| | | | | | ‘Worsening’ = r7d3; |
| | | | | | ‘N/A’ = r7d4 |
| Q8 | w8 | r8 | Q8a | w8a | ‘Yes’ = r8a1; ‘No’ = r8a2 |
| | | | Q8b | w8b | ‘Yes’ = r8b1; ‘No’ = r8b2; |
| | | | | | ‘N/A’ = r8b3 |
| | | | Q8c | w8c | ‘Intermittent’ = r8c1; |
| | | | | | ‘Persistent’ = r8c2 |
| | | | Q8d | w8d | ‘Improving’ = r8d1 ; |
| | | | | | ‘Stable’ = r8d2; |
| | | | | | ‘Worsening’ = r8d3; |
| | | | | | ‘N/A’ = r8d4 |
| Q9 | w9 | r9 | Q9a | w9a | ‘Yes’ = r9a1; ‘No’ = r9a2 |
| | | | Q9b | w9b | ‘Yes’ = r9b1; ‘No’ = r9b2; |
| | | | | | ‘N/A’ = r9b3 |
| | | | Q9c | w9c | ‘Intermittent’ = r9c1; |
| | | | | | ‘Persistent’ = r9c2 |
| | | | Q9d | w9d | ‘Improving’ = r9d1; |
| | | | | | ‘Stable’ = r9d2; |
| | | | | | ‘Worsening’ = r9d3; |
| | | | | | ‘N/A’ = r9d4 |
| Q10 | w10 | r10 | Q10a | w10a | ‘Yes’ = r10a1; ‘No’ = r10a2 |
| | | | Q10b | w10b | ‘Yes’ = r10b1; ‘No’ = r10b2; |
| | | | | | ‘N/A’ = r10b3 |
| | | | Q10c | w10c | ‘Intermittent’ = r10c1; |
| | | | | | ‘Persistent’ = r10c2 |
| | | | Q10d | w10d | ‘Improving’ = r10d1; |
| | | | | | ‘Stable’ = r10d2; |
| | | | | | ‘Worsening’ = r10d3; |
| | | | | | ‘N/A’ = r10d4 |
| Q11 | w11 | r11 | Q11a | w11a | ‘Yes’ = r11a1; ‘No’ = r11a2 |
| | | | Q11b | w11b | ‘Yes’ = r11b1; ‘No’ = r11b2; |
| | | | | | ‘N/A’ = r11b3 |
| | | | Q11c | wile | ‘Intermittent’ = r11c1; |
| | | | | | ‘Persistent’ = r11c2 |
| | | | Q11d | w11d | ‘Improving’ = r11d1; |
| | | | | | ‘Stable’ = r11d2; |
| | | | | | ‘Worsening’ = r11d3; |
| | | | | | ‘N/A’ = r11d4 |
| Q12 | w12 | r12 | Q12a | w12a | ‘Yes’ = r12a1; ‘No’ = r12a2 |
| | | | Q12b | w12b | ‘Yes’ = r12b1; ‘No’ = r12b2; |
| | | | | | ‘N/A’ = r12b3 |
| | | | Q12c | w12c | ‘Intermittent’ = r12c1; |
| | | | | | ‘Persistent’ = r12c2 |
| | | | Q12d | w12d | ‘Improving’ = r12d1; |
| | | | | | ‘Stable’ = r12d2; |
| | | | | | ‘Worsening’ = r12d3; |
| | | | | | ‘N/A’ = r12d4 |
| Q13 | w13 | r13 | Q13a | w13a | ‘Yes’ = r13a1; ‘No’ = r13a2 |
| | | | Q13b | w13b | ‘Yes’ = r13b1; ‘No’ = r13b2; |
| | | | | | ‘N/A’ = r13b3 |
| | | | Q13c | w13c | ‘Intermittent’ = r13c1; |
| | | | | | ‘Persistent’ = r13c2 |
| | | | Q13d | w13d | ‘Improving’ = r13d1; |
| | | | | | ‘Stable’ = r13d2; |
| | | | | | ‘Worsening’ = r13d3; |
| | | | | | ‘N/A’ = r13d4 |
| Q14 | w14 | r14 | Q14a | w14a | ‘Yes’ = r14a1; ‘No’ = r14a2 |
| | | | Q14b | w14b | ‘Yes’ = r14b1; ‘No’ = r14b2; |
| | | | | | ‘N/A’ = r14b3 |
| | | | Q14c | w14c | ‘Intermittent’ = r14c1; |
| | | | | | ‘Persistent’ = r14c2 |
| | | | Q14d | w14d | ‘Improving’ = r14d1; |
| | | | | | ‘Stable’ = r14d2; |
| | | | | | ‘Worsening’ = r14d3; |
| | | | | | ‘N/A’ = r14d4 |
| Q15 | w15 | r15 | Q15a | w15a | ‘Yes’ = r15a1; ‘No’ = r15a2 |
| | | | Q15b | w15b | ‘Yes’ = r15b1; ‘No’ = r15b2; |
| | | | | | ‘N/A’ = r15b3 |
| | | | Q15c | w15c | ‘Intermittent’ = r15c1; |
| | | | | | ‘Persistent’ = r15c2 |
| | | | Q15d | w15d | ‘Improving’ = r15d1; |
| | | | | | ‘Stable’ = r15d2; |
| | | | | | ‘Worsening’ = r15d3; |
| | | | | | ‘N/A’ = r15d4 |

The weights w6 to w15 of the items Q6 to Q15 may range from numerical values 1 to 4. In another embodiment of the present technique, the items Q6, Q9 to Q13 and Q15 are less important than the items Q7, Q8 and Q14, and therefore each of the items Q6, Q9 to Q13, and Q15 are assigned lower weights than each of the items Q7, Q8 and Q14. The scores, r6a1, r6a2, r6b1, r6b2, r6b3, . . . r15d1, r15d2, r15d3, and r15d4, as enlisted in Table 5 may range from numerical values 0 to 4.

The second group score may be calculated as:

$$Second Group score = {\sum }_{B = 6}^{15} wB * rB$$

wherein, B represents the item number between sixth and the fifteenth item from Table 2 or Table 5 i.e. Q6 and Q15, and wB represents the weight corresponding to that item, and rB represents the score of the response corresponding to that item.

When a given item between Q6 to Q15 includes a plurality of sub-items, for example as shown in Table 2 or Table 5, the item score for that item may be calculated based on sub-item weights and the scores of the corresponding predetermined responses to the sub-items. In one embodiment, when a given item between Q6 to Q15 includes a plurality of sub-items, for example as shown in Table 2 or Table 5, the item score for that item may be calculated by multiplying the sub-item weights and the scores of the corresponding selected responses to the sub-items and then averaging the products so obtained. For example, if the second group included item Q6 having four sub-items—Q6a, Q6b, Q6c and Q6d, as shown in Table 2 or Table 5, the item score for item Q6 may be calculated as follows:

Q6 Item score=¼*[w6a(r6a1 or r6a2)+w6b(r6b1 or r6b2 or r6b3)+w6c(r6c1 or r6c2)+w6d(r6d1 or r6d2 or r6d3 or r6d4)]

In the above equation, whether r6a1 or r6a2 is used depends on the response to the sub-item selected by the user, similarly, whether r6b1 or r6b2 or r6b3 is used and whether r6c1 or r6c2 is used and whether r6d1 or r6d2 or r6d3 or r6d4 is used depends on the response selected by the user for each of the sub-items Q6a, Q6b, Q6c and Q6d.

For each items Q7 to Q15, when included in the second group and having sub-items as shown in Tables 2 and 5, the item score for that item may be calculated as shown hereinabove for item Q6.

Thereafter, the second group score is determined based on the determined item scores of the items of the second group for example by adding the item scores of the items of the second group.

TABLE 6
Third Group—items, item weights, corresponding predetermined
responses and response scores

| | Item | Response | |
|---|---|---|---|
| | weight | Score | Corresponding predetermined |
| Items | (wC) | (rC) | responses and scores |
| Q16 | w16 | r16 | ‘No impact’ = r161; ‘Little |
| | | | impact’ = r162; ‘Moderate |
| | | | impact’ = r163; ‘Severe |
| | | | impact’ = r164; ‘Patient is |
| | | | immobile’ = r165 |
| Q17 | w17 | r17 | ‘No impact’ = r171; ‘Little |
| | | | impact’ = r172; ‘Moderate |
| | | | impact’ = r173; ‘Severe |
| | | | impact’ = r174; ‘Unable to |
| | | | self-care’ = r175 |
| Q18 | w18 | r18 | ‘No impact’ = r181; ‘Little |
| | | | impact’ = r182; ‘Moderate |
| | | | impact’ = r183; ‘Severe |
| | | | impact’ = r184; ‘Unable to |
| | | | complete daily activities’ = r185 |
| Q19 | w19 | r19 | ‘No impact’ = r191; ‘Little |
| | | | impact’ = r192; ‘Moderate |
| | | | impact’ = r193; ‘Severe |
| | | | impact’ = r194; ‘Unable to |
| | | | participate in hobbies and |
| | | | leisure activities’ = r195 |
| Q20 | w20 | r20 | ‘No impact’ = r201; ‘Little |
| | | | impact’ = r202; ‘Moderate |
| | | | impact’ = r203; ‘Severe |
| | | | impact’ = r204; ‘Unable to |
| | | | work’ = r205 |

The weights w16 to w20 of the items Q16 to Q20 may range from numerical values 1 to 4. In another embodiment of the present technique, the items Q16 and Q18 are more important than the items Q17, Q19 and Q20 and therefore each of the items Q16 and Q18 are assigned higher weights than each of the items Q17, Q19 and Q20. The scores, r161, r162, r163, r201, r202, r203, r204 and r205 as enlisted in Table 6 may range from numerical values 0 to 4.

The third group score may be calculated as:

$$Third Group score = {\sum }_{C = 16}^{20} wC * rC$$

wherein, C represents the item number between sixteenth and twentieth item from Table 3 or Table 6 i.e. Q16 and Q20, and wC represents the weight corresponding to that item, and rC represents the score of the response corresponding to that item.

In the above equation, rC is substituted with the score of the selected response for that item, for example when C=16, i.e. when the item is Q16, if the selected response is ‘No impact’ then rC which is r16 is substituted by r161; if the selected response is ‘Little impact’ then rC which is r16 is substituted by r162; if the selected response is ‘Moderate impact’ then rC which is r16 is substituted by r163; if the selected response is ‘Severe impact’ then rC which is r16 is substituted by r164; and if the selected response is ‘Patient is immobile’ then rC which is r16 is substituted by r165. Similarly, rC is also substituted with the score of the selected response for the items Q17 to Q20 when included in the third group, as explained hereinabove for rC of Q16.

The processor 30 also determines an EDSS group score based on an inputted EDSS score and a predetermined weight assigned for the EDSS query. The method of the present technique, after the step 150, includes a step 160 of determining, by the processor 30, the EDSS group score based on the inputted EDSS score and the predetermined weight assigned for the EDSS query.

The Expanded Disability Status Scale (EDSS) is a method of quantifying disability in multiple sclerosis. The EDSS is based on a neurological examination by a clinician, however a number of versions are presently available which enable patient self-administration, for example an online EDSS calculator that may be used by the patient or a physician to assess the EDSS score of the patient.

The EDSS quantifies disability in eight Functional Systems (FS) by assigning a Functional System Score (FSS) in each of these. The eight FS are: pyramidal (weakness or difficulty moving limbs), cerebellar (ataxia, loss of coordination or tremor), brainstem (problems with speech, swallowing and nystagmus), sensory (numbness or loss of sensations), bowel and bladder function, visual function, cerebral or mental functions and other.

TABLE 7
Expanded Disability Status Scale (EDSS)

| Score | Description |
|---|---|
| 1.0 | No disability, minimal signs in one FS |
| 1.5 | No disability, minimal signs in more than one FS |
| 2.0 | Minimal disability in one FS |
| 2.5 | Mild disability in one FS or minimal disability in two FS |
| 3.0 | Moderate disability in one FS, or mild disability |
| | in three or four FS. No impairment to walking |
| 3.5 | Moderate disability in one FS and more |
| | than minimal disability in several |
| | others. No impairment to walking |
| 4.0 | Significant disability but self-sufficient and |
| | up and about some 12 hours a day. |
| | Able to walk without aid or rest for 500 m |
| 4.5 | Significant disability but up and about much |
| | of the day, able to work a full day, |
| | may otherwise have some limitation |
| | of full activity or require minimal |
| | assistance. Able to walk without aid or rest for 300 m |
| 5.0 | Disability severe enough to impair full daily |
| | activities and ability to work a full |
| | day without special provisions. Able to |
| | walk without aid or rest for 200 m |
| 5.5 | Disability severe enough to preclude full |
| | daily activities. Able to walk without |
| | aid or rest for 100 m |
| 6.0 | Requires a walking aid—cane, crutch, etc.—to |
| | walk about 100 m with or without resting |
| 6.5 | Requires two walking aids—pair of canes, |
| | crutches, etc.—to walk about 20 m |
| | without resting |
| 7.0 | Unable to walk beyond approximately 5 m |
| | even with aid. Essentially restricted |
| | to wheelchair; though wheels self in |
| | standard wheelchair and transfers alone. |
| | Up and about in wheelchair some 12 hours a day |
| 7.5 | Unable to take more than a few steps. |
| | Restricted to wheelchair and may need |
| | aid in transferring. Can wheel self but |
| | cannot carry on in standard wheelchair |
| | for a full day and may require a motorized wheelchair |
| 8.0 | Essentially restricted to bed or chair |
| | or pushed in wheelchair. May be out of |
| | bed itself much of the day. Retains many |
| | self-care functions. Generally has |
| | effective use of arms |
| 8.5 | Essentially restricted to bed much of day. |
| | Has some effective use of arms |
| | retains some self-care functions |
| 9.0 | Confined to bed. Can still communicate and eat |
| 9 .5 | Confined to bed and totally dependent. |
| | Unable to communicate effectively or |
| | eat/swallow |
| 10.0 | Death due to MS |

In one embodiment of the system, the one or more processors is configured to determine the EDSS group score by: acquiring the EDSS score for the patient, generating a weighted EDSS score from the acquired EDSS score as a fraction of the maximum possible score of the Expanded Disability Status Scale i.e. the maximum score of 10 as shown in Table 7, and generating a reweighted EDSS score by multiplying the weighted EDSS score and the predetermined weight for the EDSS score.

The ‘weight’ assigned for the EDSS query is a numerical value that represent importance of the EDSS score vis-à-vis importance of the patient data query and age query in indicating SPMS state, for example a numerical value that reflects the influence of the EDSS score in deciding if the disease has progressed into SPMS from RRMS. Continuing one of the above examples wherein one of the numerals ‘1’, ‘2’ and ‘3’ may be assigned to different items, a total of five items (N1, N2, N3, N4, and N5—any 5 items selected from Q1 to Q20, e.g. one item—N1—from Q1 to Q5, two items—N2 and N3—from Q6 to Q15 and two items from Q16-Q20, as shown in Tables 1, 2, and 3)—each of the items N1 and N3 are assigned a weight of ‘1’, whereas item N2 is assigned a weight of ‘2’ and each of the items N4 and N5 are assigned a weight of ‘3’, the EDSS query may be assigned a weight of ‘2’. It may be noted however, that the weight of ‘2’ assigned to the EDSS query is for exemplary purposes only and other numerical values may be used as weight for the EDSS query in the present technique such as weight of ‘3’ or ‘4’. The weight assigned to the EDSS query in the present technique may be represented by an integer or by a fraction. In an embodiment, EDSS query may have variable assigned weights, for example, scores higher than one or more predetermined cut-offs may be assigned higher weights, such as EDSS scores≤4 may be assigned a weight of ‘1’, whereas a weight of ‘2’ may be assigned for EDSS scores ranging between 4.5-6.0 and a weight of ‘3’ may be assigned for EDSS scores>6.0—in short different cut-offs can be used to make different groupings within the EDSS scale and different weights can be assigned to these EDSS groupings, and the weight of the grouping into which the EDSS score of the patient falls is used as the weight assigned to the EDSS query.

In one embodiment, the weight assigned to the EDSS query may be between 1 and 3, for example w_{EDSS} and the reweighted EDSS score may be generated as follows:

$$RW \_ EDSS = \frac{{w}_{EDSS} * EDSS score}{10}$$

wherein, RW_EDSS is the reweighted EDSS score, w_{EDSS} is the weight assigned to the EDSS query, EDSS score is the value of the EDSS score determined for the patient, and denominator ‘10’ is used since the maximum score on the EDSS scale is 10.

In the present technique, the processor 30 allocates an inputted age of the patient to one of a plurality of predetermined age groups. Each of the predetermined age groups has an assigned predetermined score. The processor 30 determines an age score based on the score of the allocated age group and a predetermined weight assigned for the age query. In an embodiment of the system, the one or more processors is configured to determine the age score by multiplying the score of the allocated age group and the predetermined weight assigned for the age. The method of the present technique, after the step 170, includes a step 180 of determining, by the processor 30, the age score based on the inputted age and the predetermined weight assigned for the age query.

The ‘weight’ assigned for the age query is a numerical value that represent importance of the age score vis-à-vis importance of the patient data query and the EDSS query in indicating SPMS state, for example a numerical value that reflects the influence of the age score in deciding if the disease has progressed into SPMS from RRMS. Continuing one of the above examples wherein one of the numerals ‘1’, ‘2’ and ‘3’ may be assigned to different items, a total of five items (N1, N2, N3, N4, and N5—any 5 items selected from Q1 to Q20, e.g. one item—N1—from Q1 to Q5, two items—N2 and N3—from Q6 to Q15 and two items from Q16-Q20, as shown in Tables 1, 2, and 3)—each of the items N1 and N3 are assigned a weight of ‘1’, whereas item N2 is assigned a weight of ‘2’ and each of the items N4 and N5 are assigned a weight of ‘3’, the EDSS query is assigned a weight of ‘2’, the age query may be assigned a weight of ‘2’. It may be noted however, that the weight of ‘2’ assigned to the age query is for exemplary purposes only and other numerical values may be used as weight for the age query in the present technique such as weight of ‘3’ or ‘4’. The weight assigned to the age query in the present technique may be represented by an integer or by a fraction. Furthermore, different age groups may be assigned different scores.

The phrase ‘score’ as used in the present technique includes numerical value that represent importance of a given age group vis-à-vis importance of the other age groups for indicating the effect of age in progressing into SPMS state, for example a numerical value that reflects the influence of the age of the patient in further deciding if the disease has progressed into SPMS from RRMS. For example one of the numerals 0, 1, and 2 may be assigned to a different age groups, such as a first age group between 0-less than 10 years may be assigned a score of ‘0’, a second age group between 10-40 years may be assigned a score of ‘1’, and a third age group of greater than 40 years may be assigned a score of ‘2’ to reflect that the order of importance of the different age groups in indicating SPMS progression is—third age group (most important or most crucial) then second age group and finally the first age group, or in other words, if the age of the patient is such that it (the age of the patient) is allocated to the third age group then the likelihood of disease progression into SPMS is greater for that patient than if the age of the patient were such that it (the age of the patient) would be allocated to the first or the second age groups, and in another example if the age of the patient is such that it (the age of the patient) is allocated to the second age group then the likelihood of disease progression into SPMS is greater for that patient than if the age of the patient were such that it (the age of the patient) would be allocated to the first age group but the likelihood of disease progression into SPMS is lesser for that patient than if the age of the patient were such that it (the age of the patient) would be allocated to the third age group. It may be noted however, that the usage of scores for the age groups ranging from numerical values ‘0’, ‘1’, and ‘2’ is for exemplary purposes only and other numerical values may be used as scores for the different age groups in the present technique such as scores ranging from numerical values ‘0’, ‘1’, and ‘3’, scores ranging from numerical values ‘0’, ‘1’, ‘1.5″2’, ‘2.25’, ‘4’ and ‘5’, and so on and so forth. In other words, the different scores for the different age groups in the present technique may be represented by one or more integers, one or more fractions and a combination thereof.

In one embodiment of the present technique, age groups may be a first age group of less than 45 years of age having a score assigned as zero and a second age group may be greater than or equal to 45 years of age having a score assigned as two. In other embodiments other age groups may be used for example, a first age group of less than 40 years of age, a second age group of greater than or equal to 40 years and less than 60 years of age, and a third age group of greater than or equal to 60 years of age.

The processor 30 thereafter generates a total score based on the first group score, the second group score, the third group score, the EDSS group score and the age score. In another embodiment of the system, the first predetermined period, the second predetermined period and the third predetermined period are same. The method of the present technique, after the steps 140, 160 and 180, includes a step 190 of determining, by the processor 30, a total score based on the first group score, the second group score, the third group score as determined in the step 140, the EDSS group score as determined in the step 160 and the age score as determined in the step 180.

For example, the total score may be determined as:

Total score (TOTAL)=First group score+Second group score+Third group score+RW_EDSS+age score

Or, for example, the total score may be determined as:

Total score (TOTAL)=First group score+Second group score+Third group score+RW_T25FW+age score

Thereafter, the processor 30 determines the state of the MS or the status of progression of MS from RRMS to SPMS based on the total score. In one embodiment of the system, the one or more processors is configured to generate the total score by adding the first group score, the second group score, the third group score, the age score and optionally the EDSS group score (and/or the T25FW score). The method of the present technique, after the step 190, includes a step 200 of determining, by the processor 30, the state of MS i.e. either the first state for e.g. RRMS or the second state for e.g. SPMS, or the status of progression of MS from RRMS to SPMS, based on the total score as determined in the step 190.

The determination that the MS is still in RRMS state is done by the one or more processors by comparing the total score to a first threshold score for example if the total score is below the first threshold score. The determination that the MS has progressed from RRMS state to SPMS state is done by the one or more processors by comparing the total score to a second threshold score for example if the total score is equal to or above the second threshold score. The threshold scores, i.e. the first and the second threshold scores, are predetermined numerical values.

The first threshold and the second threshold scores may be the same and thus present a single threshold score which when reached by the total score or surpassed by the total score is indicative of SPMS state. Alternatively, when the total score does not reach or surpass the single threshold score indicates that MS has not progressed from RRMS to SPMS and the patient remains in RRMS state.

For example, when each item of the plurality of items, the EDSS query and the age query is assigned a weight between 1 and 3, and each corresponding predetermined response a score between 0 and 4, the maximum possible total score is 176 (Q1 to Q20, EDSS query and age query have different weights but assuming, for exemplary purpose, an average weight to be ‘2’ and assuming each item has response with score ‘4’) and the single threshold score is 90, and then if the total score is below 90 the processor is configured to determine the status of progression as—the MS is still in RRMS state and has not progressed to SPMS state, however if the total score is greater than or equal to 90 the processor is configured to determine the status of progression as—the MS has progressed from RRMS state to SPMS state.

The first threshold and the second threshold scores may be different from each other. In such a case, the processor determines a status of transitioning MS in progression from RRMS to SPMS based on comparing the total score with the first and/or the second threshold scores and determining whether the total score is equal to, greater than or less than the first and/or the second threshold scores. In one embodiment, the first and the second threshold scores are such that: (a) if the total score is equal to or less than the first threshold score—the total score is indicative of the patient still remaining in RRMS state i.e. the MS of the patient has not progressed from RRMS to SPMS, (b) if the total score is equal to or greater than the second threshold score—the total score is indicative that the patient has progressed from RRMS to SPMS i.e. the patient is in SPMS state, and (c) if the total score is between the first and the second threshold scores—the total score is indicative that there is a calculated or specific likelihood that the patient is in transition i.e. progressing from RRMS to SPMS i.e. the patient is in ‘in-transition from RRMS to SPMS’ state, on the date on which the determination using the method of the present technique is performed.

For example, when each item of the plurality of items, the EDSS query and the age query is assigned a weight between 1 and 3, and each corresponding predetermined response a score between 0 and 4, the maximum possible total score is 176 (Q1 to Q20, EDSS query and age query have different weights but assuming, for exemplary purpose, an average weight to be ‘2’ and assuming each item has response with score ‘4’), the first threshold score is 90 and the second threshold score is 110, then if the total score is equal to or below 90 the processor is configured to determine the state of MS or the status of progression as—the MS is still in RRMS state and has not progressed to SPMS state, if the total score is equal to or greater than 110 the processor is configured to determine the state of MS or the status of progression as—the MS has progressed from RRMS state to SPMS state i.e. the state of MS is SPMS state, whereas if the total score is greater than 90 but below 110 the processor is configured to determine the state of MS or the status of progression as—‘in-transition from RRMS to SPMS’—a progression from RRMS to SPMS is underway. The ‘in-transition from RRMS to SPMS state’ may also indicate that there is 80 percent likelihood that MS will completely transition from RRMS state to SPMS state in a predetermined period, for example in 6 months from the date when the technique is being performed.

A plurality of different threshold scores may be used to indicate different likelihoods of progression into SPMS, for example three threshold scores—T1, T2 and T3 may be used. The plurality of different threshold scores, i.e. for example the threshold scores T1, T2 and T3, are different from each other. If the total score is less than T1 it is determined that the status of progression of MS from RRMS to SPMS is that the MS still remains in RRMS and has not progressed to SPMS i.e. the patient is in ‘RRMS’ state, whereas if the if the total score is between T1 and T2 it is determined that the status of progression of MS from RRMS to SPMS is—‘in-transition from RRMS to SPMS’ state—that it is likely that the MS is in progression from RRMS to SPMS for example there is a 50 percent chance that the MS is in progression from RRMS to SPMS, whereas if the total score is between T2 and T3, it is determined that the status of progression of MS from RRMS to SPMS is—‘in-transition from RRMS to SPMS’ state—that it is highly likely that the MS is in progression from RRMS to SPMS for example there is a 80 percent chance that the MS is in progression from RRMS to SPMS. If the total score greater than T3 it is determined that the status of progression of MS from RRMS to SPMS is that the MS is in SPMS state. It may be appreciated by one skilled in the art that the number of threshold scores and the different likelihoods associated with each of the threshold scores for example the different percentages (i.e. 50 percent and 80 percent) used in the hereinabove are for exemplary purpose only, and that the number of threshold scores and the different likelihoods associated with each of the threshold scores for example the different percentages may be different from the used example values.

The single threshold value, or the first and the second threshold value, or the different thresholds are set in advance and may be predetermined empirically. The threshold value or values are determined as a result of patient group studies and/or physician group studies and may include clinical studies. Thresholds/cut-off are defined by statistical analyses, such as Receiver Operating Characteristic (ROC) curve analyses to determine the best possible sensitivity (true positive) and specificity (true negative) i.e. to determine optimal sensitivity and specificity.

Optionally, the total score so determined or generated by the processor 30 may be standardized, for example by using the following equation:

Standardized total score=(total score/maximum possible total score)*100

When the present technique uses standardized total score, the threshold value or values may be expressed as percentages.

Finally, in the present technique, the state of MS or the status of progression of MS from RRMS to SPMS so determined by the processor is outputted by the output means 40. The method of the present technique, after the step 200, includes a step 210 of outputting, by the output means, the state of MS or the status of progression of MS from RRMS to SPMS, so determined in the step 200.

The status is outputted for example as shown in the example of FIG. 7 via visual cues, for example by displaying a percentage and/or a text message 99 on the display, and/or by using symbol based system such as by different colored spots 90a, 90b, 90c e.g. a green spot or light 90a, a yellow spot or light 90b, and a red spot/or light 90c that indicate different stages of the progression for example the spot 90a may be indicative of no progression determined or least likelihood of progression from RRMS to SPMS i.e. the state of MS is ‘RRMS’ state, the spot 90b may be indicative of a higher likelihood of progression from RRMS to SPMS or that the state of MS is ‘in-transition from RRMS to SPMS’ state, whereas the spot 90c may be indicative of highest likelihood of progression from RRMS to SPMS i.e. the state of MS is in SPMS' state. As depicted in FIG. 7 a highlighting effect 91 may be used to indicate the status of progression by highlighting one of the spots 90a, 90b, 90c, whichever applicable. In one embodiment, different colored spots 90a, 90b, 90c may indicate different states of the MS for example the spot 90a (e.g. a green spot) may be indicative of RRMS state, the spot 90b (e.g. a yellow spot) may be indicative of ongoing transition from RRMS to SPMS i.e. ‘in-transition from RRMS to SPMS’ state, whereas the spot 90c (e.g. red spot) may be indicative of SPMS state. As depicted in FIG. 7 a highlighting effect 91 may be used to indicate the state of MS or the status of progression by highlighting one of the spots 90a, 90b, 90c, whichever applicable.

FIGS. 8A-8E depict screenshots of another embodiment of the GUI 12 in accordance with aspects of the present technique. As depicted in FIG. 8A the GUI 12 has the field for providing an age of the patient, in which the age of the patient has been provided as ‘45’ in the example of FIG. 8A and the field for providing an EDSS score of the patient, in which an existing/recently acquired EDSS score has been provided as ‘7.5’ in the example of FIG. 8A. In the GUI depicted in FIG. 8A the technique is presented as an application or web-based tool, and has been referred to as ‘ProDec: MS Progression Detection Tool’. Responses to the different items, age query, EDSS query, etc. may be provided by entering numbers and digits using a physical or a virtual keyboard, or by selecting from drop-down menu, or by onscreen slider function as shown for the EDSS score inputting, or by other types tactile inputs or inputs such as voice command. As depicted in FIG. 8B, all the items of the first group, along with the corresponding responses are displayed at the same time in a single page, whereas as depicted in FIG. 8C, all the items of the second group, along with their corresponding responses are displayed at the same time in a single page, and as depicted in FIG. 8D, all the items of the third group, along with their corresponding responses are displayed at the same time in a single page. The user can, thus, input responses to all the items of the first group by selecting from the responses for each item of the first group displayed simultaneously. Similarly, the user can input responses to all the items of the second group by selecting from the responses for each item of the second group displayed simultaneously. Similarly, the user can input responses to all the items of the third group by selecting from the responses for each item of the third group displayed simultaneously. FIG. 8E provides the ‘result’ i.e. the state of MS.

FIGS. 9A-9K depict screenshots of another embodiment of the GUI 12 in accordance with aspects of the present technique. In the GUI 12 depicted in FIGS. 9A-9K the technique is presented as an application, e.g. mobile application or computer application, or web-based tool, and has been referred to as ‘MSProDetect: MS Progression Detection Tool’, hereinafter simply referred to as the tool. In the example of FIGS. 9A-9K, references to items or sub-items, i.e. item or sub-items numbers and item or sub-item details, have been made in accordance with Tables 1, 2 and 3, however it may be noted, that item number i.e. Q1, Q2, etc. and item details may be different than as shown in Tables 1-3, i.e. for example although Table 2 shows item number Q6 corresponds to ‘Visual symptoms experienced in the past six months’ and item number Q10 corresponds to ‘pain experienced in the past six months’, and the same order or mapping of the items have been used in the embodiment of FIGS. 9A-9K, in another embodiment the order or mapping of the items may be different—for example in another embodiment item number Q6 may correspond to ‘pain experienced in the past six months’ and item number Q10 may correspond to ‘Visual symptoms experienced in the past six months’.

FIG. 9A depicts a screenshot of the tool at the start of the tool or at a point when the method of the present technique is initiated, i.e. no responses or inputs have been provided at this stage. As depicted in FIG. 9A the GUI 12 has the field 501 for providing an age of the patient i.e. “Patient age” in FIG. 9A, to which the age of the patient has to be provided for example by entering through a physical keyboard or a virtual keyboard. The GUI 12 also has the field 502 for providing an EDSS score of the patient, in which an existing/recently acquired EDSS score has to be provided, for example by moving the slider 502s (positioned at “0” in FIG. 9A) to a position reflecting the EDSS score of the patient, and thus the present technique is continued with using the EDSS score e.g. as discussed in Example 2A hereinbelow. If the EDSS score of the patient is not available, or is desired by the user (such as physician) of the present technique to be not used for some reason for example if the EDSS score unacceptable or is older than a predetermined period, then the user may select “NA” (i.e. not applicable) option, and thereafter the present technique is continued without using the EDSS score, e.g. as discussed in Example 2B hereinbelow. Furthermore, as shown in FIG. 9A, the GUI 12 displays the items Q1, Q4 or queries Q1, Q4 of the first group and the corresponding predetermined responses to select from. The user can select the applicable response for the item Q1 i.e. ‘Yes’ or ‘No’ whichever is applicable and similarly can select the applicable response for the item Q4 i.e. ‘Yes’ or ‘No’ whichever is applicable.

FIG. 9B shows a screenshot according to the present technique, subsequent to the screenshot of FIG. 9A. As shown in FIG. 9B, the age of the patient has been inputted in the field 501 as ‘54’ i.e. fifty-four, and the EDSS score has been entered or inputted as ‘4’ by sliding, on the display, the slider 502s to a position corresponding to EDSS score of ‘4’. As shown in FIG. 9B, when the response selected by the user from the corresponding predetermined responses of the item Q1 is ‘Yes’, further items or queries, that may be related to the item Q1, of the first group appear or get displayed, for example, as shown in FIG. 9B the items Q2 and Q3 which are related to item Q1 get displayed when the response to the item Q1 is selected as ‘Yes’. Along with the items Q2 and Q3, the corresponding predetermined responses for each of the items Q2 and Q3 are also displayed. The user can then choose or select the applicable responses for the items Q2 and Q3, for example by selecting the on-screen icons pertaining to the applicable responses for the items.

FIG. 9C shows a screenshot according to the present technique, subsequent to the screenshot of FIG. 9B. As shown in FIG. 9C, the user has selected applicable responses for the items Q2 and Q3 by selecting, for example by touching or selecting on-screen icons of the items, the applicable response from the corresponding predetermined responses to the items Q2 and Q3. FIG. 9C depicts the response—“2” selected for the item Q2 and the response—“Nearly full recovery (75%)” selected for the item Q3. FIG. 9C also depicts that the response selected by the user from the corresponding predetermined responses of the item Q4 is ‘Yes’, and that further item(s), that may be related to the query or the item Q4, of the first group appear or get displayed, for example, as shown in FIG. 9C the item Q5 and the corresponding predetermined responses for the item Q5 are displayed. The user can then choose or select the applicable responses for the item Q5.

FIG. 9D shows a screenshot according to the present technique, subsequent to the screenshot of FIG. 9A, depicting a progression of the present technique different from the progression depicted through the FIGS. 9B and 9C. As shown in FIG. 9D, the age of the patient has been inputted in the field 501 as ‘54’ and the EDSS score has been entered or inputted as ‘4’ by sliding the slider 502s to a position corresponding to EDSS score of ‘4’. However, in contrast to as shown in FIG. 9B, the response selected by the user from the corresponding predetermined responses to the item Q1 is ‘No’, and thus no further items or queries related to the item Q1 of the first group appear or get displayed, as shown in FIG. 9D, the items Q2 and Q3 which are related to item Q1 do not get displayed when the response to the item Q1 is selected as ‘No’. Consequently, the corresponding predetermined responses of the items Q2 and Q3 are also not displayed. The user can therefore not choose or select the applicable responses for the items Q2 and Q3. As depicted in FIG. 9D, however, the response selected by the user from the corresponding predetermined responses of the item Q4 is ‘Yes’, same as FIG. 9C, and thus further item(s) or queries, that may be related to the query or item Q4, of the first group appear or get displayed, for example, as shown in FIG. 9D the item Q5 and the corresponding predetermined responses for the item Q5 are displayed, even though the items Q2 and Q3 are not displayed. The user can then choose or select the applicable responses for the item Q5, and as shown in FIG. 9D the user has selected the response ‘Signs of new activity’ out of the corresponding responses of—‘No signs of new activity’ (same as response ‘No’ shown in Table 1) and ‘Signs of new activity’ (same as response ‘Yes’ shown in Table 1).

FIG. 9E shows a screenshot according to the present technique, subsequent to the progression of the method of the present technique depicted by the screenshots of FIG. 9A then 9B and then 9C or subsequent to the progression of the method of the present technique depicted by the screenshots of FIG. 9A then 9D, i.e. depicting further progression of the present technique either after the stage of FIG. 9C or FIG. 9D. It may be noted that not all the screenshots depicting continuous method or tool of the present technique are depicted, for example in the method of the present technique at the stage or step depicted by the screenshot of FIG. 9C, the user still needs to select an applicable response for the item Q5 since none of the corresponding responses for the item Q5 are yet selected in example screenshot of FIG. 9C. Before proceeding to the stage of the method depicted by FIG. 9E, the user may submit or input the responses i.e. the provide the selection of the responses to the one or more processors by selecting an on-screen tab 503, as shown in FIGS. 9A-9D.

FIG. 9E depicts a screenshot showing the items of the second group. As shown in FIG. 9E all the items Q6-Q15 are displayed, however initially (compared to FIGS. 9F-H) only sub-items 6a, 7a, 8a, 9a, 10a, 11a, 12a, 13a, 14a, 15a are displayed, for example in form of on-screen tabs, for the items Q6-Q15. The dotted area ‘a’ marked in FIG. 9E represents sub-item details applicable for each of the items Q6-Q15, as shown in Table 2. Similarly, the dotted areas ‘c’ and ‘d’ marked in FIG. 9E represent sub-item details applicable for each of the items Q6-Q15, as shown in Table 2. The user can select which items of the second group are to be included in the calculation of the second group score by selecting, for example by touching or selecting the on-screen displayed icons of the items to be selected i.e. for example by selecting or touching the on-screen tabs Q6a to Q15a.

FIG. 9F shows a screenshot according to the present technique, subsequent to the screenshot of FIG. 9E. The user, as an example, has selected the item Q6a (i.e. the icon for the item Q6 under the dotted area a), and thereafter the corresponding responses for the remaining sub-items Q6b, Q6c and Q6d are displayed for the user to select from. Similarly, the user has selected the sub-items Q9a (i.e. the icon for the item Q9 under the dotted area a), Q13a (i.e. the icon for the item Q13 under the dotted area a), and Q15a (i.e. the icon for the item Q15 under the dotted area a) and thereafter the corresponding responses for the remaining sub-items Q9b-Q9d, Q13b-Q13d and Q15b-Q15d are displayed for the user to select from, for example by selecting the icons corresponding to the applicable responses.

FIG. 9G shows a screenshot according to the present technique, subsequent to the screenshot of FIG. 9F. The user, as an example, has selected the applicable responses for the sub-items Q6b, Q6c, and Q6d—as ‘Yes’, ‘Persistent’ and ‘Improving’, respectively, (selection of ‘Persistent’ is implicit since the user selected ‘Improving’ under ‘Persistent’) and for the sub-item Q9b as ‘No’. Since the response to sub-item Q9b is ‘No’—the need or requirement of selecting or inputting responses for the sub-items Q9c and Q9d becomes moot so no selection of applicable response for the sub-items Q9c and Q9d is necessary, or in other words the sub-items Q9c and Q9d are rendered moot. As shown in FIG. 9G the user is still required to provide responses, i.e. select applicable responses, for the sub-items Q13b-Q13d and for the sub-items Q15b-Q15d.

FIG. 9H shows a screenshot according to the present technique, wherein the user, as an example, has selected the applicable responses for each of the sub-items of each of the items Q6 to Q15.

FIG. 9I depicts a screenshot showing the items of the third group and may be subsequently displayed after the screenshot of FIG. 9H. Before proceeding to the stage of the method depicted by FIG. 9I, the user may submit or input the responses i.e. the provide the selection of the responses to the one or more processors by selecting an on-screen tab 504, as shown in FIGS. 9E-9H. As shown in FIG. 9I along with the items Q16 to Q20, the corresponding responses for each of the items Q16 to Q20 are displayed for the user to select from i.e. are displayed for the user so that the user can select applicable responses to each of the items Q16-Q20.

FIG. 9J shows a screenshot according to the present technique, wherein the user, as an example, has selected the applicable responses for each of the items Q16 to Q20. Before proceeding to further in the method of the present technique, the user may submit or input the responses i.e. the provide the selection of the responses to the one or more processors by selecting an on-screen tab 505, as shown in FIGS. 9I and 9J.

FIG. 9K provides the ‘result’ i.e. the state of MS. As shown in FIG. 9K the state of MS has not progressed to SPMS—shown in the FIG. 9K as ‘Unlikely that the patient has progressed to SPMS’. Thus, the state of MS may be in the RRMS state, or alternatively, the state of MS may be in in-transition from RRMS to SPMS state. The reference signs 90a, 90b, 90c and 91 used in FIG. 9K may be understood similarly to as used in FIG. 7. FIG. 9K therefore shows that the state of MS is RRMS state.

It may be noted that, although, the tool of the embodiment of FIGS. 9A-9K has been presented as progressing from FIGS. 9A-9D, then FIGS. 9E-9H, and then FIGS. 9I-9J to provide the result in FIG. 9K, it may be appreciated by one skilled in the art that the tool may be structured such that the order of progression is different than FIGS. 9A-9D to FIGS. 9E-9H to FIGS. 9I-9J, for example the order of progression may be FIGS. 9E-9H, then FIGS. 9A-9D, and then FIGS. 9I-9J to provide the result in FIG. 9K. Alternatively, the order of progression may be FIGS. 9E-9H, then FIGS. 9I-9J, and then FIGS. 9A-9D to provide the result in FIG. 9K In short, the steps depicted via the FIGS. 9A-9D, the steps depicted via the FIGS. 9E-9H, and the steps depicted via the FIGS. 9I-9J may be arranged in any order to arrive at the result of the present technique as depicted by FIG. 9K.

The invention further concerns a method for treating multiple sclerosis (MS) in a patient in need thereof, wherein in the second step an MS therapeutic is administered to the patient based on the state so determined. In an embodiment, the first state is RRMS and/or the second state is SPMS.

Generally, the invention is not limited to a specific MS therapeutic. Examples of suitable MS therapeutic are injectable therapeutics like interferon beta-1a, interferon beta-1b, which can optionally be pegylated, glatiramer acetate; infused therapeutics like alemtuzumab, mitoxantrone, ocrelizumab, natalizumab; oral therapeutics like teriflunomide, dimethyl fumarate, fingolimod and/or siponimod.

In an embodiment of the method for treating MS, the MS therapeutic is an sphingosine-1-phosphate (S1P) receptor modulator. In an embodiment, the sphingosine-1-phosphate (S1P) receptor modulator is administered orally.

In another embodiment of the method for treating MS, the S1P receptor modulator is fingolimod or a pharmaceutically acceptable salt thereof. In an embodiment of the method, the patient is administered fingolimod if the state of MS in the patient is the first state, for example the first state is RRMS.

“Fingolimod” as used herein is understood to comprise the compound of formula (I)

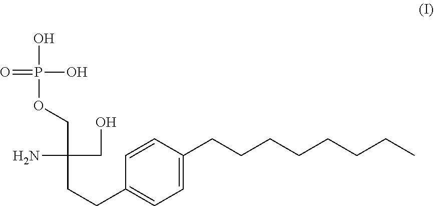 [SMILES: CCCCCCCCc1ccc(CCC(N)(CO)COP(=O)(O)O)cc1]

as well as the pharmaceutically acceptable salts, co-crystals, polymorphs, solvates and/or hydrates thereof. Fingolimod as used herein has the IUPAC-name 2-amino-2-[2-(4-octylphenyl)ethyl]propane-1,3-diol (also being abbreviated as FTY720).

Examples of pharmaceutically acceptable salts or co-crystals of fingolimod include salts or co-crystals with acids, such as hydrochloric acid, acetic acid, fumaric acid, malic acid or the like. In an embodiment, fingolimod is used in the hydrochloride salt form.

In an embodiment, fingolimod is administered in a daily dosage of about 0.1 to about 1.5 mg, for example, in a daily dosage of about 0.25 or about 0.5 mg, for example, about 0.5 mg, based on fingolimod free base. Said daily dose can be referred to as “maintenance dose”.

In yet another embodiment of the method for treating MS, the S1P receptor modulator is siponimod or a pharmaceutically acceptable salt thereof. In a further embodiment of the method, the patient is administered siponimod if the state of MS in the patient is the second state, for example the second state is SPMS.

In another embodiment of the method, when the state of MS includes a third state wherein the third state is ‘in-transition from RRMS to SPMS’ state, the patient is administered siponimod if the state of MS in the patient is determined to be the third state.

“Siponimod” as used herein is understood to comprise the compound of formula (I)

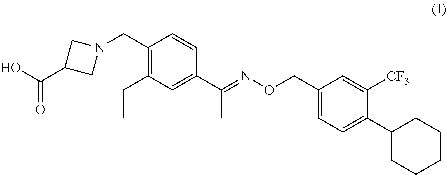 [SMILES: CCc1cc(/C(C)=N/OCc2ccc(C3CCCCC3)c(C(F)(F)F)c2)ccc1CN1CC(C(=O)O)C1]

as well as the pharmaceutically acceptable salts, co-crystals, polymorphs, solvates and/or hydrates thereof. Siponimod, as used herein, has the IUPAC-name 1-{4-[1-((E)-4-cyclohexyl-3-trifluoromethyl-benzyloxyimino)-ethyl]-2-ethyl-benzyl}-azetidine-3-carboxylic acid (lab-code BAF312).

Examples of pharmaceutically acceptable salts or co-crystals of siponimod include salts or co-crystals with acids, such as hydrochloric acid, acetic acid, fumaric acid, malic acid or the like. In an embodiment, siponimod is used in the hemifumarate salt form. In another embodiment, siponimod is used in the form of a co-crystal of siponimod free base and fumaric acid, wherein, for example, the molar ratio of fumaric acid:siponimod free base is about 0.5.

In an embodiment, siponimod is administered in a daily dosage of about 0.1 to about 5.0 mg, for example, in a daily dosage of about 2.0 mg, based on siponimod free base. Said daily dose can be referred to as “maintenance dose”.

In a further embodiment of the present invention, the patient has experienced a titration regimen before administering the maintenance dose. In an embodiment, the titration regimen includes administering of 0.25 mg of siponimod at day 1, 0.25 mg at day 2, 0.5 mg at day 3, 0.75 mg at day 4 and 1.25 mg at day 5.

### EXAMPLES

Hereinafter examples of the present technique have been described—namely, Example 1, Example 2 and Example 3. The features and explanations and embodiments described hereinabove may apply to the examples, and the examples can be accordingly modified.

In each of the examples—Example 1 and Example 2, referring to Table 1, Table 2 and Table 3, the first group may include one or more or all of the items Q1 to Q5 from the items Q1 to Q5 of Table 1, the second group may include one or more or all of the items Q6 to Q15 from the items Q6 to Q15 of Table 2, and the third group may include one or more or all of the items Q16 to Q20 from the items Q16 to Q20 of Table 3, respectively. Furthermore, the second group includes all the sub-items, shown in Table 2, corresponding to the one or more of the items Q6 to Q15 included in the second group.

For example, in each of the examples—Example 1 and Example 2 and Example 3, the first group may include all the items Q1 to Q5 of Table 1, the second group may include all the items Q6 to Q15 and all the sub-items for each of the items Q6 to Q15 of Table 2, and the third group may include all the items Q16 to Q20 of Table 3.

### Example 1

Table 4A, Table 5A and Table 6A provide the weights of the items Q1-Q20 and the scores of the corresponding responses to each of the items Q1-Q20. Table 5A further includes the weights of the sub-items for each of the items Q6-Q15 and the scores of the corresponding responses to each of the sub-items. Table 4A, Table 5A and Table 6A correspond to Table 4, Table 5 and Table 6, respectively. Optionally, Table 4A, Table 5A and Table 6A show maximum possible scores for each of the items, and the sub-items where applicable. For items having sub-items—the maximum possible scores for the item is total of the maximum possible score of the sub-items in that item. The maximum possible group scores have also been indicated, assuming that the first group includes all of Q1 to Q5, the second group includes all of Q6 to Q15 and the third group includes all of Q16 to Q20.

TABLE 4A
First Group—items, item weights, corresponding predetermined responses
and response scores

| | | | Corresponding | Maximum |
|---|---|---|---|---|
| | | | predetermined responses | possible |
| | Item | Response | and scores (i.e. r11, | score |
| | weight | Score | r12, r21, .... r51, r52 as enlisted | (for first |
| Items | (wA) | (rA) | in Table 4 above) | Group = 11) |
| Q1 | w1 = 3 | r1 | ‘Yes’ = 0; ‘No’ = 1 | 3 |
| Q2 | w2 = 2 | r2 | ‘1’ = 0; ‘2’ = 1; ‘3 or more’ = 2 | 4 |
| Q3 | w3 = 2 | r3 | ‘Full recovery (100%)’ = 0; | 4 |
| | | | ‘Nearly full recovery | |
| | | | (75%)’ = 0; ‘Partial recovery | |
| | | | (50%)’ = 1; ‘Little | |
| | | | recovery (25%)’ = 1; | |
| | | | ‘No recovery (0%)’ = 2 | |
| Q4 | w4 = 1 | r4 | ‘Yes’ = 0; ‘No’ = 0 | 0 |
| Q5 | w5 = 3 | r5 | ‘Yes’ = 1; ‘No’ = 0 | 3 |

It may be noted, for this example or for Example 2 or generally for the description, that for any of the groups the maximum possible group score is not necessarily a total of the maximum possible scores of the items contained in the group, and rather dependent on the nature of items of the group, for example in Table 4A—if response to Q1 is ‘No’ then response to Q2 and Q3 cannot reflect that the patient had ‘2’ or ‘3 or 4’ relapses or that the patient ‘Fully recovered’—since Q1 response indicated that there were no relapses to begin with.

TABLE 5A
Second Group—items, item weights, corresponding predetermined
responses and response scores.

| | | | | | Maximum |
|---|---|---|---|---|---|
| | | | | | possible score |
| | | | | Score of the | (for second |
| | | | | sub-items | group = 14, |
| | | | | (i.e. r6a1, r6a2, | based on the |
| | | | | r6b1, r6b2, | equation Ex1 |
| | | | | r6b3, r6c1 . . . | for |
| | Item | | | r15d3, r15d4 | calculating |
| | weight | | Weights | as enlisted | second group |
| | (for each | | of sub- | in Table 5 | score for this |
| Items | item) | Sub-item | item | above) | example) |
| Q6, Q9, | 1 | a | — | Yes = 1, No = 0 | |
| Q10, | (i.e. each | b if ‘Yes’ | 1 | Yes = 0, No = 1, | 1 |
| Q11, | of w6, | for a | | N/A = 0 | |
| Q12, | w9 to | c if ‘Yes’ | 3 | Intermittent = 0, | 3 |
| Q13, | w13, | for a | | Persistent = 1 | |
| Q15 | w15 = | d if ‘Yes’ | 3 | Improving = 0, | 3 |
| | 1) | for a | | Stable = 0, | |
| | | | | Worsening = 1, | |
| | | | | N/A = 0 | |
| Q7, Q8, | 2 | a | — | Yes = 1, No = 0 | |
| Q14 | (i.e. each | b if ‘Yes’ | 1 | Yes = 0, No = 1, | 2 |
| | of w7, | for a | | N/A = 0 | |
| | w8 and | c if ‘Yes’ | 3 | Intermittent = 0, | 6 |
| | w14 = 2) | for a | | Persistent = 1 | |
| | | d if ‘Yes’ | 3 | Improving = 0, | 6 |
| | | for a | | Stable = 0, | |
| | | | | Worsening = 1, | |
| | | | | N/A = 0 | |

Note:
Sub-item ‘a’ (i.e. Q6a, Q7a, Q8a, Q9a, Q10a, Q11a, Q12a, Q13a, Q14a, Q15a) in items Q6 to Q15 is used only as an indication of how many symptoms are present and therefore is not weighted.

TABLE 6A
Third Group—items, item weights, corresponding predetermined
responses and response scores

| | | | | Maxi- |
|---|---|---|---|---|
| | | | Corresponding predetermined | mum |
| | | | responses and | possible |
| | | Res- | scores | score |
| | Item | ponse | (i.e. r161, r162, | (for third |
| | weight | Score | r163, . . . r204, r205, r206 | Group = |
| Items | (wC) | (rC) | as enlisted in Table 6 above) | 14) |
| Q16 | 2 | r16 | ‘No impact’ = 0; ‘Little impact’ = 1; | 4 |
| | | | ‘Moderate impact’ = 1; | |
| | | | ‘Severe impact’ = 2; | |
| | | | ‘Patient is immobile’ = 2 | |
| Q17 | 1 | r17 | ‘No impact’ = 0; ‘Little impact’ = 1; | 2 |
| | | | ‘Moderate impact’ = 1; | |
| | | | ‘Severe impact’ = 2; | |
| | | | ‘Unable to self-care’ = 2 | |
| Q18 | 2 | r18 | ‘No impact’ = 0; ‘Little impact’ = 1; | 4 |
| | | | ‘Moderate impact’ = 1; | |
| | | | ‘Severe impact’ = 2; | |
| | | | ‘Unable to complete | |
| | | | daily activities’ = 2 | |
| Q19 | 1 | r19 | ‘No impact’ = 0; ‘Little impact’ = 1; | 2 |
| | | | ‘Moderate impact’ = 1; | |
| | | | ‘Severe impact’ = 2; | |
| | | | ‘Unable to participate in | |
| | | | hobbies and leisure | |
| | | | activities’ = 2 | |
| Q20 | 1 | r20 | ‘No impact’ = 0; ‘Little impact’ = 1; | 2 |
| | | | ‘Moderate impact’ = 1; | |
| | | | ‘Severe impact’ = 2; | |
| | | | ‘Unable to work’ = 2 | |

Table 7A represents the weights and scores for the EDSS and age.

TABLE 7A
EDSS and age

| | |
|---|---|
| Reweighted | Reweighted to be out of a total of 3, i.e. EDSS query |
| EDSS | given a question weight of 3, |
| score | |
| RW_EDSS | $\frac{3 * EDSS score}{10}$ |
| Age score | Score either 0 or 2, i.e. given a question weight of 2 |
| | $\{ \begin{matrix}age < 45 , & 0 \\ age \geq 45 , & 2\end{matrix}$ |

### Calculation of Total Score (TOTAL):

The item score for each item Q1-Q20, when all the items Q1 to Q20 are included in the first, the second and the third groups, is determined or calculated by multiplying the item weight with the corresponding response selected for that item. In case of items of the second group, i.e. items Q6 to Q15, the sub-item scores are calculated to indicate the item score.

First Group score=(w1*r1)+(w2*r2)+(w3*r3)+(w4*r4)+(w5*r5)

When w1-w5 are substituted with their weights obtained from Table 4A,

First Group score=(3*r1)+(2*r2)+(2*r3)+(1*r4)+(3*r5)

wherein r1-r5 are the responses, which are then substituted by scores r11, r12, etc. as selected and then values of r11, r12, etc. are substituted from Table 4A to obtain or determine the first group score. For example if the response for the item Q1 is chosen or selected as ‘Yes’, the score r1 is substituted by r11 (as shown in Table 4) and thereafter the score r11 is substituted by value or score zero i.e. ‘0’ (as shown in Table 4A), similarly for example if the response for the item Q3 is chosen or selected as ‘No recovery’, the score r3 is substituted by r35 (as shown in Table 4) and thereafter the score r35 is substituted by value or score of two i.e. ‘2’ (as shown in Table 4A).

Second group score, i.e. equation Ex1 as referred to in Table 5A

$$= \frac{\begin{matrix}[ w 6 ( w 6 b * score\_selected response for Q 6 b + \\ w 6 c * score\_selected response for Q 6 c + \\ w 6 d * score\_selected response for Q 6 d ) + \\ w 7 ( w 7 b * score\_selected response for Q 7 b + \\ w 7 c * score\_selected response for Q 7 c + \\ w 7 d * score\_selected response for Q 7 d ) + … \\ … + w 15 ( w 15 b * score\_selected response for Q 15 b + \\ w 15 c * score\_selected response for Q 15 c + \\ w 15 d * score\_selected response for Q 15 d ) ]\end{matrix}}{( score of selected response for ( Q 6 a + Q 7 a + … + Q 15 a ) )}$$

The weights and scores are substituted based on Table 5A to calculate or determine the second group score. The phrase or term ‘score selected response’ means the ‘score of/for response selected’, e.g. the phrase ‘score selected response for Q6b’ means the ‘score of/for response selected for Q6B as shown in the table applicable, for example Table 5A for Example 1 (whereas Table 5B for Examples 2, 2A, and 2B below).

Third Group score=(w16*r16)+(w17*r17)+(w18*r18)+(w19*r19)+(w20*r20)

When w16-w20 are substituted with their weights obtained from Table 6A,

Third Group score=(2*r16)+(1*r17)+(2*r18)+(1*r19)+(1*r20)

wherein r16-r20 are the responses, which are then substituted by scores r161, r162, etc. as selected and then values of r161, r162, etc. are substituted from Table 6A to obtain or determine the third group score.

The EDSS score group score and the age score are determined or calculated as shown in Table 7A.

Thereafter, the total score is calculated or determined as: Total score (TOTAL)=First group score+Second group score+Third group score+EDSS score+Age score.

Thereafter, the standardized total score may be optionally calculated or determined using the maximum first group score, the maximum second group score and the maximum third group score from Tables 4A, 5A and 6A, respectively and a maximum possible EDSS score and a maximum possible age score—each as determined or calculated from Table 7A:

$$Standardized total score = \frac{100 * Total}{44}$$

Based on the standardized total score the state of MS or the status of progression of MS from RRMS to SPMS is determined, e.g. if the standardized total score is below ‘50’ then MS is determined to be still in RRMS, if the standardized total score is between 50 and less than 70 then the determination is that the MS is progressing from RRMS to SPMS i.e. MS is in the ‘in-transition from RRMS to SPMS’ state, and optionally may provide a defined probability for example 60%-80% that the MS will be in SPMS state in a predetermined time, and if the standardized total score is 70 or above then there exists a high likelihood that MS has progressed from RRMS to SPMS i.e. a defined probability for example above 80%. A likelihood of above a certain percentage (e.g. 75% or above) may mean that the state of MS is SPMS.

### Example 2

In this example, the EDSS score and age score are determined together, and may be referred to as the ‘clinical score’. Table 4B, Table 5B and Table 6A (as provided for Example 1, hereinabove) provide the weights of the items Q1-Q20 and the scores of the corresponding responses to each of the items Q1-Q20 for this example.

TABLE 4B
First Group—items, item weights, corresponding predetermined
responses and response scores

| | | | | Maxi- |
|---|---|---|---|---|
| | | | | mum |
| | | | Corresponding | possible |
| | | Res- | predetermined responses | score |
| | Item | ponse | and scores (i.e. r11, r12, r21, | (for first |
| | weight | Score | .... r51, r52 as enlisted | Group = |
| Items | (wA) | (rA) | in Table 4 above) | 8) |
| Q1 | w1 = 3 | r1 | ‘Yes’ = 0; ‘No’ = 2 | 6 |
| Q2 | w2 = 2 | r2 | ‘1’ = 2; ‘2’ = 1; ‘3 or more’ = 0 | 4 |
| Q3 | w3 = 2 | r3 | ‘Full recovery (100%)’ = 0; ‘Nearly full | 4 |
| | | | recovery (75%)’ = 0; ‘Partial recovery | |
| | | | (50%)’ = 1; ‘Little recovery (25%)’ = 1; | |
| | | | ‘No recovery (0%)’ = 2 | |
| Q4 | w4 = 1 | r4 | ‘Yes’ = 0; ‘No’ = 0 | 0 |
| Q5 | w5 = 1 | r5 | ‘Yes’ = 0; ‘No’ = 0 | 0 |

TABLE 5B
Second Group—items, item weights, corresponding predetermined
responses and response scores.

| | | | | | Maximum |
|---|---|---|---|---|---|
| | | | | | possible score |
| | | | | Score of the | (for second |
| | | | | sub-items | group = 14, |
| | | | | (i.e. r6a1, r6a2, | based on the |
| | | | | r6b1, r6b2, | equation Ex1 |
| | | | | r6b3, r6c1 . . . | for |
| | Item | | | r15d3, r15d4 | calculating |
| | weight | | Weights | as enlisted | second group |
| | (for each | | of sub- | in Table 5 | score for this |
| Items | item) | Sub-item | item | above) | example) |
| Q6, Q9, | 1 | a | — | Yes = 1, No = 0 | |
| Q10, | (i.e. each | b if ‘Yes’ | 1 | Yes = 0, No = 1, | 1 |
| Q11, | of w6, | for a | | N/A = 0 | |
| Q12, | w9 to | c if ‘Yes’ | 1 | Intermittent = 0, | 3 |
| Q13, | w13, | for a | | Persistent = 3 | |
| Q15 | w15 = 1) | d if ‘Yes’ | 1 | Improving = 0, | 3 |
| | | for a | | Stable = 0, | |
| | | | | Worsening = 3, | |
| | | | | N/A = 0 | |
| Q7, Q8, | 2 | a | — | Yes = 1, No = 0 | |
| Q14 | (i.e. each | b if ‘Yes’ | 1 | Yes = 0, No = 1, | 2 |
| | of w7, | for a | | N/A = 0 | |
| | w8 and | c if ‘Yes’ | 1 | Intermittent = 0, | 6 |
| | w14 = 2) | for a | | Persistent = 3 | |
| | | d if ‘Yes’ | 1 | Improving = 0, | 6 |
| | | for a | | Stable = 0, | |
| | | | | Worsening = 3, | |
| | | | | N/A = 0 | |

Note:
Sub-item ‘a’ (i.e. Q6a, Q7a, Q8a, Q9a, Q10a, Q11a, Q12a, Q13a, Q14a, Q15a) in items Q6 to Q15 is used only as an indication of how many symptoms are present and therefore is not weighted.

Table 7B represents the weights and scores for the EDSS, the T25FW and age.

TABLE 7B
Clinical score - (EDSS and/or T25FW) and age

| | |
|---|---|
| Reweighted | Reweighted to be out of a total of 3 |
| EDSS group | score of ‘1’ if the EDSS score < 4 |
| score | score of ‘2’ if the EDSS score ≥ 4 and < 6 |
| RW_EDSS | score of ‘3’ if the EDSS score ≥ 6 |
| Reweighted | score of ‘1’ if T25FW < 6 seconds; |
| T25FW | score of ‘2’ if T25FW ≥ 6 and < 8 seconds |
| group score | score of ‘3’ if T25FW ≥ 8 seconds |
| RW_T25FW | |
| Age score | Score either 0 or 2, i.e. given a question weight of 2 |
| | $\{ \begin{matrix}age < 45 , & score = 0 \\ age \geq 45 , & score = 2\end{matrix}$ |

### Calculation of Total Score:

The item score for each item Q1-Q20, when all the items Q1 to Q20 are included in the first, the second and the third groups, is determined or calculated by multiplying the item weight with the corresponding response selected for that item. In case of items of the second group, i.e. items Q6 to Q15, the sub-item scores are calculated to indicate the item score.

$$First Group score = \frac{( w 1 * r 1 ) + ( w 2 * r 2 ) + ( w 3 * r 3 ) + ( w 4 * r 4 ) + ( w 5 * r 5 )}{maximum possible group score for the first group}$$

When w1-w5 are substituted with their weights obtained from Table 4B, and when the maximum possible first group score from Table 4B is used:

$$First Group score = \frac{( 3 * r 1 ) + ( 2 * r 2 ) + ( 2 * r 3 ) + ( 1 * r 4 ) + ( 1 * r 5 )}{8}$$

wherein r1-r5 are the responses, which are then substituted by scores r11, r12, etc. as selected and then values of r11, r12, etc. are substituted from Table 4B to obtain or determine the first group score. For example if the response for the item Q1 is chosen or selected as ‘Yes’, the score r1 is substituted by r11 (as shown in Table 4) and thereafter the score r11 is substituted by value or score zero, ‘0’ (as shown in Table 4B), similarly for example if the response for the item Q3 is chosen or selected as ‘No recovery’, the score r3 is substituted by r35 (as shown in Table 4) and thereafter the score r35 is substituted by value or score 2 (as shown in Table 4B).

Second group score is determined or calculated in the same way as described hereinabove for example 1, albeit by using weights and scores from Table 5B.

$$Third Group score = \frac{( w 16 * r 16 ) + ( w 17 * r 17 ) + ( w 18 * r 18 ) + ( w 19 * r 19 ) + ( w 20 * r 20 )}{maximum possible group score for the third group}$$

When w16-w20 are substituted with their weights obtained from Table 6A, and when the maximum possible first group score from Table 6A is used:

$$Third Group score = \frac{( 2 * r 16 ) + ( 1 * r 17 ) + ( 2 * r 18 ) + ( 1 * r 19 ) + ( 1 * r 20 )}{8}$$

wherein r16-r20 are the responses, which are then substituted by scores r161, r162, etc. as selected and then values of r161, r162, etc. are substituted from Table 6A to obtain or determine the third group score.

As stated hereinabove, in this example the EDSS score and the age score are referred to as the ‘clinical score’. In other words, determining the EDSS score and determining the age score can be together referred to as determining the clinical score. The clinical score, i.e. the EDSS score group score and the age score are determined or calculated as follows using Table 7B.

Clinical score=EDSS group score+Age score={RW_EDSS+age score}/5

Alternatively, if the EDSS score is unavailable, the T25FW may be used in place of the EDSS score as follows:

Clinical score=T25FW group score+Age score={RW_T25FW)+age score}/5

Alternatively, if both EDSS and T25FW are missing then:

Clinical score=(age score)/2

Thereafter, the total score is calculated or determined as: Total score (TOTAL)=First group score+Second group score+Third group score+EDSS score+Age score.

In other words, the total score is calculated or determined as: Total score (TOTAL)=First group score+Second group score+Third group score+Clinical score

Thereafter, the standardized total score may be optionally calculated or determined:

$$Standardized total score = \frac{100 * Total}{44}$$

The value of ‘4’ is used to represent the four group scores i.e. the score of the first group, the score of the second group, the score of the third group, and the clinical score.

This example (Example 2) uses two different thresholds—a first threshold value and a second threshold value. For example, the first threshold value may be about ‘46’ whereas the second threshold value may be about ‘58’. Therefore, based on the standardized total score the state of MS i.e. the status of progression of MS from RRMS to SPMS is determined, e.g. if the standardized total score is below the first threshold value, e.g. below about ‘46’, then the state of MS is determined, by the one or more processors, to be in RRMS state. If the standardized total score is equal to or above the first threshold value but below the second threshold value, e.g. equal to or more than about ‘46’ and less than about ‘58’, then the state of MS is determined, by the one or more processors, to be in transition from RRMS to SPMS i.e. MS is in the ‘in-transition from RRMS to SPMS’ state. If the standardized total score is equal to or above the second threshold, e.g. about ‘58’ or above, then the state of MS is determined, by the one or more processors, to be SPMS state.

In another embodiment, i.e. say Example 2A, of Example 2, instead of using Table 7B, Table 7C, as presented hereinabove, may be used, while keeping all other aspects the same. So, in Example 2A, the first group comprises all Q1-Q5, the second group comprises all Q6-Q15 and the third group comprises all Q16-Q20. Also, in Example 2A, the EDSS score and age score are determined together by using Table 7C and may be referred to as the ‘clinical score’. Furthermore, Table 4B, Table 5B and Table 6A (as provided for Example 1, hereinabove) provide the weights of the items Q1-Q20 and the scores of the corresponding responses to each of the items Q1-Q20 for this example. The item scores, the first group score, the second group score, the third group score, the clinical score (using the EDSS group score or T25FW group score score), the total score (TOTAL) and the standardized total score are calculated or determined as explained for Example 2 hereinabove.

Table 7C represents the weights and scores for the EDSS, the T25FW and age.

TABLE 7C
Clinical score - (EDSS and/or T25FW) and age

| | |
|---|---|
| Reweighted | Reweighted to be out of a total of 3 |
| EDSS group | score of ‘1’ if the EDSS score < 3 |
| score | score of ‘2’ if the EDSS score ≥ 3 and < 6 |
| RW_EDSS | score of ‘3’ if the EDSS score ≥ 6 |
| Reweighted | score of ‘1’ if T25FW < 6 seconds; |
| T25FW | score of ‘2’ if T25FW ≥ 6 and < 8 seconds |
| group score | score of ‘3’ if T25FW ≥ 8 seconds |
| RW_T25FW | |
| Age score | Score either 0 or 2, i.e. given a question weight of 2 |
| | $\{ \begin{matrix}age < 45 , & score = 0 \\ age \geq 45 , & score = 2\end{matrix}$ |

In the Example 2A, two different thresholds or cut-off are used—a first threshold value or cut-off value representing a cut-off for RRMS state and a second threshold value or cut-off value representing a cut-off for SPMS state, when the clinical score used in determination of total score is calculated using the EDSS score or the T25FW score. Thus in this case if the standardized total score is below the first threshold value, then the one or more processors determines the state of MS as RRMS state, whereas the if the standardized total score is equal to or above the second threshold value, then the one or more processors determines the state of MS as SPMS state, however if the standardized total score is equal to or above the first threshold value but lower than the second threshold value, then the one or more processors determines the state of MS as ‘in-transition from RRMS to SPMS’ state. For example, the first threshold value may be about ‘51.8’ whereas the second threshold value may be about ‘58.85’. The ‘in-transition from RRMS to SPMS’ state, generally, including the examples and embodiments explained in this disclosure, indicates that the MS in patient has developed symptoms/effects of early SPMS state.

In another embodiment, i.e. say Example 2B, of Example 2, which is same as Example 2A, except that clinical score is determined without the EDSS score or the T25FW score, two different thresholds or cut-off are used—a first threshold value or cut-off value representing a cut-off for RRMS state and a second threshold value or cut-off value representing a cut-off for SPMS state, when the clinical score used in determination of total score is calculated without using the EDSS score or the T25FW score. Thus in this case if the standardized total score is below the first threshold value, then the one or more processors determines the state of MS as RRMS state, whereas if the standardized total score is equal to or above the second threshold value, then the one or more processors determines the state of MS as SPMS state, however if the standardized total score is equal to or above the first threshold value but below or lower than the second threshold value, then the one or more processors determines the state of MS as ‘in-transition from RRMS to SPMS’ state. For example, the first threshold value may be about ‘46.3’ whereas the second threshold value may be about ‘57.8’.

The above-stated values of the first and the second threshold for each of the examples—Example 2, Example 2A and Example 2B—are for comparing with the standardized total score. However, it may be appreciated that for each of the examples—Example 2, Example 2A and Example 2B, equivalent or comparable values for the first and the second threshold may be determined or fixed or calculated for comparing with total score, i.e. when the total score has not been standardized.

Examples 2, 2A, and 2B—first (i.e. Example 2) with Table 7B and second (i.e. Examples 2A and 2B) with Table 7C, provides example of how each of the first group score, the second group score and the third group score is determined or calculated by using weighted averaging, as explained hereinabove.

### Example 3

The state of MS, i.e. either RRMS or SPMS is determined according to any one of the examples—i.e. Example 1 or Example 2.

Thereafter, if the state of MS is determined to be RRMS, the patient is administered fingolimod, for example a pharmaceutically acceptable salt such as fingolimod hydrochloride or co-crystal thereof. A daily dosage of 0.5 mg of fingolimod is administered, based on fingolimod in form of the free base. The daily dosage may be administered orally.

However, if the state of MS is determined to be SPMS, the patient is administered siponimod. The siponimod is administered in form of siponimod hemifumarate or in form of a co-crystal of siponimod and fumaric acid. A daily dosage of 2.0 mg of siponimod is administered, based on siponimod in form of the free base. The daily dosage may be administered orally.

Furthermore, when the state of MS comprises the third state i.e. in-transition from RRMS to SPMS state, and if the state of MS is determined to be in the third state, the patient may be administered siponimod. The siponimod may be administered in form of siponimod hemifumarate or in form of a co-crystal of siponimod and fumaric acid. A daily dosage of 2.0 mg of siponimod may be administered, based on siponimod in form of the free base. The daily dosage may be administered orally.

Computer networks suitable for use with the embodiments, as well as the examples—Example 1, 2 and 3, described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The technique for determining a state of Multiple Sclerosis in a patient can be a component of a single system, multiple systems, and/or geographically separate systems. The technique for determining a state of Multiple Sclerosis in a patient can also be a subcomponent or subsystem of a single system, multiple systems, and/or geographically separate systems. The components of technique for determining a state of Multiple Sclerosis in a patient can be coupled to one or more other components (not shown) of a host system or a system coupled to the host system.

One or more components of the technique for determining a state of Multiple Sclerosis in a patient and/or a corresponding interface, system or application to which the technique for determining a state of Multiple Sclerosis in a patient is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the technique for determining a state of Multiple Sclerosis in a patient can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the technique for determining a state of Multiple Sclerosis in a patient and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the technique for determining a state of Multiple Sclerosis in a patient and corresponding systems and methods include: microcontrollers with memory (such as electronically erasable programmable read only memory (EEPROM)), embedded microprocessors, firmware, software, etc. Furthermore, aspects of the technique for determining a state of Multiple Sclerosis in a patient and corresponding systems and methods may be embodied in microprocessors having software-based circuit emulation, discrete logic (sequential and combinatorial), custom devices, fuzzy (neural) logic, quantum devices, and hybrids of any of the above device types. Of course, the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

### Methodology

Regression analysis on data from a real-world observational study, including 250 neurologists (actively managing MS) and 3294 MS patients in the United States, identified the variables that were significant drivers of the difference between RRMS and SPMS patients.

Firstly, unadjusted/bivariate analyses were performed to identify variables that could potentially differentiate RRMS from SPMS. Fisher's exact test and Mann-Whitney test were used for categorical and continuous outcomes respectively, to determine unadjusted differences in the two groups—i.e. RRMS patients and SPMS patients.

Based on the results from bivariate analyses, clinical experience and the need to include relevant variables that cover all aspects of disease, the following key variables were selected for regression analysis—Demographics, Employment status, Number of T2 lesions, Requirement of help for activities of daily living, Presence of motor, parathesia/sensory, ataxia/co-ordination, micturition/bladder, mood/depression and concentration/cognition symptoms.

Thereafter, multivariate analysis was performed. Lasso penalised logistic regression was used to determine variables associated with early-RRMS or early-SPMS. Bootstrap-based 95% confidence intervals were produced. Regression coefficients were used to generate the predicted probability of being early-SPMS for the late-RRMS patients. In the multivariate analysis, positive non-zero coefficients (which indicates higher likelihood of being early-SPMS) were obtained for several variables.

The determination of variables (i.e. including queries, EDSS score, T25FW score, age), respective weights and scores, and formulae for calculating or determining item scores, group scores, EDSS/T25FW score, total score as applicable for embodiments and examples described herein was developed using input from three approaches: A. Regression analysis—the multiple logistic regression on real-world data as described hereinabove, B. Qualitative interviews—with patients and physicians, and C. physician completion of ranking/weighting exercise for each variable.

### A. Regression Analysis

Multiple logistic regression conducted on the observational study variables (n=2791) helped identify how strongly each variable contributed to SPMS diagnosis. Multiple logistic regression analyses identified Expanded Disability Status Scale score (odds ratio, 1.79; p<0.0001), age (1.04; p<0.0001) and MS disease activity (1.68; p<0.05) as the most significant physician-reported predictors of progression to SPMS (see Table A below). Patient age (1.05; p<0.0001), mobility (4.46, p<0.0001) and self-care (2.39; p<0.0001) were identified as the strongest patient-reported predictors of progression to SPMS (see Table B below).

TABLE A

| Physician completed patient | Odds ratio | p va1ue |
|---|---|---|
| record form variables | (95% CI) | for z-test |
| Patient age | 1.04 (1.02; 1.05) | <0.0001 |
| Time since MS diagnosis | 1.03 (1.00; 1.07) | 0.062 |
| MS disease activity* | 1.68 (1.02; 2.77) | 0.041 |
| MRI scan, No T2 activity | 0.81 (0.43; 1.51) | 0.508 |
| MRI scan, T2 activity | 1.07 (0.57; 2.01) | 0.826 |
| Current EDSS symptoms | 1.79 (1.59; 2.02) | <0.0001 |
| Motor/ambulatory symptoms | 1.03 (0.65; 1.64) | 0.903 |
| Co-ordination/balance symptoms | 1.34 (0.90; 1.97) | 0.145 |
| Bladder/bowel symptoms | 1.05 (0.65; 1.71) | 0.826 |
| Speech symptoms | 1.15 (0.70; 1.90) | 0.584 |
| Cognitive symptoms | 0.69 (0.45; 1.05) | 0.082 |

*Physician reported MS disease activity based on symptoms and recent relapse, and ranging from ‘no activity to high activity’;
an odds ratio >1 imp1ies a higher risk of SPMS;
CI = confidence intervals;
EDSS = Expanded Disability Status Scale;
MIR = magnetic resonance imaging;
MS = multiple sclerosis

TABLE B

| Patient self-completion | Odds ratio | p va1ue |
|---|---|---|
| form variables | (95% CI) | for z-test |
| Patient age | 1.04 (1.02; 1.07) | <0.0001 |
| Mobility | 4.46 (2.05; 9.70) | <0.0001 |
| Self-care | 2.39 (1.52; 3.76) | <0.0001 |
| Usual activities | 1.30 (0.67; 2.55) | 0.434 |
| Pain/discomfort | 0.86 (0.47; 1.58) | 0.636 |
| Fatigue | 0.99 (0.37; 2.59) | 0.978 |
| Cognition | 0.88 (0.47; 1.64) | 0.698 |
| Vision disturbance | 0.96 (0.52; 1.74) | 0.888 |
| Bowel/bladder | 1.49 (0.84; 2.66) | 0.175 |

an odds ratio >1 imp1ies a higher risk of SPMS;
CI = confidence intervals;
EDSS = Expanded Disability Status Scale;
MRI = magnetic resonance imaging;
MS = multiple sclerosis

### B. Qualitative Interviews

Interviews were conducted with patients and physicians to obtain qualitative insight into importance of each variable in the diagnosis of SPMS. An overview of the variables of high, moderate and low importance is presented in Table C, Table D, and Table E. Ranking out of 26 variables was included. Lower ranking indicates greater importance.

TABLE C
Variables of high importance in progression from RRMS to SPMS

| | Average | | |
|---|---|---|---|
| | assigned | Average | |
| | rank* | weighting | Reasons provided by |
| | (range: | (range: | physicians for |
| | highest- | lowest- | ranking and |
| Variables | lowest) | highest) | weighting |
| Stability- | 5 (1-11.5) | 11% (1-33%) | Worsening of symptoms is an |
| worsening | | | important indication of SPMS |
| of symptoms | | | progression |
| Intermittent | 7 (2-15.5) | 7% (0-17%) | Intermittent symptoms can |
| or persistent | | | indicate a relapse, whereas |
| symptoms | | | persistent symptoms are likely |
| | | | to indicate progression |
| Presence of | 8 (1.5-15) | 6% (0-10%) | Impact the daily lives of |
| ambulatory | | | patients and tend to be |
| symptoms | | | experienced in patients that are |
| | | | progressing to SPMS |
| Impact on | 11 (3.5-20) | 6% (0-10%) | Decline in mobility is an |
| mobility | | | important predictor of SPMS |
| | | | progression |
| Presence of | 9 (5-19) | 6% (1-10%) | Impact patients' lives and |
| cognitive | | | indicate gradual worsening, |
| symptoms | | | particularly when experienced |
| | | | persistently |
| Relapses | 11 (2-23.5) | 6% (0-10%) | Experience of symptoms |
| in the past | | | without the presence of a |
| 6 months | | | relapse can indicate SPMS |
| | | | progression |
| EDSS score | 10 (1.5-26) | 5% (0-10%) | Objective indication of |
| | | | progression comprising impact |
| | | | on ambulation and mobility |
| Time since | 10 (1-25) | 5% (0-15%) | Progression to SPMS is |
| diagnosis | | | unlikely in patients with a |
| | | | recent diagnosis |

*Ranking out of 26 variables

TABLE D
Variables of moderate importance in progression from RRMS to SPMS

| | Average | | |
|---|---|---|---|
| | assigned | Average | |
| | rank* | weighting | |
| | (range: | (range: | |
| | highest- | lowest- | Reasons provided by physicians |
| Variables | lowest) | highest) | for ranking and weighting |
| Signs of new | 14 (1-23.5) | 6% (0-20%) | Objective measurement |
| activity based | | | providing evidence of activity, |
| on MRI scans | | | though not always related to |
| | | | progression |
| Recovery from | 13 (3-22) | 5% (0-12%) | Failure to recover from a relapse |
| most recent | | | can be an indicator of |
| relapse | | | persistence and therefore |
| | | | progression to SPMS |
| Impact | 13 (7-25) | 5% (1-10%) | Can be an early signal of |
| on daily | | | progression to SPMS but may |
| activities | | | not be specific enough to be an |
| | | | important indicator |
| Presence | 11 (4-17.5) | 5% (0-10%) | Might be linked to both relapse |
| of motor | | | and progression, but considered |
| symptoms | | | important as it seriously impacts |
| | | | daily life |
| Presence of | 12 (3.5-20) | 5% (0-10%) | Ranking and weighting varied |
| coordination | | | among the physicians; some felt |
| and balance | | | that alone it was not specific |
| symptoms | | | enough to identify a progression |
| | | | to SPMS |
| Number of | 11 (1-24.5) | 4% (0-15%) | Majority of physicians felt that |
| relapses in the | | | high number of relapses in the |
| past 6 months | | | past 6 months can be an |
| | | | indication of SPMS but not to be |
| | | | used alone |
| Symptoms | 15 (1-24) | 4% (0-14%) | Symptom activity can identify |
| during | | | when a patient stops having |
| relapse | | | relapses and starts progressing |
| Presence of | 17 (7-26) | 3% (0-10%) | Important due to the impact on |
| speech | | | patients' lives, but not a clear |
| symptoms | | | indication of SPMS |

*Ranking out of 26 variables

TABLE E
Variables of low importance in progression from RRMS to SPMS

| | Average | | |
|---|---|---|---|
| | assigned | Average | |
| | rank* | weighting | |
| | (range: | (range: | |
| | highest- | lowest- | Reasons provided by physicians |
| Variables | lowest) | highest) | for ranking and weighting |
| Presence | 19 (6-24) | 2% (0-10%) | Hard to measure, fairly |
| of fatigue | | | subjective and does |
| | | | not necessarily indicate |
| | | | progression |
| Impact | 18 (8-25) | 2% (0-10%) | Not a specific indication of |
| on hobbies | | | progression, and also may not |
| and leisure | | | be relevant to all MS patients |
| time | | | |
| Presence | 16 (8-24) | 2% (0-5%) | Can occur at any stage |
| of visual | | | of MS, and therefore |
| symptoms | | | are not an indicator of |
| | | | progression to SPMS |
| Presence | 16 (12-20) | 2% (0-5%) | General symptoms of MS that |
| of bladder | | | cannot be considered as an |
| and bowel | | | important indicator for |
| symptoms | | | progression to SPMS |
| Impact on | 16 (8-26) | 2% (0-5%) | Impacts daily lives of |
| self-care | | | patients but not specific |
| | | | enough to indicate |
| | | | progression to SPMS |
| Impact | 16 (9-24) | 2% (0-5%) | A subjective variable dependent |
| on paid and | | | on the patient's type of |
| unpaid work | | | employment, type of insurance |
| | | | (for US patients) and severity |
| | | | of symptoms |
| MRI in past 6 | 18 (4-26) | 1% (0-5%) | An MRI being performed is not |
| months | | | itself indicative of progression |
| Presence | 21 (16.5-26) | 1% (0-5%) | Not an important indication of |
| of pain | | | SPMS, particularly as the |
| symptoms | | | current variable does not |
| | | | specify persistent pain |
| Age | 18 (1.5-26) | 1% (0-4%) | Though older patients are more |
| | | | likely to progress, SPMS can |
| | | | occur at any age and therefore |
| | | | age is not a significant factor |
| Presence | 16 (12-21) | 1% (0-2%) | Common in MS |
| of sensory | | | patients and very |
| symptoms | | | subjective, therefore not an |
| | | | important indication of SPMS |

*Ranking out of 26 variables

### C. Physician Ranking and Weighting Exercise

Physician (n=8, 4 in US i.e. United States of America and 4 in Germany) ranking and weighting exercise were used to elicit a ranking position and individual weight contribution for each variable and to explore the level of concordance between physicians. Overall, the level of concordance for the variable ranking among eight physicians was 0.278 (p=0.0004) which indicates a significant, but low to moderate level of agreement. There was a greater level of agreement within countries (US: 0.522, Germany: 0.385; p<0.05), but lesser between countries. Level of agreement for the variable weighting was significant but relatively weak (intraclass correlation coefficient=0.12; p=0.0025).

### Ranking and Weighing of Variables

The variables were identified based on ranks (see Table F) and weights (see Table G). Both tables—Table F and Table G show top ten variables only.

TABLE F
Variable ranked in top ten

| Variables (Top 10)* | Average rank |
|---|---|
| Improving, stable or worsening | 5.1 |
| Intermittent or persistent | 6.9 |
| Ambulatory symptoms | 8.3 |
| Cognitive symptoms | 8.9 |
| EDSS score | 10.1 |
| Time since diagnosis | 10.4 |
| Mobility | 10.6 |
| Number of relapses | 10.8 |
| Motor symptoms | 11.1 |
| Any relapses | 11.2 |

*Top 10 not listed in order. Lower ranking indicates greater importance.

TABLE G
Variable weighted in top ten

| Variables (Top 10)* | Average weight |
|---|---|
| Improving, stable or worsening | 9.9 |
| Intermittent or persistent | 6.4 |
| New MRI activity | 6.2 |
| Cognitive symptoms | 5.9 |
| Mobility | 5.5 |
| Ambulatory symptoms | 5.2 |
| EDSS score | 5.2 |
| Any relapses | 5.1 |
| Co-ordination symptoms | 4.8 |
| Daily activities | 4.7 |

*Top 10 not listed in order. Higher weighting indicates greater importance.

Based on the results from all the three analyses as mentioned hereinabove i.e. A. Regression analysis, B. Qualitative interviews and associated analysis, and C. Physician ranking and weighting exercise and concordance levels among physician ranking and weighting—items were assigned weights. For example, as depicted in Examples 1, 2 and 3 herein, in as items were assigned weights as follow: ‘3’ for variables that were found to be important, ‘2’ for variables that were found to be moderately important, and ‘1’ for variables that were found to be less important.

While the present technique has been described in detail with reference to certain embodiments, it should be appreciated that the present technique is not limited to those precise embodiments. Rather, in view of the present disclosure which describes exemplary modes for practicing the invention, many modifications and variations would present themselves, to those skilled in the art without departing from the scope of the invention indicated by the following claims. All changes, modifications, and variations coming within the meaning and range of equivalency of the claims are to be considered within their scope.

1. A system for determining a state of Multiple Sclerosis (MS) in a MS patient, the system comprising:
a display (10) configured to output a graphical user interface (12) comprising fields for displaying patient data queries and age query for inputting of responses to the patient data queries and the age query,
wherein the patient data queries comprise a plurality of items grouped into a first group, a second group and a third group, wherein the first group comprises at least one item relating to relapse and recovery of the patient in a first predetermined period, the second group comprises at least one item relating to symptoms experienced by the patient in a second predetermined period and the third group comprises at least one item relating to impacts experienced by the patient in a third predetermined period,
wherein each item of the plurality of items has an assigned predetermined weight and comprises a plurality of corresponding predetermined responses, and wherein each response of the corresponding predetermined responses has an assigned predetermined score and is indicative of a distinct information pertaining to the corresponding item;
a user interface (20) for inputting of responses to the patient data queries and for inputting of an age of the patient as a response to the age query, wherein at least one response from the plurality of corresponding predetermined responses for each of the plurality of items displayed in the graphical user interface is selectable for inputting of the responses to the patient data queries;
one or more processors (30) configured to receive the responses to the patient data queries and the age query and to:
determine an item score for each of the plurality of items based on the weight of the item and the score of the selected response for the item;
determine a first group score, a second group score and a third group score based on the item scores of the items of the first group, the second group and the third group, respectively;
allocate an inputted age of the patient to one of a plurality of predetermined age groups, wherein each of the predetermined age groups has an assigned predetermined score;
determine an age score based on the score of the allocated age group and a predetermined weight assigned for the age query;
generate a total score based on the first group score, the second group score, the third group score and the age score;
determine, based on the total score, the state of MS in the patient, wherein the state of MS comprises either a first state or a second state; and
an output means (40) configured to indicate the state of MS so determined by the one or more processors.

2. The system according to claim 1, wherein the first state comprises a Relapsing-Remitting Multiple Sclerosis (RRMS) state.

3. The system according to claim 1 or 2, wherein the second state comprises a Secondary Progressive Multiple Sclerosis (SPMS) state.

4. The system according to any one of claims 1 to 3, wherein the state of MS comprises a third state and wherein the third state is in-transition from RRMS to SPMS state, when the first state is RRMS and the second state is SPMS.

5. The system according to any one of claims 1 to 4, wherein the one or more processors (30) is configured to generate the total score by adding the first group score, the second group score, the third group score, and the age score.

6. The system according to any one of claims 1 to 4, wherein the graphical user interface (12) further comprises at least one of a field for Expanded Disability Status Scale (EDSS) query and a field for Timed 25-Foot Walk (T25FW) query for inputting of responses to the at least one of the EDSS, query and the T25FW query;
wherein the user interface (20) is configured for inputting of an EDSS score of the patient as a response to the EDSS query and/or for inputting of an T25FW score of the patient as a response to the T25FW query; and
wherein the one or more processors (30) is configured: to receive the responses to the EDSS query and/or the T25FW query, to determine an EDSS group score based on an inputted EDSS score and a predetermined weight assigned for the EDSS query and/or to determine an T25FW group score based on an inputted T25FW score and a predetermined weight assigned for the T25FW query, to generate the total score based on the first group score, the second group score, the third group score, the age score and at least one of the EDSS group score and the T25FW group score.

7. The system according to claim 6, wherein the one or more processors (30) is configured to generate the total score by adding the first group score, the second group score, the third group score, and the age score and at least one of the EDSS group score and the T25FW group score.

8. The system according to claim 6 or 7, wherein the one or more processors (30) is configured to determine the EDSS group score by:
acquiring the EDSS score for the patient,
generating a weighted EDSS score from the acquired EDSS score as a fraction of the maximum possible score of the Expanded Disability Status Scale, and
generating a reweighted EDSS score by multiplying the weighted EDSS score and the predetermined weight for the EDSS score.

9. The system according to any one of claims 1 to 8, wherein the one or more processors (30) is configured to determine the item score by multiplying the weight of the item and the score of the selected response for the item.

10. The system according to any one of claims 1 to 9, wherein the one or more processors (30) is configured to determine the first group score, the second group score and the third group score by adding the item scores of the items comprised in the first group, the second group and the third group, respectively.

11. The system according to any one of claims 1 to 9, wherein the one or more processors (30) is configured to determine the first group score, the second group score and the third group score by weighted average of the item scores of the items comprised in the first group, the second group and the third group, respectively.

12. The system according to any one of claims 1 to 11, wherein the one or more processors (30) is configured to determine the age score by multiplying the score of the allocated age group and the predetermined weight assigned for the age.

13. The system according to any one of claims 1 to 12, wherein the first predetermined period, the second predetermined period and the third predetermined period are same.

14. The system according to any one of claims 1 to 13, wherein each of the first predetermined period, the second predetermined period and the third predetermined period is 6 months from a predetermined date.

15. The system according to any one of claims 1 to 14, wherein the items of the first group are selected from:
an item indicating if the patient has experienced any relapses,
an item indicating a number of relapses the patient has experienced,
an item indicating an extent of recovery of the patient from a last relapse,
an item indicating if a Magnetic Resonance Imaging (MRI) has been performed on the patient, and
an item indicating if the performed MRI showed new signs of activity related to MS.

16. The system according to claim 15, wherein the first group comprises all the items of claim 15.

17. The system according to any one of claims 1 to 16, wherein the items of the second group are selected from:
an item indicating visual symptoms related to MS,
an item indicating motor symptoms related to MS,
an item indicating ambulatory symptoms related to MS,
an item indicating coordination and balance symptoms related to MS,
an item indicating pain experienced due to MS,
an item indicating sensory symptoms related to MS,
an item indicating bladder and bowel symptoms related to MS,
an item indicating speech symptoms related to MS,
an item indicating cognitive symptoms related to MS, and
an item indicating fatigue symptoms related to MS.

18. The system according to claim 17, wherein the second group comprises all the items of claim 17.

19. The system according to any one of claims 1 to 18, wherein the items of the third group are selected from:
an item indicating impact on mobility of the patient due to MS,
an item indicating impact on self-care of the patient due to MS,
an item indicating impact on daily activities of the patient due to MS,
an item indicating impact on hobbies and leisure time of the patient due to MS, and
an item indicating impact on paid and unpaid work of the patient due to MS.

20. The system according to claim 19, wherein the third group comprises all the items of claim 19.

21. The system according to any one of claims 1 to 20, wherein the output means (40) is configured to indicate the state of MS in the MS patient by one of a visual output, an audio output, a tactile output, and a combination thereof.

22. A method for determining a state of Multiple Sclerosis (MS) in a MS patient, the method comprising:
causing (110), by one or more processors (40), a graphical user interface (12) comprising fields for patient data queries and age query to be output on a display for inputting of responses to the patient data queries and the age query,
wherein the patient data queries comprise a plurality of items grouped into a first group, a second group and a third group, wherein the first group comprises at least one item relating to relapse and recovery of the patient in a first predetermined period, the second group comprises at least one item relating to symptoms experienced by the patient in a second predetermined period and the third group comprises at least one item relating to impacts experienced by the patient in a third predetermined period,
wherein each item of the plurality of items has an assigned predetermined weight and comprises a plurality of corresponding predetermined responses, and wherein each response of the corresponding predetermined responses has an assigned predetermined score and is indicative of a distinct information pertaining to the corresponding item;
inputting (120) of responses to the patient data queries by selecting at least one response from the plurality of corresponding predetermined responses for each of the plurality of items displayed in the graphical user interface (12);
determining (130), by the one or more processors (30), an item score for each of the plurality of items based on the weight of the item and the score of the selected response for the item;
determining (140), by the one or more processors (30), a first group score, a second group score and a third group score based on the item scores of the items of the first group, the second group and the third group, respectively;
inputting (170) of an age of the patient as a response to the age query in the graphical user interface (12), wherein the age of the patient is allocated to one of a plurality of predetermined age groups, wherein each of the predetermined age groups has an assigned predetermined score;
determining (180), by the one or more processors (30), an age score based on the score of the allocated age group and a predetermined weight assigned for the age query;
generating (190), by the one or more processors, a total score based on the first group score, the second group score, the third group score and the age score;
determining (200), by the one or more processors, the state of MS in the patient based on the total score, wherein the state of MS comprises either a first state or a second state; and
outputting (210), by an output means, the state of MS so determined.

23. The method according to claim 22, wherein the first state comprises a Relapsing-Remitting Multiple Sclerosis (RRMS) state.

24. The method according to claim 22 or 23, wherein the second state comprises a Secondary Progressive Multiple Sclerosis (SPMS) state.

25. The method according to any one of claims 22 to 24, wherein in determining (200), by the one or more processors, the state of MS in the patient based on the total score, the state of MS comprises a third state, wherein the third state is in-transition from RRMS to SPMS state, when the first state is RRMS and the second state is SPMS.

26. The method according to any one of claims 22 to 25, wherein generating (190) the total score comprises adding the first group score, the second group score, the third group score and the age score.

27. The method according to any one of claims 22 to 25, wherein the graphical user interface (12), caused by the one or more processors (40) to be output on the display, further comprises at least one of a field for Expanded Disability Status Scale (EDSS) query and a field for Timed 25-Foot Walk (T25FW) query for inputting of responses to the at least one of the EDSS query and the T25FW query; and
wherein the method further comprises:
inputting (150) of at least one of an EDSS score of the patient as a response to the EDSS query in the graphical user interface (12) and an T25FW score of the patient as a response to the T25FW query in the graphical user interface (12);
determining (160), by the one or more processors (30), an EDSS group score based on the inputted EDSS score and a predetermined weight assigned for the EDSS query and/or determining (160), by the one or more processors (30), a T25FW group score based on the inputted T25FW score and a predetermined weight assigned for the T25FW query; and
wherein in generating (190) the total score, the total is generated based on the first group score, the second group score, the third group score and the age score and at least one of the EDSS group score and the T25FW group score.

28. The method according to claim 27, wherein generating (190) the total score comprises adding the first group score, the second group score, the third group score and the age score and at least one of the EDSS group score and the T25FW group score.

29. The method according to claim 27 or 28, wherein determining (160) the EDSS group score comprises:
acquiring the EDSS score for the patient,
generating a weighted EDSS score from the acquired EDSS score as a fraction of the maximum possible score of the Expanded Disability Status Scale, and
generating a reweighted EDSS score by multiplying the weighted EDSS score and the predetermined weight for the EDSS score.

30. The method according to any one of claims 22 to 29, wherein determining (130) the item score comprises multiplying the weight of the item and the score of the selected response for the item.

31. The method according to any one of claims 22 to 30, wherein determining (140) the first group score, the second group score and the third group score comprises adding the item scores of the items comprised in the first group, the second group and the third group, respectively.

32. The method according to any one of claims 22 to 30, wherein determining (140) the first group score, the second group score and the third group score comprises determining a weighted average of the item scores of the items comprised in the first group, the second group and the third group, respectively.

33. The method according to any one of claims 22 to 32, wherein determining (180) the age score comprises multiplying the score of the allocated age group and the predetermined weight assigned for the age.

34. The method according to any one of claims 22 to 33, wherein the first predetermined period, the second predetermined period and the third predetermined period are same.

35. The method according to any one of claims 22 to 34, wherein each of the first predetermined period, the second predetermined period and the third predetermined period is six months from a predetermined date.

36. The method according to any one of claims 22 to 35, wherein the items of the first group are selected from:
an item indicating if the patient has experienced any relapses,
an item indicating a number of relapses the patient has experienced,
an item indicating an extent of recovery of the patient from a last relapse,
an item indicating if a Magnetic Resonance Imaging, MRI, has been performed on the patient, and
an item indicating if the performed MRI showed new signs of activity related to MS.

37. The method according to claim 36, wherein the first group comprises all the items of claim 36.

38. The method according to any one of claims 22 to 37, wherein the items of the second group are selected from:
an item indicating visual symptoms related to MS,
an item indicating motor symptoms related to MS,
an item indicating ambulatory symptoms related to MS,
an item indicating coordination and balance symptoms related to MS,
an item indicating pain experienced due to MS,
an item indicating sensory symptoms related to MS,
an item indicating bladder and bowel symptoms related to MS,
an item indicating speech symptoms related to MS,
an item indicating cognitive symptoms related to MS, and
an item indicating fatigue symptoms related to MS.

39. The method according to claim 38, wherein the second group comprises all the items of claim 38.

40. The method according to any one of claims 22 to 39, wherein the items of the third group are selected from:
an item indicating impact on mobility of the patient due to MS,
an item indicating impact on self-care of the patient due to MS,
an item indicating impact on daily activities of the patient due to MS,
an item indicating impact on hobbies and leisure time of the patient due to MS, and
an item indicating impact on paid and unpaid work of the patient due to MS.

41. The method according to claim 40, wherein the third group comprises all the items of claim 40.

42. The method according to any one of claims 22 to 41, wherein outputting (210) the state of MS comprises providing one of a visual output, an audio output, a tactile output, and a combination thereof indicating the state of MS.

43. One or more non-transitory computer-readable media storing computer-executable instructions that, when executed by one or more processors, cause the one or more processors to perform a method according to any one of the claims 22 to 42.

44. A method for treating multiple sclerosis (MS) in a patient in need thereof, the method comprising:
determining in the patient the state of MS according to any one of claims 22 to 42; and
administering to the patient an MS therapeutic based on the state.

45. The method according to claim 44, wherein the MS therapeutic is an sphingosine-1-phosphate (S1P) receptor modulator which can be administered orally.

46. The method according to claim 45, wherein the S1P receptor modulator is fingolimod or a pharmaceutically acceptable salt or co-crystal thereof.

47. The method according to claim 46, wherein the fingolimod salt is fingolimod hydrochloride.

48. The method according to claim 46 or 47, wherein the patient is administered fingolimod if the state of MS in the patient is the first state, wherein the first state is RRMS state.

49. The method according to any one of claims 46 to 48, wherein a daily dosage of 0.5 mg of fingolimod is administered, based on fingolimod in form of the free base.

50. The method according to claim 45, wherein the S1P receptor modulator is siponimod or a pharmaceutically acceptable salt or co-crystal thereof.

51. The method according to claim 50, wherein siponimod is administered in form of siponimod hemifumarate or in form of a co-crystal of siponimod and fumaric acid.

52. The method according to claim 50 or 51, wherein the patient is administered siponimod if the state of MS in the patient is the second state, wherein the second state is SPMS state, or if the state of MS in the patient is the third state, wherein the third state is in-transition from RRMS to SPMS state.

53. The method according to any one of claims 50 to 52, wherein a daily dosage of 2.0 mg of siponimod is administered, based on siponimod in form of the free base.
